SEQUENCE LISTING (79 sequences)

SEQ ID NO: 1 - protein, 449 aa, unidentified
MAQFPSDFIWGVACASYQCEGGWDADGKGPNIWDDFCHRAGGSTVKNNDNGDVACDSYHRYPEDIALMKQHNIRAYRFSISWARVMPDGDGALNEAGLAYYDDLVNRLLENGIEPMVTLFHWDLPSALQYRGGWLNREMVDIFARYAGVIATRFKGRVKKYMTINEPQCIALGYYTDTMAPGWRCPDEDVARVFHIIALAHSAAQRAIKAVDPEALVGLVPCGRLCYPREETPENIESAYRASFDLTQRWAFNFNIIMDSVVLRRYDDSAPEAVRRFAATIPQSDWEAMETPDFIGVNVYNGTMVDAAGNDVDCYPGFPRTACKWPITPEVMHYGPMYLYRRYGLPMIISEDGLSCNDIIFRDGQVHDPKRIDFLHRYLTELSRAIAGGVPVKGYMQWSFLDNFEWASGYDERFGLIYVDYPTLRRIPKDSARWYANVIATNGACLEEG

SEQ ID NO: 2 - protein, 452 aa, unidentified
MKQFPEQFLWGVACASYQCEGAWNEDGKGPSIWDDFCHDPAGHIRNGDTGDIACDVYHRFREDIALMKKLGIKAYRFSISWPRVIPDGDGEVNEAGLRFYDELVDELLKSGIEPLITLYHWDLPSALQDKGGWLNRDIVAAFGRYAELIAERFRGRVRRYMTINEPPCITVLGYGSGIHAPGLRLNDEKLAQIFHILALAHSEAYRRIKAVSGPETRVGIVPCGRLCYPLEDTPENREAAYRATFDLSRERWGFTFNIILDSLIFRRYDDSAPEAVKRFAATVPACEWEQMEKPDFIGINVYNGECVDAEGKAAGRWPGFPLTATKWPVTPEVMHYAPLNLSRRYGLPMMITENGQSCNDRIFRDGQVHDPERIDFLHRYLLELHKAVEEGAPLEGYLQWSFLDNFEWSEGYGERFGIVYVDYPTQRRIPKDSAFWFGRIIESNGALLFSED

SEQ ID NO: 3 - protein, 449 aa, unidentified
MVKFPSDFIWGAACAAYQCEGAWNEDGKGPSIWDDFCHELGNQHVNNGDSGDVACDSYHRYREDVALMKQHGLKAYRFSISWPRVIPDGDGEVNEAGLAYYDALVDALLENGIEPMITLYHWDLPSALHLKGGWQNRQIAEWFARYARIIAERFKGRVTRYMTINEAQCITLLGYGIGVHAPGLKLPGEELARIYHNIALAHSAAQRAIKAVSPEAQVGFVPCGNLCYPVVDTPENRDAAYRASFAYTERWGFNFNIVLDSLVLRRYDDSAPAVLKKFAATIPASDWAQMEAPDFIGINVYQGQPVDGEGKPVPRPAGHPLTACKWPITPPVMHYGPLNVYRRYQLPIIISENGLSCNDVEFLDGKVHDPDRENYLHRYISELSRAIQDGTPVFGYLHWSFLDNFEWNSGYDERFGLIYVDYATQKRIPKDSAAWYAKVIETNGACLNG

SEQ ID NO: 4 - protein, 460 aa, Gemmiger formicilis
MNATDCITHFPKDFIWGAACASYQCEGAWNEDGKGPSIWDEFCHDTIDGKNLNISNGDIASDFYHHWREDIALMKAHNIRAYRFSVSWSRVLPDGEGKVNEQGLQWYSDVVDELLANGIEPMITLYHWDLPAALQDKGGWLNRDIIDVFAEYAAIIAEKLKGRVKRYMTLNEPACIVQAGYSKMLHAPGWRVSDEKMARIFHILALSHSAAKRAIKMIDPAAQVGIVTCGRLFWPERDTPENREAAYRASFDLSDAYWPFKHNILLDSLIFCRYDASIPAPVRRFAATIPESDWERMETPDFIGINVYEGPCINAARETVAPMYGSPVSACRWPITPEVLHYGPEYIYRRYRLPVLISENGISCNDMIFDDGRVHDPQRIQYLRRYLLALDKAIEEGTPVMGYLQWSVMDNMEWNSGYNERFGMFFVDYQTKQRIPKDSAAWYAKVIATNGQSLGEMPRF

SEQ ID NO: 5 - protein, 479 aa, Thermofilum pendens
MALKFGKEFKFGFSTVGVQHELGLPGSEFESDWIAWLRDPENIASGLVSGDDPFSGPGYWHLYREDHAIAEYLGMNAAWITVEWARIFPKPTTEVRAYVEQDGERITQVSLEESELERLLRLANREALSHYREIMSDWKSRGGFLIVNLFHWSLPLWLHDPVAVRSRGPDRAPSGWLDKRTVVEFAKFAALVARELDDLADAWYTMNEPMVVARLGYVSVSSGFPPGYLSLKAYEEAKVRLAEAHARAYDALREVSGKPVGLVESVSPVTVLGGESSLAELVLREQLAVLDAARFGTVGGEVREDLGGRLDWVGVNYYTRVVVSPGGPLGFRVESGYGYSCAPRGVSRDGRPCSDVGWEVYPEGLFEAISLVSKRYGLPVYITENGVADSRDALRPSFIVSHLYQVARLLEQGVDVRGYFHWNLTDNLEWAKGFSPRFGLVEVDYQTKKRRLRPSALVFREIALSREVPYEVALAGEWS

SEQ ID NO: 6 - protein, 427 aa, unidentified
MSFTKGFLIGASTAAHQVEGNNIHSDYWAQEHMPHSSFTEPSGIACDHYNRFEEDIRLMAKAGLNAYRFSIEWARIEPEEGQFDESELEHYRKVVRCCRKNGIEPLITLMHFTSPVWLIRQGGWEAESTVEYFRRYADFIVKNLGSEIKYICTINEANMGLQLAAIAKRFQLMAQQAQKSAKNAEGTVQVGMNFQKMMENMKYAAQENAEIFGTPQPQIFVSSRTEQGDTLVFRAHQAAKEAIKAINPDIQVGITLSLHDLQALPGGEAFAEKAWDEEFRHYLPFIQDDDFLGVQNYTRTQYGPKGQMPSPENAELTQMDYEFYPEALEHVIRSVHRDFKGNLIVTENGVATSDDTRRIEFIRRALQGVEHCLNDGIPVKGYCHWSLMDNFEWQKGYAMTFGMIAVDRTTLKRTPKESLQFLGSMIS

SEQ ID NO: 7 - protein, 480 aa, unidentified
MVKQFPPGFLWGGATAANQCEGAYDADGRGLSSVDVVPYGPERMKVSRGERKMLRCEEGFSYPSHEAIDLYHHYKEDIVLFAEMGFKCYRMSVAWTRILPNGDDDIPNEAGLKFYEDVFRECRRYGIEPLVTIDHFDTPIALIEKYGGWRDRRMIDAYIKYCTALFTRYKDLVKYWITFNEINMLLHMSFMGAGIYFEPGEDKEQVKYTAANNELLASARAVKLAHELMPGSMVGCMLAAGQFYPYSCNPADIWDGLEKDRDNYFFIDVQARGYYPVWAKKRMERAGIRLELSPEDEAVLREGTVDYVAFSYYCSRCTTADPEIFEAHKRPGNAVFASVENPHLPFTEWGWQIDPTGLRVTINTLYDRYQKPLFVVENGMGANDTLEPDGTVHDPYRIEYLRRHIEAMRDAVTEDGIPLLGYTAWGCIDLVSASSGEMKKRYGMIYVNKDDRGGGDLSRHRKDSFYWYKKVIASNGADLD

SEQ ID NO: 8 - protein, 476 aa, unidentified
MFKEDFLWGGATAANQFEGAWDVDGKGPSIPDHCTNGTRERSKLFTQTINPEYLYPSHKASDFYHHYKEDIALLAEMGYKCFRMSINWSRIFPTGMEKTPNEKGLEFYDKVFDECRKYGIEPLVTLSHYEMPLALGVEKDGWLSRETIDCFMRYVETVFARYRDKVRYWITFNEINAGQMPIGDIISTGMVKGYEGPINGIRRTEQERYQALHHQFVASARTVRLAHKKYPQFKVGNMLTFIAAYPVNCDPDNILLAAKYMQNMNWYCSDVQVKGAYPYYATAMWRDRDVILNITAKDIEDLENGTVDFMTFSYYMSICVGKEGEKDKVSGNLTGGFKNPYLESSDWGWQIDPVGIRYALNAAYDRYRIPLMIVENGLGAFDKVEEDGSVHDDYRIDYMRRHIRQMKLATEDGVELMGYTNWGCIDLVSLTTGEMRKRYGQVFVDKYDDGTGTLKRSRKDSFFWYRNVIRTNGMEI

SEQ ID NO: 9 - protein, 449 aa, unidentified
MAKYDFPKDFNWGTATASYQVEGGAHEDGKGPSIWTEFEKRPGAIFNGDNGDVASDQYHHWKEDIELMKYLGLRSYRFSMAWSRVIPEGRGAINVAGLDYYKRLCDALLENGIEPYMTFYHWDLPLALQKEFGGWESRETVKYFGEYVERISKELKGRVKNYFTTNEFLACSDVGYGMGSIAPGLKLPAKRLNQVRHHVLLAHGTALAALRATSPEAKVGLAENPWFMVPLIDTPEHVEATKLAFREENAHFLTAIMEGKYLDCYLEKCGADAPEFTDDDMKIIGGKVDLLGLNIYFGKYVCKEDDKPYRIFRDDIQSTKAGRPGLYYEPDAIYWGARIVTELWNVPELIVSENGTAMPEDNIDVDSGRVYDLGRIKYLRNYLTSMARAISEGYPIKGYFHWSLVDNLEWNQGLQPRFGLTYIDFHTLKRTLKMSGEWYRELIRTGRIV

SEQ ID NO: 10 - protein, 448 aa, unidentified
MVQFPADFTWGVACASYQCEGGWNADGKGPSIWDDFCHELNGHHVKNDDSGDVACDSYHRYREDVALMKAHNIRAYRFSISWPRVIPDGDGAVNEAGLAYYDALVDLLLENGIEPMVTLYHWDLPSALQHRGGWQNRQIADWFARYADIIARRFAGRVKRYMTINEAQCITELGYGRGVLAPGLQLPDEELARIYHNIALAHSAAQRAIKAVSPDAVVGFVSCGKFCFPEHDTPEAVDAAYRAMFEMDEGWGFNFNVVLDSLILRRWDDSAPAAVRRFVETIPPEDWDLMEAPDFVGINVYNGGMVDDAGKPVPHVPGHPITACKWPITPRVMRYGPLLIHRRYGLPMIITENGLSCNDIRFMDGQVHDLKRIDFLHRYLTELSKAIADGAPVLGYLQWSFLDNFEWASGYDERFGIIYVDYQTLERTPKDSARWYAKVIETNGACLN

SEQ ID NO: 11 - protein, 471 aa, unidentified
MNFPKDFLWGVATSSYQIEGAEHEDGRCKSVWDDFYKIPRKVVDEKSGAIACDHYHRYKEDVQLIKNLGVKAYRFSVAWPRIFSYDSDSRNGVVKGNLNQKGLDFYDRLIDELLQNGIEPWLTLFHWDLPYELEKKGGWRNRDIHHWISDYSAEIARRYSDRVTHFFTLNEMPCILGGYRGWFAPGLEVNEREVFNIIHHMLLSHGSMVQAVRANAKQNVLLGCAHNGLGHYPASESKEDYEAFIKAMNCIEAAPGRYAPQEGSGILSGDSLTYYLDPIHFGKYPDKAFELFADKMPEIKDGDMKLISSPVDYQGINIYEGRPITAGSAPGKKDGGWHIEPFEEGYNITAAKWPITPKSMNHYFKFISDRYKKPVYVSENGMSNADIVSLDGKCHDPQRIDFTERYLAELKKAIDSGADVKGYFHWSLMDNYEWRNGYTERFGLVHVDYQTQKRTPKDSYWWYKELVEKYK

SEQ ID NO: 12 - protein, 440 aa, unidentified
MSFRKDFAWGAATAAFQIEGAWNEDGKSPSIWDVFCTQPGKIEDKSDGTVACDHYHRYKEDVKLMSELGLKAYRFSIAWPRVIPDGRGKVNEKALDFYSNLVDELLKYNITPYVTLYHWDLPYCLYLKGGWMNPEISDMFEEYTRAVAKRLGDRVKHYITFNEPSVFLGCGCLEGSHAPGHKMGTRDLLNMGHNVLLSHGKAVRALRELVPDAEVGITLATMPAIPVAKKNEEEAYESYFYCDKNTFVWSDAFWVDPIVLGKYPEKLLSECKDIFPAFTDEDMKLISQKIDFLGQNIYQGRYVGEWKRPAGTAHTELSWDVFDDALEWGIKHFTKRYRLPMYITENGLSCHDWVSLDGKVHDPNRIDFLHRYLRGLKKAAESGCDVRGYFQWSLMDNFEWAKGYNPRFGMIFCDYTTQKRIPKDSAYWYKEVIETNGENL

SEQ ID NO: 13 - protein, 450 aa, unidentified
MFTRPDLPKDFLIGAATASYQVEGAANEDGRTSCIWDDFAKVPGKVFQCQDGSVAADQYHRYKEDIELMAKLGFKAYRFSVSWSRVLPNGGKKVNPKGIEYYRNLCIELHKHNMKACCTIYHWDMPSEIQAKGGWSNRQTSYELAYLAKVLFEELGDLVDMWITINEAMCITVLGYLLGIHAPGIKDKNQFIRSVHHVNLAHGLVLQEYRKSGLKAPIGITHNLETPRPASKDEKDRLAVQHHIALRDGIFMDPIFKKAYPTYMTDELGWVFPIEDGDFELISQPMDFLGINYYSEHVITWSDTEPFNVKEVPRWEEKMTGIGWCITPHGLLRLLKWVTEYTNSTIPIYITENGCCSADKLETDPVTKQERVHDTQRVRYLSDHLNICAEAIKNNIPLKGYFCWSFIDNYEWTYGYSMRFGLVYCDYQTQRRIPKDSAYFMRDVMAGYGD

SEQ ID NO: 14 - protein, 446 aa, unidentified
MAYFPKDFLWGVACASYQCEGGWDADGKGRNIWDDFCREPGKVKYGDTGDTACDTYHRIDEDVALMKKFGVQAYRFSLSWARILPEGDGEVNEAGLEYYSRVVDLLLENGIEPMVTLYHWDLPSALQYKGGWLNRDIVKAFGRYADIVSKRFGDRVTRYMTINEPQCITALGYGKGVLAPGWVLPDVDLARIYHNIALSHSEAQRRIRGNVPGAQVGIVPCGQLCYPKEETEENIEAAYRASFDLSHGWWAFKFNICLDNLIRRGWDDTAPETLRRFQDTVPASDWQLMETPDFLGMNVYNGDCVDGSGRNVPQPSGHPVTGCKWPVTPEVLHYGPIHLYRRYQLPLYITENGLSCNDVVSLDGLVHDPARIDFLHRYLRELSKALQAGIPLRGYLHWSFLDNFEWASGYDERFGLIHVDYQTLVRTPKDSAAWYRRVIETNGAEL

SEQ ID NO: 15 - protein, 488 aa, Thermococcus celer
MYKFPRDFVFGYSWSGFQFEMGLKGSEVPNSDWWVWVHDMENIMTGLVSGDLPENGPAYWHLYSKDHDMAEKLGMDAIRGGIEWARIFPEPTFDVRVTVERDEEGRITSVDVPESAIEELEKRANLEALEHYKRIYSDWRERGKVFILNLYHWPLPLWLHDPIKVRRFGPDRAPSGWLDDRSVVEFAKFAAFVAYHLNDFVDSWSTMNEPNVVYENGYGRPNSGFPPGYLSFEAVEKAKLNLIYAHARAYDAIKEFSEKPVGVIYAYTWLDPLSEEIAEDVRKIRENELYSFVDSVHFGESRTVGEGREELKGRVDWLGVNYYSRIAFDRVNGHVVPLPGYGFSGVRKGYAKSGRPCSDFGWEIYPEGLEKLLRELNERYGLPMMITENGMADEADRYRSYYLVSHLRAIHSAIEAGADIRGYLHWSLTDNYEWAKGFQMKFGLLKVDWESKRRYIRPSALVFKEIATQKAIPEELSHLSDLRPLLQD

SEQ ID NO: 16 - protein, 484 aa, Vulcanisaeta sp.
MSLKFPKDFGFGFSTAGFQHEMGLPGSEYESDWWVWVHDPENIAAGIVSGDLPENGPGYWHLYKSDHDIAFSLGMDTLRLGIEWARVFPKPTFEVNVNADIRDGSVVSVDVSEEALRRLDGLANRDAVQHYIEIIKDWKDRGGKLIVNLYHWPLPLWVHDPLVVRRSGPNNAPTGWLDPRTVVEFAKYAAYLAWRLGEFVDMWSTMNEPNVVFSNGYLYVKSGFPPGYLGIELMLRARGNLMTAHARAYDALREFSKAPIGIIYAISDVQPLTKDDEEAAKAYEEAGQVSFLDAITKGSGREDLRGRLDWLGINYYSRTVVTTAKSQSSILPPARVVPGYGFACGPNAVSRDGRPCSDFGWELYPEGLYNVLTRYWGRYGLPIIVTENGIADARDQWRSWFIVSHLYQLHRALGQGVDVRGYLHWNLIDNYEWASGFRMKFGLVQVDYNTKKRYLRPSALVFREIARNKEIPEYLTHMIQSPTI

SEQ ID NO: 17 - protein, 486 aa, Thermococcus guaymasensis
MWKFPKDFLFGYSWSGFQFEMGLEGSEVPNSDWWVWVHDTENIFSGLVSGHLPENGPAYWHLYKQDHDIAEGLGMEAIRGGIEWARLFPKPTFDVKVDIEKDEDGNIVAVDVPERAIEEMEKLADMKALEHYREIYSDWKGRGKVFILNLYHWPLPLWLHDPIAVRRLGPDRAPSGWLDERSVVEFVKFAAFVAYHLNDLVDMWSTMNEPNVVYEQGYTRPNSGFPPGYLSFESSTKAARNMAQAHARAYDVIKEHSKAPVGLIYSFVWHDALNEEAEDIVKEIRKRHYEFVTAVHSGSSGLLGERPDMKGKLDWIGVNYYTRVAYRMNNGSIEVPPGYGYMCERGGFAKSGRPASDFGWEIYPEGLENILRDLHRIYGLPMMITENGIADAADRYRPYYLVSHLKAVHSAMEAGADVRGYLHWSLTDNYEWAQGFRMRFGLVHVDFETKKRYLRPSALAFREIATRKEIPEELSHLADLTPLMRD

SEQ ID NO: 18 - protein, 456 aa, unidentified
MSLKFPKDFKFGFSEAGFQFEMGLPGSENPHSDWWTWVHDQENITAGIVSGDLPENGPGYWHLYQKDHEIADSLGMDSARLGIEWSRLFPKPTFNIKADVEKDSAGNIISVEVGEKSLEELDKIANKEAVEHYRRIFEDWRKRGKLLIINLYHWPMPVWLHDPIKVRKLGPDRAPAGWVDERSVVEFTKFAAYVAWKLGDLPDMWSTMNEPNVVYTQGYVSIKSGFPPGYLSVEASLKAAKHLIEAHARAYDVLKKMTKKPVGIIYATAEIEPLTTEDKEIAEAAYAQHNFSFMDAIFTGTSQLVGGERKDLARHLDWIGINYYSRLVVTRAKTAAGWRVVEGYGFACQPRGISRAGRPCSDFGWEVYPEGLYSVVKRFWERYRLPMLITENGIADSVDALRPRYLVSHLAQVHKLVSEGVELKGYLHWALTDNYEWAQGFRMRFGLVYVDYETKK

SEQ ID NO: 19 - protein, 486 aa, Ignisphaera aggregans
MGLKYPKEFIFGFSESGFQFEMGLPGSEDPNTDWWVWVHDPENIASTLVSGDFPENGPGYWHLYRQDHDIAERLGMDGARIGIEWSRIFSKPTFDVKVDVARDERGNIVYIDVAEKALEELDRIANKDAVNHYREILSDWKNRGKKLIINLYHWTLPLWLHDPIKVRKLGIDRAPAGWVDERTVIEFVKYVAYIAWKLGDLPDLWCTMNEPNVVYSIGYINIKIGYPPGYLSFEAASKAMKHLVEAHARAYEVLKRFTNKPVGIIYVTTYHEPLKESDRDVAEAAMYQAVFDFLDSITIGRSMSIGERKDLEKHLDWLGINYYSRLVVERYGNAWRVLPGYGFACIPGGTSLAGRPCNDAGWETYPEGLYIMLKRCWERYRLPIIVTENGTADAIDRLRPRYLATHLYQVWKALSEGVDIRGYLHWALVDNYEWSSGFRMRFGLVHVDFETKKRYLRPSALLFREIASSKEIPDEFMHMTQPQILI

SEQ ID NO: 20 - protein, 485 aa, unidentified
MKIPKEFMLGASLSSFQFEGGFRGDEDPNNDWWIWVHDWENIIAGIVSGDFPENGPGYWRLFRQDHDLAEKLGMNTLRVGIEWSRIFPRPTFDVKVTVDKDEDGNILHVDIDEKALAKLDEIADQDAVKHYIEMYSDWKNRGKQLIINLYHWPLPLWIHDPIKVRKYGPDRAPSGWLDEKTIIEFVKYAAYVSWKLRDLADMWSTMNEPNVVYEQGYMFIKNGFPPGYLSFEAAEKAKKNLIYAHARAYEVVKKITGKPVGIIYALPYIESLNGEKETLEAIKSYRIYEFLDLIIKGKSVRNPILRKELASRADWLGVNYYSRIVFKFIHGKPIVLQGYGFFCSSSGVSKMGLPCSDFGWEIYPQGLYLLLKEIHTRYNGLPIIVTENGISDKADKLRPKYLVSHLYNTLKARNEGVPVKGYLHWSLIDNYEWAQGFRQRFGLVIVDFNTKKRYIRPSALVFREIALSQEIPEELMHLTHVEPLI

SEQ ID NO: 21 - protein, 486 aa, Caldivirga maquilingensis
MIKFPSDFRFGFSTVGTQHEMGTPGSEFVSDWYVWLHDPENIASGLVSGDLPEHGPGYWDLYKQDHSIARDLGLDAAWITIEWARVFPKPTFDVKVKVDEDDGGNVVDVEVNESALEELRRLADLNAVNHYRGILSDWKERGGLLVINLYHWAMPTWLHDPIAVRKNGPDRAPSGWLDKRSVIEFTKFAAFIAHELGDLADMWYTMNEPGVVITEGYLYVKSGFPPGYLDLNSLATAGKHLIEAHARAYDAIKAYSRKPVGLVYSFADYQPLRQGDEEAVKEAKGLDYSFFDAPIKGELMGVTRDDLKGRLDWIGVNYYTRAVLRRRQDAGRASVAVVDGFGYSCEPGGVSNDRRPCSDFGWEIYPEGVYNVLMDLWRRYRMPMYITENGIADEHDKWRSWFIVSHLYQIHRAMEEGVDVRGYFHWNLIDNLEWAAGYRMRFGLVYVDYATKRRYFRPSALVMREVAKQKAIPDYLEHYIKPPRIE

SEQ ID NO: 22 - protein, 493 aa, Thermoproteus uzoniensis
MRKFPSGFRWGWSGAGFQFEMGLPGSEDPNTDWFAWVHDPENIAAGLVSGDFPENGVAYWHLYKQFHDDTVKMGLNTIRFNTEWSRIFPKPTFDVRVHYEVREGRVVSVDITEKALEELDKLANKDAVAHYREIFSDIKSRGLYFILNLYHWPMPLWVHDPIKVRRGDLSGRNVGWVAETTVVEFAKYAAYVAWKFGDLADEFSTFNEPNVTYNLGFIAVKAGFPPGYLSFQMARRAAVNLITAHARAYDAIRLTSKKPVGVIYAASPVYPLTEADKAAAERAAYDGLWFFLDAVAKGVLDGVAQDDLKGRLDWLGINYYSRSVVVKRGDGYAGVPGYGFACEPNSVSRDGRPTSDFGWEIYPEGLYDILTWAWRRYGLPLYVTENGIADQHDRWRPYYLVSHLAQLHRAIQDGVNVKGYLHWSLTDNYEWASGFSKKFGLIYVDLSTKRHYWRPSAYIYREIASSNGIPDELEHLEKVPVASPEVLRGLRSL

SEQ ID NO: 23 - protein, 483 aa, unidentified
MISLPGIRFGWSQAGFQSEMGLPGSEDPNSDWFAWVHDKENIAAGVVSGDLPEYGPAYWHRFREFHDAAERMELKIARIGVEWSRVFPKPTLDVQVDIEQRGDMVTHVDVSQSQLEKMDAIASKDAVEHYRTIFSDLKRRGIEFVLNLYHWPLPLWIHDPVAVRRGEKTERTGWLSTRTVVEFAKFAAYISWKLDDLVDAYSTMNEPNVVWGAGYTSVKSGFPPGYLSFAHSSRAMYNMVQAHARAFDVLKTHKKPVGIIYANSDFQGLTAGDADVASKAEFDNRWRFFEAIVNGDLGGYRDDLKGRLEWIGVNYYTRSVVRKAGEGYVVVRGYGHACERNSLSADGRPTSDFGWEFYPEGLGNVLVKYREKYGLPLYVTENGIADEADYQRPYYLVSHIYQVYQALRRGADVKGYLHWSLADNYEWASGFTPRFGLLRVDYTNKSLFWNPSAFVYKEIAGSNGIPDQLEHLNRVPPTRGLRR

SEQ ID NO: 24 - protein, 465 aa, Fervidobacterium changbaicum
MFPNSFMFGASLSGFQFEMGNPSDPSELDTQTDWFVWVRDLENLLNGIVSGDLPESGAGYWKSYEKIHQLAVDFGMDTLRIGIEWSRIFPSSTREIPFGEGMLEKLDSIANKDAVEHYRKIMEDMKSKGLKVFVNLNHFTLPLWLHDPLAVRKGKPTDKLGWVSDDAPVEFAKYAEYIAWKFGDIVDYWSSMNEPHVVAQLGYFQILAGFPPSYFNPEWYIKSLRNEATAHNLTYDAIKRHTDKPVGVIYSFTWYDTLKPNNSEIFENAMWLANWNFMDQVKDKVDYIGVNYYTRAMIDKLPKPIEIQDFELNWYVVRGYGYACQEGGFALSGRPASEFGWEIYPEGLYYLLKAIYERYNKPLIVTENGIADQNDKYRAQVLISHLYAVEKAMNEGVDVRGYLHWSIVDNYEWAKGYSKRFGLAYTDFEKKLYIPRPSMYVFREIAKTRSIDQFKGYDPYGLMKF

SEQ ID NO: 25 - protein, 465 aa, Fervidobacterium sp.
MFPKDFMFGVSMSGFQFEMGWGDERDLDPNTDWFVWVREPGNLVNGVVSGDLPEFGAGYWLNYEKIHQLAVDFGMDTIRIGIEWSRIFPTSTESVDVRDPNFLDKLDELANKKAVEHYRKIMEDIKSKGLKLFVNLNHFTLPLWLHDPVAVHYGRPTDKLGWVSERTVHEFAKYVAYMAKYGDIVDLWSTMNEPHVVSQLGYFSVSAGFPPAYFNPEWYILATKHLAMAHNLGYDMIKRFSDKPTGVIYSFTWYDTLNPNDREILEEAMYLTNWFFMDMVKEKLDYVGVNYYTRTVIDRVEQPLAMGNFNVRWRILKGYGYACDEGGVALSGRPASDFGWEMYPEGLYYVLKAVSERYSKPIIVTENGVADWNDRLRSTHLISHLYYVERALEDGIDVKGYLHWSIVDNYEWAKGYSKRFGLAWTNFQTKTYHPRPSMYIFRDIIRARTTKEFIGFDPYKVRTEL

SEQ ID NO: 26 - protein, 466 aa, Fervidobacterium gondwanense
MFPKDFMFGASLSGFQFEMGNPNDPKEVDPNTDWFVWVREPENLVNGIVSGDLPEYGAGYWKNYEKVHQLAVDFGMDTLRIGIEWSRVFPTSTREVPTGDGMLEALDKIANKEAVEHYRKIMEDMKSKGLKVFVNLNHFTLPLWIHDPISVHKGIPTDKLGWVSDDTPIEFAKYAEYIAWKFSDIVDYWSSMNEPHVVAQLGYFQILAGFPPSYFRPEWYIKSLVNEAKAHNLAYDAIKKYTSRPVGIIYSFIWYDTVNPQDRDIFENAMWLTNWYYIDMVKDKADYIGINYYTRSLIDRLPASGMKFGDFELNWYPLRGYGYACPEGGMSLSGRPASEFGWEVYPEGLYNLIKAIYERYKKIIIVTENGIADEKDKYRSHYLISHLYAVEKAMNEGANVIGYLHWSIVDNYEWAKGYSKRFGLAYTDLEKKIYVPRPSMYIFREIAKTKSIEQFKDYDPYKLMKF

SEQ ID NO: 27 - protein, 491 aa, Sulfolobus acidocaldarius
MLSFPKGFKFGWSQSGFQSEMGTPGSEDPNSDWHVWVHDRENIVSQVVSGDLPENGPGYWGNYKRFHDEAEKIGLNAVRINVEWSRIFPRPLPKPEMQTGTDKENSPVISVDLNESKLREMDNYANHEALSHYRQILEDLRNRGFHIVLNMYHWTLPIWLHDPIRVRRGDFTGPTGWLNSRTVYEFARFSAYVAWKLDDLASEYATMNEPNVVWGAGYAFPRAGFPPNYLSFRLSEIAKWNIIQAHARAYDAIKSVSKKSVGIIYANTSYYPLRPQDNEAVEIAERLNRWSFFDSIIKGEITSEGQNVREDLRNRLDWIGVNYYTRTVVTKAESGYLTLPGYGDRCERNSLSLANLPTSDFGWEFFPEGLYDVLLKYWNRYGLPLYVMENGIADDADYQRPYYLVSHIYQVHRALNEGVDVRGYLHWSLADNYEWSSGFSMRFGLLKVDYLTKRLYWRPSALVYREITRSNGIPEELEHLNRVPPIKPLRH

SEQ ID NO: 28 - protein, 489 aa, Vulcanisaeta distributa
MDISFPKSFRFGWSQAGFQSEMGTPGSEDPNTDWYVWVHDPENIASGLVSGDLPEHGPGYWGLYRMFHDNAVKMGLDIARINVEWSRIFPKPMPDPPQGNVEVKGNDVLAVHVDENDLKRLDEAANQEAVRHYREIFSDLKARGIHFILNFYHWPLPLWVHDPIRVRKGDLSGPTGWLDVKTVINFARFAAYTAWKFDDLADEYSTMNEPNVVHSNGYMWVKSGFPPSYLNFELSRRVMVNLIQAHARAYDAVKAISKKPIGIIYANSSFTPLTDKDAKAVELAEYDSRWIFFDAIIKGELMGVTRDDLKGRLDWIGVNYYSRTVVKLIGEKSYVSIPGYGYGCERNSISPDGRPCSDFGWEFYPEGLYDVIMKYWSRYHLPIYVTENGIADAADYQRPYYLVSHIYQVYRAIQEGANVKGYLHWSLTDNYEWASGFSMRFGLLQVDYSTKKQYWRPSAYVYREIAKSKAIPEELMHLNTIPPTRSLRR

SEQ ID NO: 29 - protein, 481 aa, synthetic construct
MVENNFPEDFKFGWSQSGFQSEMGYDNAMDDKSDWYVWVHDKENIQSGLVSGDMPENGPGYWNNYKSFHEAAQNMGLKMARIGVEWSRLFPEPFPEKIMADAKNNSLEINNNILSELDKYVNKDALNHYIEIFNDIKNRNIDLIINMYHWPLPVWLSDPVSVRKGIKTERSGWLNDRIVQLFALFSSYIVYKMEDLAVAFSTMNEPNVVYGNGFINIKSGFPPSYLSSEFASKVKNNILKAHSLAYDSMKKITDKPVGIIYANTYFTPLDPEKDNDAIAKADSDAKWSFFDPLIKGDKSLGINGNKLDWIGINYYTRTMLRKDGDGYISLKGYGHSGSPNTVTNDKRPTSDIGWEFYPEGLEYVIMNYWNRYKLPMYVTENGIADNGDYQRPYYLVSHIASVLRAINKGANVKGYLHWSLVDNYEWALGFSPKFGLIGYDENKKLYWRPSALVYKEIATKNCISPELKHLDSIPPINGLRK

SEQ ID NO: 30 - protein, 472 aa, Pyrococcus furiosus
MKFPKNFMFGYSWSGFQFEMGLPGSEVESDWWVWVHDKENIASGLVSGDLPENGPAYWHLYKQDHDIAEKLGMDCIRGGIEWARIFPKPTFDVKVDVEKDEEGNIISVDVPESTIKELEKIANMEALEHYRKIYSDWKERGKTFILNLYHWPLPLWIHDPIAVRKLGPDRAPAGWLDEKTVVEFVKFAAFVAYHLDDLVDMWSTMNEPNVVYNQGYINLRSGFPPGYLSFEAAEKAKFNLIQAHIGAYDAIKEYSEKSVGVIYAFAWHDPLAEEYKDEVEEIRKKDYEFVTILHSKGKLDWIGVNYYSRLVYGAKDGHLVPLPGYGFMSERGGFAKSGRPASDFGWEMYPEGLENLLKYLNNAYELPMIITENGMADAADRYRPHYLVSHLKAVYNAMKEGADVRGYLHWSLTDNYEWAQGFRMRFGLVYVDFETKKRYLRPSALVFREIATQKEIPEELAHLADLKFVTRK

SEQ ID NO: 31 - protein, 487 aa, Terribacillus goriensis
MYKFPRDFLFGYSWSGFQFEMGLPGSEVPNSDWWAWVHDIENIAAGLVSGDLPENGPAYWDLYKKDHDIAESLGMDAIRGGIEWARIFPKPTFDVKARVERDEKGNIVSVEVPESSIKELEKIADMNALEHYREIYADWKERGKTFILNLYHWPLPLWLHDPLKVRKLGPDRAPAGWLDDKSVVEFAKFAAFVAYHLDDLVEVWSTMNEPNVVYQNGYTRPTHGFPPGYLSFEAERKAKMNLIQAHARAYDVIKEYSDKDVGVIYAYTWPDPLREDIEEEVRAIRERELYSFVDAVHFGKAADVEERDDLKGRVDWLGVNYYSRIAFDMVNGHVLPVPGYGFSGERGGYARSGRPCSDFGWEIYPEGLEQLLKDLAKRYGLPMMITENGIADAADRYRPHYLVSHLKAVHEAMKEGADVRGYLHWSLTDNYEWAQGFRMRFGLVYVDMETKKRYLRPSALVFREIATRKEIPEELEHLSSLDFLVRR

SEQ ID NO: 32 - protein, 467 aa, Fervidobacterium nodosum
MMFPKDFLFGVSMSGFQFEMGNPQDAEEVDLNTDWYVWVRDIGNIVNGVVSGDLPENGSWYWKQYGKVHQLAADFGMDVIRIGTEWSRIFPVSTQSVEYGSPDMLEKLDKLANQKAVSHYRKIMEDIKAKGLKLFVNLYHFTLPIWLHDPIAVHKGEKTDKIGWISDATPIEFAKYAEYMAWKFADIVDMWASMNEPHVVSQLGYFAINAGFPPSYFNPSWYIKSLENEAKAHNLSYDAIKKYTNNPVGVIYSFTWYDTVNKDDKESFENAMDLTNWRFIDMVKDKTDYIGVNYYTRAVIDRLPTTIDFGEFKMNWYTLRGYGYSCEEGGFSLSGRPASEFGWEIYPEGLYNILIHVYNRYKKDIYVTENGIADSKDKYRSLFIISHLYAIEKALNEGIPIKGYLHWSIIDNFEWAKGYSKRFGLAYTDLSTKKYIPRPSMYIFREIIKDKSIDKFKGYDPYNLMKF

SEQ ID NO: 33 - protein, 465 aa, Thermosipho africanus
MFSKDFLFGASLSGFQFEMGNPNNEEELDKNTDWFVWVRDLGNIINGKVSGDLPEYGAGYYTNYKAVHNLAKEFGMNALRIGIEWSRIFKESTKDISLDDPNMLEKLDQLADKKAIEHYRDVLEDIKSKGLVAIVNLSHFTLPLWLHDPINVHKGKETEKLGWVSDDAPIEFAKYAEYIAWKFKDIVDMWSSMNEPHVVSQLGYFQTSAGFPPSYFNPSWYLKSLENQALAHNLAYDAIKKHTGKPVGVIYSFTWYDTVNNDEEIFESAMFLNNWNYMDRVKDKIDFVGVNYYTRAVIDRLLVPIKIDNYELNWYTLSGYGYSCVEDGFANSKRPSSEIGWEIYPEGLYNILKEIYNRYGKQIYITENGIADSSDKYRSFYIISHLYAVEKAINEGVPVKGYLHWSIIDNYEWAKGYGKRFGLAYTDFERKTYIPRPSMYILREIIKERSIDKFKGYDPYGLMNF

SEQ ID NO: 34 - protein, 476 aa, unidentified
MTIQFDADFVWGAATSGPQAEGTFHKKHENIFDYHYHTRPQDFYHNVGPDVASNFYNDYENDLALLKQAGVQALRISIQWTRLIDDLEAGTVDPVGADYYRRVFKTMHQLGITPYVNLHHFDLPVTLQHQYGGWQSKHVVDLYVKFATRCFELYSDQVTHWFTFNEPKVIVDGQYLYQFHYPNIVDGRLAVQAAYNLNLASAKAVAAFRQINRQSQGTIGTIVNLTPVYPASQAPEDLAAARFAEQWANDLYLEPAIHGRFPEELVARLKRDGVLWEATSDELAVIAANRIDVLGVNYYHPFRVQAPAVSPDSLQAWLPDIYFDNYDMPGRKMNLDKGWEIYPDALYDIAMTIKRRYDNLPWFVAENGIGVANEERFLKDGMVQDDYRIQFMTDHLRFLSQAITEGANCHGYFVWTGIDCWSWLNAYKNRYGLIRNDLCNQTKSLKKSGHWFSQVAATGLVAPTLRPFESEEKNHG

SEQ ID NO: 35 - protein, 466 aa, Streptococcus equinus
MKQSKRRYQFPEGFLWGSSTSGPQSEGTVSGDGKGPSNWDYWFSLEPDKFHHQIGPEVTSTFYTNYKSDIALLKETGHTAFRTSIQWSRLIPEGVGQVNPKAVAFYREVFQEIMAQDIKLIVNLYHFDLPYALQGKRGWEAKETVWAYETYAKTCFELFGDLVDTWITFNEPIVPVECGYLGHYHYPCKVDAKAAVQVAYHTQLASSLAIKACHELYPKHRISIVLNVTPAYPRSDQPEDVKAARIAELFQTKSFLDPSVLGVYPEELVVLLEAADLLPQYSADELAIIKNNPVDFLGVNYYQPLRVQAPSKTRQDGEPITLASYFEPYDMPGKKVNPHRGWEIYEQGLYDIALNLKEHYGNIDWLVTENGMGVEGEEAFLVDGQIQDDYRIAFIEDHLIQLHRALEEGANCKGYLLWTFIDCWSWLNAYKNRYGLVALDLETQKRTLKKSGHWFKTLSQTNGFDK

SEQ ID NO: 36 - protein, 465 aa, unidentified
MQYQLPKDFFFGGAMSGPQTEGRWQDDGRIPSIWDTWSNLDITAFHNRVGSYGGNDFSSRMEEDFELLKSIGMDSVRTSIQWSRLLDIDGNLNPEGERYYHQLFATAKKVGIEIFVNLYHFDMPEYLFNRGGWESREVVEAYAHYARIAFETFGKEIRYWFTFNEPIVEPEMRYTVGGWFPFVKNYSRARAVQYNISLAHALGVREYRRAKAAGFMLEDSRIGLINCFAPPYTKDNPSEADLEALRMTDGVNIRWWLDLVTKGELPQDVIDTLQSRGVDLPIRPEDKLILADGVVDWLGCNYYHPERIQAPAKDTDENGIPNFADPYVWPEAEMNVSRGWEIYPQGLYDFAMKVRDEYPELEWFVSENGMGVEREDLKKDENGVIQDDYRVDFVRRHLEWIARAIQDGAKCRGYHYWAIIDNWSWANAFKNRYGFIEVDLEDNYNRRLKKSAKWLKQIATTHIVD

SEQ ID NO: 37 - protein, 411 aa, Chloroflexus aurantiacus
MQQFAFPTGFLWGAATSAHQVEGNNINSDSWVLEHLPDTIYAEPSGDACDYYHRYPEDIALLAQLGFNAYRFSIEWARIEPEEGEFSFASLEHYRRMLATCHEHGLKPVVTLHHFTSPRWLIRAGGWLDPKTPDRFVRYCERVVHYLGDLIAGACTFNEPNLPVLLSKIMPASPLASPFWRAAAAEFAVTPDRLGIFQFVSQPRMREIIFAAHRRAFEVLHDGPGSFPVGMTLALVDIHAGPDGERMAAEFRRELAEVYLEQLREDDFVGVQTYSRLVVGPAGIIPPGDDVEKTQTGEEYYPEAIGGTIRHAAAVAGIPVVVTENGLATTDDTRRVEYFRRALRSVAECLIDGIDVRGYFAWSALDNFEWISGYKPKLGIIAVDRTTQARTPKPSAYWLGNVARFNYCVFD

SEQ ID NO: 38 - protein, 391 aa, Bifidobacterium dentium
MRETYEFPQEFIWGASTAAHQIEGNNVASDWWAREHAECADLSEPSGDAADSYHRYGEDIRMLADAGLGMYRFSIEWARIEPAEGCFSKAQLLHYRHMIDACHENGIEPMVTLNHMTLPLWLAVKGGWLNDGAVDYFDRYVRYLMPILHDVTWVCTINEPNMVALTRGGTEGSDFVSASLPAPDLDISAALVEAHREARGILSENPRIKSGWTIACQAFHAMPGCEQEMEEYQYPREDYFTEAAAGDDFIGVQAYLRTFIGKDGPVPVPEDAERTLTGWEYFPPALGIAIRHTWNVAGHTPIIVTENGIATADDRRRIDYTFGAIAGMHDAMADGVDVRGYLHWSLLDNYEWGSFAPTFGLACWDKDTFERHPKPSLNWLGMIAKTGVMSR

SEQ ID NO: 39 - protein, 400 aa, Streptomyces rochei
MTRTSLPFPDGFLWGASTAAHQIEGNNVNSDWWRKEHDPAANIAEPSLDACDSYHRWEQDMDLLAELGFTDYRFSVEWARIEPVPGTFSHAETAHYRRMVDGALARGLRPMVTLHHFTVPQWFEDLGGWTADGAADLFARYVEHCAPIIGKDVRHVCTINEPNMIAVMAGLAKTGDQGFPPAGLPTPDEETTHAVIAAHHAAVKAVRAIDPDIQVGWTIANQVYQALPGAEDVTAAYRYPREDVFIEAARGDDWIGVQSYTRTKIGADGPIPAPEDAERTLTQWEYYPAAVGHALRHTADVAGPDMPLIVTENGIATADDARRVDYYTGALEAVSAALEDGVNIHGYLAWSALDNYEWGSYKPTFGLIAVDPVTFERTAKPSAVWLGEMGRTRQLPRAER

SEQ ID NO: 40 - protein, 469 aa, Clostridium acetobutylicum
MKFPKDFFLGAASASYQVEGAWNEDGKGVSNWDVFTKIPGKTFEGTNGDVAVDHYHRYKEDVKLMAEMGLDSYRFSVSWPRIIPDGDGEINQKGIEFYNNLIDECLKYGIVPFVTLYHWDMPEVLEKAGGWTNKKTVDAFVKYAKACFEAFGDRVKRWITFNETIVFCSNGYLSGAHPPGITGDVKKYFQATHNVFTAHARSVIEYKKLKQYGEIGITHVFSPAFSVDDKEENKAAAYHANQYEITWYYDPILKGKYPEYVIKNIEKQGFLPDWTDEELNTLREAAPLNDFIGLNYYQPQRVIKNHDTGEKIERTRENSTGAPGNASFDGFYRTVKMDDKTYTKWGWEISPESLILGLEKLKEQYGDIKIYITENGLGDQDPIIEDEILDMPRIKFIEAHLRAIKEAISRGINLKGYYAWSVIDLLSWLNGYKKQYGFIYVDHKHNLDRKKKLSFYWYKKVIEERGKNI

SEQ ID NO: 41 - protein, 468 aa, Sebaldella termitidis
MERLPEDFIFGAATAAFQAEGAVNEDGRGKCYWDEYLHRAESTFNGDTASDFYHKYREDTALCREYGINGIRISIAWTRIIPDGSGKVNQKGIDFYNDMINACLEAGVEPYVTLHHFDTPLELFKNGDWLNRENTEHFVRFAKICFENFGDRVKKWITINEPWSVVAGQYIIGHFPPNIKYDVPKAVQAMHNMCTAHAKAVIEYKKMNLNGEIGIIHILESKYPISEKPEDIRAALLEDTLANKFMLDASLKGSYSESTMQIILEILEKYDAKLDINEDEPDILRKGAELNDFLGVNYYASHFLKGYEGETEIYHNGTGKKGTSIFRIKGVGERVKNPEIETTDWDWPIYPKGLYDMLVRIKNEYPDCQKLYVTENGMGYKDEFINGKIEDIPRIDYIKKHLAAINQAITAGVNVKGYFVWSLMDVLSWTNGFNKRYGLFYVDFQTQKRYPKKSAYWYKETAESKVIK

SEQ ID NO: 42 - protein, 474 aa, Sebaldella termitidis
MRKQLPKDFVIGGATAAYQVEGATKEDGKGRVLWDDFLEKQGRFSPDPAADFYHRYDEDLALAEAYGHQVIRLSIAWSRIFPDGAGAVEPRGVAFYHRLFAACAKHHLIPFVTLHHFDTPERLHAIGDWLSQEMLEDFVEYARFCFEEFPEIKHWITINEPTSMAVQQYTSGTFPPAETGHFDKTFQAEHNQIVAHARIVNLYKSMGLDGEIGIVHALQTPYPYSDSSEDQHAADLQDALENRLYLDGTLAGDYAPKTLALIKEILAANQQPMFKYTDEEMAAIKKAAHQLDFVGVNNYFSKWLRAYHGKSETIHNGDGSKGSSVARLHGIGEEKKPAGIETTDWDWSIYPRGMYDMLMRIHQDYPLVPAIYVTENGIGLKESLPAEVTPNTVIADPKRIDYLKKYLSAVADAIQAGANVKGYFVWSLQDQFSWTNGYSKRYGLFFVDFPTQKRYVKQSAEWLKQVSQTHVIPE

SEQ ID NO: 43 - protein, 479 aa, Bacillus thuringiensis
MSKVIFPKGFLWGGAIAANQVEGAYVEDGKGLTTVDLLPTGENRWDIMKGNIHSFTPVEGEFYPSHEAIDFYHRYKEDIALFAEMGFKALRVSIAWTRIFPNGDDEKPNEAGLQFYDNLFDELLKHDIEPVVTMAHFDVPIHLVEKYGSWRSRKLVDFFETYAKTIFNRYKDKVKYWMTFNEINMLLHLPFMGAGLAFKEGDNKKQIQYQAAHHQLVASALAVKACHEIIPDAKIGCMLAAGATYPYTCNPDDIQRAMEQDRESFFFIDVQARGAYPGYAKRFFTDNNVTIEMEKEDEAILKEHTVDYIGFSYYASRATSTDPEVLKSITSGNVFGSVENPYLEKSEWGWTIDPKGFRITANQLYDRYQKPLFVVENGLGAIDQLNDEDEVNDAYRIDYLEKHMIEMSEAIQDGVDIIGYTSWGPIDLVSASTGEMKKRYGYIYVDKDNEGKGSLKRSKKDSFNWYKEVIATNGGSLES

SEQ ID NO: 44 - protein, 465 aa, Bacillus amyloliquefaciens
MKRFPDGFLWGGATAANQIEGAYKEGGKGLSTADVSPDGIMSPFHETDDALNLYHDAIDFYHRYQEDIALFAEMGFKAFRTSIAWTRIFPNGDETEPNEEGLQFYDRLFDELRKHQIEPVVTISHYEMPLGLVKNYGGWRNRRTVDFYERYARTVFTRYKDKVKYWMTFNEINVVLHAPFTGGGLIFREGENKQNTMYQAAHHQFVASALAVKAGHEIIPDSQIGCMIAATTTYPMTPKPEDVYAALQKERSTLFFSDVQARGSYPGYMKRFFKENGITIEMKEGDEALLKEHTVDYIGFSYYMSMTASTAPEDLAQSKGNLLGGVKNPYLKSSEWGWQIDPKGLRITLNTLYDRYQKPLFIVENGLGAVDQPEEDGSIQDDYRINYLRDHLIEAREAIEDGVDLIGYTSWGPIDLVSASTAEMKKRYGYIYVDRGNDGKGTFERKKKKSFYWYKDVIATNGESL

SEQ ID NO: 45 - protein, 478 aa, Lactococcus lactis
MTFKTDFLWGGATAANQLEGAYDIDGKGLSVADAMPGGKERLAILASPEFDWTIDTEHFTYPNHDGIDHYHHFKEDIALFAEMGFKAYRFSVAWSRIFPKGDETTPNEKGLLFYDQLIDECLKYRIEPVITISHYEMPLNLAKSYGGWKNRELIEFYVRFAKVLLERYQDKVKYWMTFNEINSATFFSGLSQGLVPSNGGDDKTNVFKAWHNQFVASAQAVKFGHDLNKNLKLGCMSIYSTTYSFDANPVNQLATQESIQEFNYFCNDVQVRGAYPAFTNRLHRKHGVNSEVLEISEEDLKIIAEGTVDYIGFSYYMSTVESKTGEGVQASGNMVLGGVKNPFLKESEWGWAIDPDGLRYALNDLYGRYQIPLFIVENGLGAIDKVEEDGTIQDDYRIDYLKKHIQSMSEAVEDGVELMGYTPWGCIDLVSASTGEMSKRYGFIYVDLDDSGNGTNKRFKKKSFDWYKQVIDSNGTNL

SEQ ID NO: 46 - protein, 470 aa, synthetic construct
MSSREKKQLSSMPNDFLWGGAISATQVEGAYNHDGKGLSNLDLALRCKKGEKRQITQQVDVNQYYPSHRAIGFYESYQKDIQLFADMGFKSLRFSIQWSRIFPTGEEERPNEAGLLFYEKILDELERHRIEPIITISHFDLPENLVTKYGSWKNRQVITFYLRFCEALFQRFSDRVRYWIPFNEINVITYMPYFSTGIHTENYQEIFQMAHHQLVASAKAVQLGRKYSSNYRFATMLMYGPTYPHNCHPESVFQAMMDDEETYYFGDIQIRGYYSPWAKKMLEQLGVQLAITEEDEQDLREGVVDFVSISYYMSWTTAPETAAGNMATGGKNPFLEQSEWGWQVDPLGLRISLNRLYQRYEKEIMIVENGLGAVDHCSENGEIYDDYRIDYLQQHLLAVKQAIVLDGVPVIGFTVWSAIDSISASTGEIGKRYGLIYVDLDDEGQGTLARKKKASFYWYQKIIESNGAEL

SEQ ID NO: 47 - protein, 455 aa, Geobacillus kaustophilus
MSQQRKSIIPDDFLWGGAVTSFQTEGAWNEGGKGLSIVDARPIPKGHSDWKVAVDFYHRYKEDIALFKELGFTAYRTSIAWTRIFPDGEGEPNEAGLAFYDAVFDELRANGIEPVITLYHFDLPLALAKKYNGFASRKVVDLFERYARTVFERYRGKVNYWLTFNEQNLVLEQPHLWGAICPEDEDPEAFAYRVCHNVFIAHAKAVKALREIAPEAKIGGMVTYLTTYPATCRPEDALANVQAKELFIDFFFDVFARGAYPRYVTNQLEKKGICLPLEAGDEELLRSQTVDFLSFSYYQSQIVRHQEQDERIIKGLEPNPYLPKTKWGWAIDPIGLRIALKDVYARYEMPIFITENGIGLEEELNENGTVDDDERIDYLRRHIEQMKMAMEEGVEVIGYLMWGATDLLSSQGEMRKRYGVIFVNRDDENLRDLKRYKKKSFYWFQRVIRTNGEEL

SEQ ID NO: 48 - protein, 470 aa, Geobacillus sp.
MKYTQLKPFPTGFLWGGSTSAYQVEGAWNEDGKGPSVIDMAKHPEGTTDFKVASDHYHRYQEDIALLAEMGFKAYRFSIAWTRIYPNGEGEVNPKGLEFYNNLINEIVRHGIEPIVTIYHFDLPYALQTKGGWSNRATIDAFVNYCRTLFEHFGDRVKYWLTINEQNMMILHGEAIGIVDPDSENPKKELYQQNHHMFVAQAKAMALCHEMLPDAKIGPAPNIATIYPASSKPEDVLAANTYSAIRNWLYLDMAVYGRYNPTAWAYLEEKGYTPTIADGDMDILQNAKPDFIAFNYYTSQTVAASVGNESDIGHTGDQHITIGEPGVYKGASNPNLPKNDFGWEIDPIGFRTTLREIYERYRLPLIVTENGLGAYDRLEEGDIVNDTYRIDFLRNHIEQMRLAITDGVDVFGYCPWSAIDLVSTHQGISKRYGFIYVNRDEFDLKDLRRIRKQSFYWYQRVISSNGEQLD

SEQ ID NO: 49 - protein, 478 aa, Geobacillus kaustophilus
MEHRHLKPFPPGFLWGAASAAYQVEGAWNEDGKGLSVWDVFAKQPGRTFKGTNGDVAVDHYHRYKEDVALMAEMGLKAYRFSVSWSRVFPDGNGAVNEKGLDFYDRLIEELRTHGIEPIVTLYHWDVPQALMDAYGAWESRRIIDDFDRYAVTLFQRFGDRVKYWVTLNEQNIFISLGYRLGLHPPGVKDMKRMYEANHIANLANAKVIQSFRHYVPDGKIGPSFAYSPMYPYDSRPENVLAFENAEEFQNHWWMDVYAWGMYPQAAWNYLESQGLEPTVAPGDWELLQEAKPDFMGVNYYQTTTVEHNPPDGVSEGVMNTTGKKGTSTSSGIPGLFKTVRNPYVDTTNWDWAIDPVGLRIGLRRIANRYRLPILITENGLGEFDTLEPDDIVNDDYRIDYLRRHIQEIQRAITDGVDVLGYCVWSFTDLLSWLNGYQKRYGFVYVNRDDESEKDLRRIKKKSFYWYQRVIATNGAEL

SEQ ID NO: 50 - protein, 462 aa, unidentified
MSAAKLPKSFVWGYATAAYQIEGSPDKDGREPSIWDTFCKAPGKIADGSSGDVATDSYNRWREDVQLLKSYGVKAYRFSLSWSRIIPKGGRSDPVNGAGIKHYRTLIEELVKEGITPFVTLYHWDLPQALDDRYGGWLNKEEAIQDFTNYAKLCFESFGDLVQNWITFNEPWVISVMGYGNGIFAPGHVSNTEPWIVSHHIILAHAHAVKLYRDEFKEKQGGQIGITLDSHWLIPYDDTDASKEATLRAMEFKLGRFANPIYKGEYPPRIKKILGDRLPEFTPEEIELVKGSSDFFGLNTYTTHLVQDGGSDELAGFVKTGHTRADGTQLGTQSDMGWLQTYGPGFRWLLNYLWKAYDKPVYVTENGFPVKGENDLPVEQAVDDTDRQAYYRDYTEALLQAVTEDGADVRGYFGWSLLDNFEWAEGYKVRFGVTHVDYETQKRTPKKSAEFLSRWFKEHIEE

SEQ ID NO: 51 - protein, 461 aa, Saccharophagus degradans
MKTFNPDFVWGAASSAYQVEGATTTDGRGPSIWDAFSSIPGKTYHNQNADIACDHYNRWQEDVAIMKEMGLKAYRFSISWSRIFPTGRGEVNEKGVAFYNNLIDELIKNDITPWVTLFHWDFPLALQMEMDGLLNPAIADEFANYAKLCFARFGDRVTHWITLNEPWCSAMLGHGMGSKAPGRVSKDEPYIAAHNLLRAHGKMVDIYRREFQPTQKGMIGIANNCDWREPKTDSELDKKAAERALEFFVSWFADPIYLGDYPASMRERLGERLPTFSDEDIALIKNSSDFFGLNHYTTMLAEQTHEGDVVEDTIRGNGGISEDQMVTLSKDPSWEQTDMEWSIVPWGCKKLLIWLSERYNYPDIYITENGCALPDEDDVNIAINDTRRVDFYRGYIDACHQAIEAGVKLKGYFAWTLMDNYEWEEGYTKRFGLNHVDFTTGKRTPKQSAIWYSTLIKDGGF

SEQ ID NO: 52 - protein, 469 aa, Homo sapiens
MAFPAGFGWAAATAAYQVEGGWDADGKGPCVWDTFTHQGGERVFKNQTGDVACGSYTLWEEDLKCIKQLGLTHYRFSLSWSRLLPDGTTGFINQKGIDYYNKIIDDLLKNGVTPIVTLYHFDLPQTLEDQGGWLSEAIIESFDKYAQFCFSTFGDRVKQWITINEANVLSVMSYDLGMFPPGIPHFGTGGYQAAHNLIKAHARSWHSYDSLFRKKQKGMVSLSLFAVWLEPADPNSVSDQEAAKRAITFHLDLFAKPIFIDGDYPEVVKSQIASMSQKQGYPSSRLPEFTEEEKKMIKGTADFFAVQYYTTRLIKYQENKKGELGILQDAEIEFFPDPSWKNVDWIYVVPWGVCKLLKYIKDTYNNPVIYITENGFPQSDPAPLDDTQRWEYFRQTFQELFKAIQLDKVNLQVYCAWSLLDNFEWNQGYSSRFGLFHVDFEDPARPRVPYTSAKEYAKIIRNNGLEAHL

SEQ ID NO: 53 - protein, 455 aa, Rattus norvegicus
MTVYKGGWDADGRGPCVWDTFTHQGGERVFENQTGDVACGSYTLWEEDLKCIKQLGLTHYRFSLSWSRLLPDGTTGFINQKGIDYYNKIIDDLLRNGVTPIVAIYHFDLPQALEDLGGWLSEAIVEAFDKYAQFCFSTFGDRVKQWLTINEPNILALLAYDMGIFAPGVPHIGIGGYQAAHNLIKAHARSWHSYDSLFREEQKGFVSLSLFFCWLEPADPNSAIDQEATKRAINFHLDFFAKPIFIDGDYPDVVKSQVASMSKKQGYPSSRLPEFTEEEKKMIKGTADFFAVQYYTTRLVRHQDNKKRELGFLQDVEIEFFPNPFWKNVGWIYVVPWGIRKLLKYIKDTYNNPVIYITENGFPQCDPPSLDDTQRWEYFRQTFQELFKAIHVDDVNLQLYCAWSLLDNFEWNNGYSRRFGLFHVDFEDPARPRTPYTSAKEYAKVIRNNGLAGAM

SEQ ID NO: 54 - protein, 469 aa, Castor canadensis
MAFPVGFGWGAATAAYQVEGGWDADGRGPCVWDTFTHQGGDRVFKNQTGDVACGSYTLWEEDLKCIKQLGLTHYRFSLSWSRLLPDGTTGFINQKGIDYYNKIIDDLLANGVKPIVAIYHFDLPQALEDQGGWLSEAIIEVFDKYSQFCFSTFGDRVKQWITINEPNTLATMAYDFGIFAPGVPHIGTGGYQAAHNMIKAHAKSWHSYDSLFRKEQKGMVSLSLFVCWLEPADPNSKPDQEAAKRAINFQLDFFAKPIFIDGDYPELVKSQIAYMSKKQGYPSSRLPEFTEEEKKMIKGTADFFAVQYYTSRLVKHQESNKGELGFLQDVGIEYFPDPSWKGVGWIYVVPWGIRKLLKYIKDMYNSPVIYITENGFPQCDPPSLDDTQRWEYFRQTFQELFKAIHVDKVNLQLYCAWSLLDNFEWNNGYSRRFGLFHVDFEDPARPRVPYRSAKEYAKIIKSNGLEGPL

SEQ ID NO: 55 - protein, 469 aa, Cavia porcellus
MAFPADLVGGLPTAAYQVEGGWDADGRGPCVWDTFTHQGGERVFKNQTGDVACGSYTLWEEDLKCIKQLGLTHYRFSISWSRLLPDGTTGFINQKGVDYYNKIIDDLLTNGVTPVVTLYHFDLPQALEDQGGWLSEAIIEVFDKYAQFCFSTFGNRVRQWITINEPNVLCAMGYDLGFFAPGVSQIGTGGYQAAHNMIKAHARAWHSYDSLFREKQKGMVSLSLFCIWPQPENPNSVLDQKAAERAINFQFDFFAKPIFIDGDYPELVKSQIASMSEKQGYPSSRLSKFTEEEKKMIKGTADFFAVQYYTTRFIRHKENKEAELGILQDAEIELFSDPSWKGVGWVRVVPWGIRKLLNYIKDTYNNPVIYITENGFPQDDPPSIDDTQRWECFRQTFEELFKAIHVDKVNLQLYCAWSLLDNFEWNDGYSKRFGLFHVDFEDPAKPRVPYTSAKEYAKIIRNNGLERPQ

SEQ ID NO: 56 - protein, 469 aa, synthetic construct
MAFPAGFGWGAGTAAYQIEGGWDADGRGPCVWDTFTHQGGDRIFKNQTGDVACNSYTLWEEDLKCIKQLGLTHYRFSLSWSRLLPDGTTGFINQKGVDYYNKIIDDLLKNKIIPIVTLFHFDLPQALEDRGGWLSEATIDIFDQYACFCFRTFGDRVKHWITINEANGFAILTYDLGFFAPGVPHIGTGGYQAAHNLIKAHARAWHSYNSLFRKEQKGLVSLSFFSVWLEPADPNSASDKKASERALAFELGTFAKPIFIDGDYPEVVKSQVASMSQRQGYPSSRLPEFTEEEKKMIKGTADFFAIQYYTTRLIKHKENKKGELGFLQDVEIDCSTDPSWKGENWVCVVPWGLRKLLKHVKDTYNNPVIYITENGFPQRDPPSLDDTQRWECFRQTFQELSKAIQVDKVNVQVYCAWSLLDNFEWNDGYNTRFGLYHVDFEDPARPRVPYTSAKEYAKVIRNNGLEEKP

SEQ ID NO: 57 - protein, 476 aa, Actinidia chinensis
MAQISSFNRTSFPDGFVFGIASSAYQFEGAAKEGGKGPNIWDTFTHEFPGKISNGSTGDVADDFYHRYKEDVKVLKFIGLDGFRMSISWARVLPRGKLSGGVNKEGIAFYNNVINDLLSKGIQPFITIFHWDLPQALEDEYGGFLSPHIVNDFRDFAELCFKEFGDRVKHRITMNEPWSYSYGGYDAGLLAPGRCSAFMAFCPKGNSGTEPYIVTHNLLLSHAAAVKLYKEKYQAYQKGQIGITLVTYWMIPYSNSKADKDAAQRALDFMLGWFIEPLSFGEYPKSMRRLVGKRLPRFTKEQAMLVKGSFDFLGLNYYIANYVLNVPTSNSVNLSYTTDSLSNQTAFRNGVAIGRPTGVPAFFMYPKGLKDLLVYTKEKYNDPVIYITENGMGDNNNVTTEDGIKDPQRVYFYNQHLLSLKNAIAAGVKVKGYFTWALLDNFEWLSGYTQRFGIVYVDFKDGLKRYPKDSALWFKK

SEQ ID NO: 58 - protein, 450 aa, unidentified
MAFKEGFVWGTATASYQIEGAVNEGGRGESVWDEFCRMKGKIDDDDNGDSACDSYHRYSEDIQLMKEIGIKAYRFSISWTRILPDGIGEINMEGVNYYNNLINGLLENGIEPYVTLFHWDYPMELQYKGGWLNPESPLWFENYAAICSRLFSDRVKYWITSNESQCYIGFGYGTGWHAPGFKLPVNQVVRAWHHNLKGLGLAAKAIRENAKGEVKVGLVACGEVGIPASDSEADMQAARNVLFDREHSEDSIDFGYGDLFEPALKGEYPKSLIPYLPKGWQEDMKDICVPLDFLGVNAYIGSIVEACENKKYRHLKLPVGIGKTSMEWPFKPETLYWVTRFISERYKLPVYITENGMANNDWISTDGKINDTQREDYLNQYLSALSKSIDDGADVRGYFYWSLLDNFEWAYGYAKRFGLVYVDYSNFSRTLKQSALRYKKIIELNGEVLK

SEQ ID NO: 59 - protein, 436 aa, unidentified
MTENAEKFLWGVATSAYQIEGATQEDGRGPSIWDTFARRPGAIRDGSTGEPACDHYHRYEEDIALMQSLGVGVYRFSVAWPRILPEGRGRINPKGLAFYDRLVDRLLAAGITPFLTLYHWDLPQALEDRGGWRSRETAFAFAEYAEAVARALADRVPFFATLNEPWCSAFLGHWTGEHAPGLRNLEAALRAAHHLLLGHGLAVEALRAAGARRVGIVLNFAPAYGEDPEAVDVADRYHNRYFLDPILGRGYPESPFQDPPPAPILSRDLEAIARPLDFLGVNYYAPVRVAPGTGPLPVRYLPPEGPVTAMGWEVYPEGLYHLLKRLGREVPWPLYITENGAAYPDLWTGEAVVEDPERVAYLEAHVEAALRAREEGVDLRGYFVWSLMDNFEWAFGYTRRFGLYYVDFPSQRRIPKRSALWYRERIARAQTGGSAH

SEQ ID NO: 60 - protein, 447 aa, Thermomonospora curvata
MAFTADFRWGVATAAYQIEGAVTEDGRGASVWDTFCHESGRIAGGHTGDVACDHYHRWPEDLALMADLGVDAYRFSIAWPRVQPGGRGPANPKGLDFYERLVDGLLERGITPFVTLFHWDLPQALEDAGGWLSRDTAHRFADYAALVAGRLGDRVEHWITLNEPVVVTAYGYAFGVYAPGRTLLLDALPTAHHQLLGHGLAVAALREHGRRQKIGLANHYSPAWAQDESSPADRRAAQIFDLFMNRLFTDPVLHGTLPDLSALGGPDPASYVRDGDLAAIAAPIDFLGVNYYQPTRLQAPPAGGPLPFEIVPITGHPVTGMGWPVVPDALLSLLRDLRRTHGDALPPILITENGCSYDDAPGPDGTVDDPERIDFLRAHLQAVETALAEGIDVRGYFVWSLMDNFEWSEGYGPRFGLVHIDYDTQRRTPKTSFAWYRDHIARARRTS

SEQ ID NO: 61 - protein, 472 aa, Thermobispora bispora
MTAAEQRPLAPGAFPEGFVWGTATSAYQIEGAVDADGRGPSIWDVFCRVPGAIARGESGDHACDHYHRWREDVALMSELGVGAYRFSVAWPRVLPEGAGRVEQRGLDFYRRLVDELRARDIEPFVTLYHWDLPQALEDRGGWRVRDTAERFADYAEVVAGALGDRVRYWITLNEPYCSAIAGYAEGRHAPGAREGHGALAAAHHLLLGHGLATERLRGRPGLRVGITLNMSPAVPAGPAPEDAAAARRMDLLVNRQFTDPLLGRRYPEDMAETFGAITDFSFRREGDLEIIGAPLDFLGVNYYYRIHAAAAPYEQPDPARRTAADIGARTVVPEGVRTSGLGWPVEPEGLHQTLTWLARRYPGLPPIYITENGYGDDGTLQDDGRIAYLRDHLAALADAIADGVDVRGWFCWSLLDNFEWARGYAARFGLVHVDYATQARTPKASFHWLRAFLREHAPAGPDQRSGSPSSTR

SEQ ID NO: 62 - protein, 442 aa, Deinococcus deserti
MTLTRKDFPNGFIFGTATSSYQIEGAASEDGRGPSIWDTFCRQPGRIQDGTSGDVACDHYHLWPEDLDLLRELGVDAYRFSLAWPRIQPSGSGAVNEKGLEFYDRLVDGLLERGIQPYATLYHWDLPQPLQDIGGWANREVAHHFADYAALVAGRLGDRVRSIATLNEPWCSSFLSYDIGEHAPGLRDRRLALAAAHHLLLGHGQAVQAMRALGKPAELGLVLNLTPAYPASQSAEDARATQYADGYANRWFLDPVFRGAYPQDMWDAFGQDVPDVQDGDLALIREPLDFLGVNYYTRSLVSAQGPVRPQDAEYTHMHWEVYPQGLTDLLLRLQREYPVPPMYITENGAAYPDERGHADIVHDPERLAYYQRHLAAVIEATRQGADVRGYFAWSMLDNFEWAYGYSRRFGLFYVDYQTQERTWKDSGRWFQGLMARTPVAAD

SEQ ID NO: 63 - protein, 482 aa, Cellulomonas flavigena
MTSTTRPSGRAFPADFLWGSATASYQIEGAVAEDGRAPSIWDTFSHTPGKVLDGDTGDVAVDHYHRVPQDVAIMQDLGLQAYRFSISWSRVLPAGTGEVNQAGLDFYSDLVDRLIAADIKPVVTLYHWDLPQTLEDAGGWTNRATAEAFAAYARVVARALGDRVHLWTTLNEPWCSAFLGYGSGVHAPGVTDPAAALAAVHHLNLAHGLAATAIREELGAATPVSITLNLHVTRAASPAPADVEAKRRIDTIANEVFLGPLLEGAYPERVFADTAAISDWSFVQEGDLELIRVPIDLLGVNYYSTGRVQHGTPPVGDGTPGPDGHRSSVVSPWIGADNVEWLPQPGPHTAMGWNIEPQGLVDLLLELHERYPELPLAITENGAAFYDTVTDDGRVHDPDRVAYLHDHVDAVGEARDKGVDVRGYFVWSLFDNFEWAYGYDRRFGVVHVDYDTQVRTLKDSARWYRELVRTGTIPTPESAASL

SEQ ID NO: 64 - protein, 460 aa, Bifidobacterium breve
MTMIFPKGFMFGTATAAYQIEGAVAEGGRTPSIWDTFSHTGHTLNGDTGDVADDFYHRWEDDLKLLRDLGVNAYRFSIGIPRVIPTPDGKPNQEGLDFYSRIVDRLLEYGIAPIVTLYHWDLPQYMASGDGREGGWLERETAYRIADYAGIVAKCLGDRVHTYTTLNEPWCSAHLSYGGTEHAPGLGAGPLAFRAAHHLNLAHGLMCEAVRAEAGAKPGLSVTLNLQICRGDADAVHRVDLIGNRVFLDPMLRGRYPDELFSITKGICDWGFVCDGDLDLIHQPIDVLGLNYYSTNLVKMSDRPQFPQSTEASTAPGASDVDWLPTAGPHTEMGWNIDPDALYETLVRLNDNYPGMPLVVTENGMACPDKVEVGTDGVKMVHDNDRIDYLRRHLEAVYRAIEEGTDVRGYFAWSLMDNFEWAFGYSKRFGLTYVDYESQERVKKDSFDWYRRFIADHSAR

SEQ ID NO: 65 - protein, 484 aa, Thermobifida fusca
MTSQSTTPLGNLEETPKPDIRFPSDFVWGVATASFQIEGSTTADGRGPSIWDTFCATPGKVENGDTGDPACDHYNRYRDDVALMRELGVGAYRFSIAWPRIQPEGKGTPVEAGLDFYDRLVDCLLEAGIEPWPTLYHWDLPQALEDAGGWPNRDTAKRFADYAEIVYRRLGDRITNWNTLNEPWCSAFLGYASGVHAPGRQEPAAALAAAHHLMLGHGLAAAVMRDLAGQAGRSVRIGVAHNQTTVRPYTDSEADRDAARRIDALRNRIFTEPLVKGRYPEDLIEDVAAVTDYSFVQDGDLKTISANLDMMGVNFYNPSWVSGNRENGGSDRLPDEGYSPSVGSEHVVEVDPGLPVTAMGWPIDPTGLYDTLTRLANDYPGLPLYITENGAAFEDKVVDGAVHDTERIAYLDSHLRAAHAAIEAGVPLKGYFAWSFMDNFEWALGYGKRFGIVHVDYESQTRTVKDSGWWYSRVMRNGGIFGQE

SEQ ID NO: 66 - protein, 458 aa, unidentified
MSQPRTDLAPGRFPADFTWGTATAAYQIEGAVREDGRGVSIWDRFSHTPGKTHNGDTGDVACDHYHRWQGDIELMRRLHVNAYRFSIAWPRILPEGWGRVNPPGLDFYDRLVDGLLAAGITPWVTLYHWDLPQALEDRGGWPNPDTSKAFAEYADVVTRRLGDRVKHWITLNEPWVVAFLGYFTGEHAPGRKEPESYLPVVHNLLLAHGLAVPVIRENSRDSQVGITLNLTHAYPAGDSAEDEAAAKRLDGFMNRWFLDPLFTGGYPRDMIDVFGSWVPSFDESDLGVIGAPLDFLGVNYYSPSFVQHSEGNPPLHVEQVRVDGEYTDMGWLVYPQGLYDLLTRLHRDYSPAAIVITENGAAYPDEPPVEGRVHDPKRVEYYASHLDAAQRAIRDGVPLRGYFAWSLMDNFEWAFGYSKRFGLYYVDYETLERTIKDSGLWYSRVVAEGQLVPTESVA

SEQ ID NO: 67 - protein, 444 aa, Saccharophagus degradans
MNRLTLPPSSRLRSKEFTFGVATSSYQIEGGIDSRLPCNWDTFCEQPNTIIDNTNGAIACDHINRWQDDIELIANLGVDAYRFSIAWGRVINLDGSLNNEGVTFYKNILTKLREKNLKAYITLYHWDLPQHLEDAGGWLNRDTAYKFRDYVNLITQALDDDVFCYTTLNEPFCSAYLGYEIGVHAPGIKDLASGRKAAHHLLLAHGLAMQVLRKNCPNSLSGIVLNMSPCYAGSNAQADIDAAKRADDLLFQWYAQPLLTGCYPDAINSLPDNAKPPICEGDMALISQPLDYLGLNYYTRAVFFADGNGGFTEQVPEGVELTDMGWEVYPQGLTDLLIDLNQRYTLPPLLITENGAAMVDELVNGEVNDIARINYFQTHLQAVHNAIEQGVDVRGYFAWSLMDNFEWALGYSKRFGITYVDYQTQKRTLKASGHAFAEFVSSRS

SEQ ID NO: 68 - protein, 449 aa, Vibrio vulnificus
MNKYQLPQDSQLRQADFLFGVATSSYQIEGGAQLGGRTPSIWDTFCNQPGAVDNMDNGDVACDHFHLWQQDIELIQGLGVDAYRLSMAWPRILPKDGQVNQQGLEFYERIIDECHARGLKVFVTLYHWDLPQYLEDKGGWLNRETAYKFAEYAEVVSGYFGNKIDSYATLNEPFCSAYLGYRWGIHAPGKKGEREGFLSAHHLMLAHGLAMPIMRKNAPQSMHGCVFNATPAYPYSEQDVAAAEYSDAEGFHWFIDPVLKGEYPQSVLEHQAHNMPMILDGDLDIIRGDLDFIGINFYTRCVVRFDANGELESMPQPDAEHTYIGWEIYPQALTDLLLRLKQRYPNLPPVYITENGAAGEDACINGEVNDEQRVRYFQSHLLALDEAIRAGVNVQGYFAWSLMDNFEWAYGYKQRFGIVHVDYATQKRTLKQSAIAYRNTLLARAEEKQ

SEQ ID NO: 69 - protein, 451 aa, Halothermothrix orenii
MAKIIFPEDFIWGAATSSYQIEGAFNEDGKGESIWDRFSHTPGKIENGDTGDIACDHYHLYREDIELMKEIGIRSYRFSTSWPRILPEGKGRVNQKGLDFYKRLVDNLLKANIRPMITLYHWDLPQALQDKGGWTNRDTAKYFAEYARLMFEEFNGLVDLWVTHNEPWVVAFEGHAFGNHAPGTKDFKTALQVAHHLLLSHGMAVDIFREEDLPGEIGITLNLTPAYPAGDSEKDVKAASLLDDYINAWFLSPVFKGSYPEELHHIYEQNLGAFTTQPGDMDIISRDIDFLGINYYSRMVVRHKPGDNLFNAEVVKMEDRPSTEMGWEIYPQGLYDILVRVNKEYTDKPLYITENGAAFDDKLTEEGKIHDEKRINYLGDHFKQAYKALKDGVPLRGYYVWSLMDNFEWAYGYSKRFGLIYVDYENGNRRFLKDSALWYREVIEKGQVEAN

SEQ ID NO: 70 - protein, 448 aa, unidentified
MSKITFPKDFIWGSATAAYQIEGAYNEDGKGESIWDRFSHTPGNIADGHTGDVACDHYHRYEEDIKIMKEIGIKSYRFSISWPRIFPEGTGKLNQKGLDFYKRLTNLLLENGIMPAITLYHWDLPQKLQDKGGWKNRDTTDYFTEYSEVIFKNLGDIVPIWFTHNEPGVVSLLGHFLGIHAPGIKDLRTSLEVSHNLLLSHGKAVKLFREMNIDAQIGIALNLSYHYPASEKAEDIEAAELSFSLAGRWYLDPVLKGRYPENALKLYKKKGIELSFPEDDLKLISQPIDFIAFNNYSSEFIKYDPSSESGFSPANSILEKFEKTDMGWIIYPEGLYDLLMLLDRDYGKPNIVISENGAAFKDEIGSNGKIEDTKRIQYLKDYLTQAHRAIQDGVNLKAYYLWSLLDNFEWAYGYNKRFGIVHVNFDTLERKIKDSGYWYKEVIKNNGF

SEQ ID NO: 71 - protein, 448 aa, Cohnella sp.
MASIQFPKDFVWGTATASYQIEGAYNEDGRGMSIWDTFSRTPGKVVNGDTGDIACDSYHRYEEDIALLKNLGVKAYRFSIAWPRIYPDGDGELNQKGLDYYAKVIDGLLAAGIEPCVTLYHWDLPQALQDKGGWDNRDTIRAFVRYAETAFKAFGGKVKQWITFNETWCVSFLSNYIGAHAPGNTDLQLAVNVAHNCMVAHGEAVKAFRALGISGEIGTTHNLYWFEPYTTKPEDVAAAHRNRAYNNEWFMDPTFKGQYPQFMVDWFKGKGVEVPIQPGDMETIAQPIDFIGVNFYSGGFGRYKEGEGLFDCEEVQVGFDKTFMDWNVYAEGLYKVLSWVHEEYGDVPIYITENGACYEDELTQEGRVHDAKRADYFKKHFIQCHRLIESGVPLKGYFAWSLLDNFEWAEGYVKRFGIVYTDYKTLKRYPKDSYRFIQSVIENDGFEA

SEQ ID NO: 72 - protein, 447 aa, unidentified
MSIIQFPKEMKWGVATASYQIEGAINAGGRGASIWDVFAKTPGKVKNGDNGDVACDSYHRYEEDIEIMKDLGVDMYRFSVAWPRIFPNGTGEVSREGLDYYHRLVDRLTENGIQPMCTLYHWDLPQALQEKGGWDNRDTIDAFVRYAEVMFKEFGDKINHWITFNELWCVSFLSNYIGVHAPGNTDLQLATNVAHHLLVAHGKAVQSYRKMGLDGQIGYAPNVEWNEPFSNQMEDAEACKRGNGWFIEWFMDPVFKGAYPSFLVEWFEKKGITVPIEAGDMETIQQPIDFLGINYYTGSVARYKENEGLFDLEKVDAGYEKTDIGWNIYPEGFYKVLYYITEQYGQIPIYITENGSCYNDEPVNGQVKDEGRIRYLSQHLTALKRSMESGVNIKGYMAWSLLDNFEWAEGYSMRFGIVHVNYRTLERTKKDSFYWYKQMIANQFFEL

SEQ ID NO: 73 - protein, 374 aa, unidentified
MAPHPRVQSPEYVNWTTFKANGVNLGGWLVQESTIDSQFWGTYSGGADDEWGLCEHLGSRCGPVLEHRYATYITERDIDKLASVGVGVLRIPTTYAAWIKLPGSQLYSGNQTAYLKQIADYAITKYGMHIIVDVHSLPGGTNGLTIGEASGHWGWYYNETAFDYSMQVIDAVISFVQNSGSPQSYTIEPMNEPTDNPDMSVFGTPAALSDRGATWVLKYIRAVIDRVASVNPNIPVMFQGSFKPEQYWSNQLPADANLVFDVHTYYFERNVTSETLPARLYADAQSKAGDGKFPVFTGEWAIQTLYQNSFALRERNVNAGLDAMYKYSQGSCYWTAKFSGNATVNGQGTQADYWNFEYFIDHGYIDLTRFHDTK

SEQ ID NO: 74 - protein, 414 aa, unidentified
MISNPSKSNGVKFKRGGNVAWDYENDIVRGVNLGGWFVLEPYMNPSLFEPFKNGNDESGVPVDEYHWTQTLGKETASKILEDHWAKWITEWDFQQMSNLGLNLVRIPIGYWAFQLLDNDPYVQGQVAFLDEALEWARNHNIKVWIDLHGAPGSQNGFDNSGLRDSLEFQNGDNTQVTLNVLAEIFQKYGTSDYDDVVVGIELVNEPLGPSLDMDALKKFYMDGYSSLRNTEGSVTPLIIHDAFQVSGYWDNFLTVAGGQWNVVLDHHHYQVFSAGELSRDIDQHISVACNWGWSAKNEYHWTVTGEWSAALTDCAYWLNGVNRGARWEGAYDGSPYYGSCEPYLQFSSWTDEHKTNVRRYIEAQLDAFEFTGGWIFWSWKTENAIDWDFQKLTDNGIFPQPLDDRQFPNQCGFN

SEQ ID NO: 75 - protein, 417 aa, unidentified
MITNPQNNNNNNNVKFKRGGTVAWDYDNDTIRGVNLGGWFVLEPYMNPSLFQPFSSGNGDVGIPLDEYHFTQTLGKDAASEILQKHWSTWITEDDFQQMSSLGLNFARIPIGYWAFELLSNDPYVQGQVEYLDQALEWARNSNIKVWIDLHGAPGSQNGFDNSGLRDSLQFQNGDNTQATLNALAKIFQKYGGANYSDVVIGIELLNEPLGPSLDMSALQQFFVEGYWSLRNTDGSVTPVIIHDAFQPFGYWDNFLTVANGEWNVVIDHHHYQVFSPGELSRDINQHISVACNWGWDAKKEYHWNIAGEWSAALTDCATWLNGVGRGARWEGAYDGSQYFGSCQPYLQFETWPEDYKTNVRKYVEAQLDAFEYTGGWVFWSWKTENAIEWDFQKLTANGIFPQPLTDRWYPNQCGFN

SEQ ID NO: 76 - protein, 371 aa, unidentified
MAPQAAYLDWKAFRANGVNLGGWLHQEAVIDPVWWSENGGDGIPDEWGLCAKLGRLCGPRLEQRYASYITTQDIDEMAEAGINVLRIPTGYNAWVKVPGSQLYTGNQVRFLRSISDYAIRKYGMHIIVDIHSAPGGLNGMGLGGREGGYGWFQNETALDYSFRAVDAAIAFIQSSSHPESFTLEPLNEPVDNRNMAEFGTPAALTPEGVAWVLKYFRGVLSRVQKVDARIPVMLQGSFKGEDFWSPYFAATDNIVFDVHHYYFAGRPTTSANLPEWICTDAKGAVGDGVFPVFTGEWSIQAATANTFASRALNLNTGLKVFGEYSRGSAYWTWKFSGNVPVEGEGVQGDYWSYEKFFEAGYINPSEGVSCQ

SEQ ID NO: 77 - protein, 368 aa, unidentified
MAPLASPPNSSYIDWRTFKGNGVNLGGWLEQESTIDSLFWDKYSGGASDEWGLCEHLGSQCGPVLEHRYATLITKADIDKLASGGITVLRIPTTYAAWIDLPSSQLYSGNQTAYLKEIADYAIKTYNMHIIIDTHSLPGGVNGLTIGEATGHWYWFYNETHFNYSMQVIDQVINFIQTSGSPQSYTLEPINEPADNNTNMVVFGTPLALTDHGAAWVLKYIRAVVQRVESVNPNIPVMFQGSFKYPQYWEGDFPASTNLVFDTHHYYYEHMDSSSENLPEYILADAREKSGTGKFPVFVGEWAIQATYNNTLALRKRNVLAGLETWSSFSQGSSYWTAKFTGNTSVAGQGEQKDYWCYETFIDEGYFN

SEQ ID NO: 78 - protein, 374 aa, synthetic construct
MAPHPRVQSPEYVNWTTFKANGVNLGGWLVQESTIDSQFWGTYSGGADDEWGLCEHLGSRCGPVLEHRYATYITERDIDKLASVGVGVLRIPTTYAAWIKLPGSQLYSGNQTAYLKQIADYAITKYGMHIIVDVHSLPGGVNGLTIGEASGHWGWYYNETAFDYSMQVIDAVISFVQNSGSPQSYTIEPINEPTDNPDMSVFGTPAALSDRGATWVLKYIRAVIDRVASVNPNIPVMFQGSFKPEQYWSNQLPADANLVFDVHTYYFERNVTSETLPARLYADAQSKAGDGKFPVFTGEWAIQTLYNNSFALRERNVNAGLDAMYKYSQGSCYWTAKFSGNATVNGQGTQADYWNFEYFIDHGYIDLTRFHDTK

SEQ ID NO: 79 - protein, 6 aa, synthetic construct
HHHHHH

(19) United States
(12) Patent Application Publication
(10) Pub. No.: US 2025/0122451 A1
(43) Pub. Date: Apr. 17, 2025

(54) COMPOSITIONS AND METHODS FOR HYDROLYSIS OF SMOKE-ASSOCIATED GLYCOSIDICALLY-BOUND VOLATILE PHENOLS

(71) Applicant: The Regents of the University of California, Oakland, CA (US)

(72) Inventors: Justin B. SIEGEL, Davis, CA (US); Youtian CUI, Davis, CA (US); Mary RILEY, Davis, CA (US); Marcus MORENO, Davis, CA (US); Anita OBERHOLSTER, Davis, CA (US); Mateo M. CEPEDA, Davis, CA (US)

(21) Appl. No.: 18/798,577

(22) Filed: Aug. 8, 2024

Related U.S. Application Data
(60) Provisional application No. 63/531,757, filed on Aug. 9, 2023.

(51) Int. Cl. C12G 1/022; C12N 9/24

(52) U.S. Cl. CPC C12G 1/0203; C12N 9/2402; C12Y 302/01168; C12G 2200/15

(57) ABSTRACT

The present disclosure provides compositions for hydrolyzing volatile phenols from phenolic glycosides. The disclosure also provides methods for utilizing the compositions to hydrolyze volatile phenols to remove volatile phenols from fruit products including fermented fruit products. Also provided herein are methods for measuring volatile phenols in fruit products including fermented fruit products.

### CROSS-REFERENCE

This application claims the benefit of the U.S. Provisional Application No. 63/531,757, filed Aug. 9, 2023, which application is hereby incorporated by reference in its entirety.

### REFERENCE TO A SEQUENCE LISTING

The instant application contains a Sequence Listing which has been submitted electronically in XML format and is hereby incorporated by reference in its entirety. Said XML copy, created on Aug. 26, 2024, is named 081906-1458189 253210US SL.xml and is 104,434 bytes in size.

### BACKGROUND OF THE DISCLOSURE

### Field of the Disclosure

The present disclosure generally relates to compositions and methods for hydrolyzing smoke-associated volatile phenols from phenolic glycosides, and more specifically to one or more glycosidases that have utility in hydrolyzing volatile phenols from products such as wine.

### Background Information

Many wine regions such as Australia, North America, South America, and Europe are periodically ravaged by devastating wildfires, seemingly exacerbated by prolonged droughts, intense heatwaves, and years of uncontrolled forest growth. These fires have significant detrimental impacts on wines produced from smoke-exposed fruit imparting negative smoke aromas and flavors to wine. This “smoke taint” occurs when grape berries exposed to wildfire smoke absorb the volatile phenols (VPs) produced from lignin combustion. Wines produced from these smoke-exposed grapes acquire undesirable smoky aromas, often described as ‘burnt wood’, ‘ashtray’, ‘burning rubber’, and ‘smoked meat’. These persistent aromas and flavors can be sufficiently high in concentration that resultant wines are considered unmarketable.

Due to the detrimental effect of smoke exposure on flavor, strategies to mitigate the impact of smoke taint are necessary. First, a decision must be made as to whether or not to harvest smoke-affected fruits. However, the decision to harvest the fruit may not be straightforward. Low or high concentrations of free volatile phenols and/or bound phenols glycosides may give a clear answer, intermediate levels may be difficult to interpret due to uncertainty regarding the different thresholds at which the products of the fruit become smoke tainted. In addition, small-scale fermentations take time and resources and may not be representative of the presence of volatile phenols in the final product after aging and storage.

Current methods for quantifying phenolic glycosides also present several challenges including the requirement of expensive capital equipment, limited accuracy due the molecular complexity of the glycosides, and the utilization of harsh reagents.

During wine processing and fermentation, current strategies used to mitigate smoke taint include excluding leaf material, keeping fruit cool, and minimizing the time fermentations are in contact with the skin tissue. These strategies often have limited effectiveness and are unlikely to reduce the concentration of volatile phenols below the flavor detection threshold. Methods for remediation of finished, smoke-tainted wine include treating wine with activated carbon, molecularly imprinted polymers, cyclodextrin or cellulose polymers, yeast products such as yeast lees, phenols-converting enzymes or organisms, treating with reverse osmosis or filtration, diluting wine with non-tainted wine, and adding tannins or oak chips to mask smoke sensory notes. Each of these strategies have significant challenges and limitations. Interaction with an affinity media, for example, often removes color, flavor, and desirable aroma compounds from the fermented beverages. Reverse osmosis also removes desirable aromas but also does not fully remove glycosides, resulting in the recurrence of smoke taint will return over time as the glycosides are hydrolyzed, Dilution of wine with non-tainted wine requires a high volume of non-tainted wine, and the addition of tannin or oak to the fermented beverage may produce a very different wine from the one intended.

### SUMMARY OF THE DISCLOSURE

The present disclosure provides compositions for hydrolyzing smoke associated volatile phenols from a phenolic glycoside. In one aspect, the composition includes a glucoside and/or a gentiobioside hydrolyzing enzyme with an amino acid sequence having at least 85% sequence identity to SEQ ID NO: 1-72. In one aspect, the composition includes a rutinosidase having an amino acid sequence with at least 85% sequence identity to SEQ ID NO: 73-78. In one aspect, the glucoside and/or the gentiobioside hydrolyzing enzyme is CbBg1B-1 (MBR2796233.I, SEQ ID NO: 1), OschBgIB (MBQ3381008.1, SEQ ID NO: 2), CbBg1B-2 (MBQ3268742.1, SEQ ID NO: 3), GiBg1B (SEQ ID NO: 4), TpBgIB (SEQ ID NO: 5), CrumBgl-2 (SEQ ID NO: 6), CrumBgl-7 (SEQ ID NO: 7), CrumBgl-6 (SEQ ID NO: 8), CrumBgl-8 (SEQ ID NO: 9), CrumBgl-1 (SEQ ID NO: 10), CrumBgl-4 (SEQ ID NO: 11), CrumBgl-5 (SEQ ID NO: 12), CrumBgl-3 (SEQ ID NO: 13), CbBg1B-3 (MBQ6595599,1, SEQ ID NO: 14), TeBylB (WP 088862624, SEQ ID NO: 15), VsBg/B (KJR72531, SEQ ID NO: 16), TgBglB (WP 062370819.1, SEQ ID NO: 17), TaBgIB-1 (RLG75229,1, SEQ ID NO: 18), 1aßg1B (ADM27756.1, SEQ ID NO: 19), TaBgIB-2 (RLG79985.1, SEQ ID NO: 20), CmBgIB (WP 012185712, SEQ ID NO: 21), TuBg1B (WP 013680114.1, SEQ ID NO: 22), CmBgIB (PSN97385, SEQ ID NO: 23), FcBg1B (WP 09022335S, SEQ ID NO: 24), FtBg1B (WP 069292479, SEQ ID NO: 25), FgBgIB (WP 072757753, SEQ ID NO: 26), SaciBgl (P14288, SEQ ID NO: 27), CmaqBgl (A8MBRO, SEQ ID NO: 28), TvolBgl (SEQ ID NO: 29), PfurBgl (E7FHY4, SEQ ID NO: 30), TgorBgl (SEQ ID NO: 31), FnodBgl (A7HNB8, SEQ ID NO: 32), TafrBgl (B7IGM4, SEQ ID NO: 33), LeasBgl (SEQ ID NO: 34), SequBgl (SEQ ID NO: 35), CheiBgl (C8W8S6, SEQ ID NO: 36), CaurBgl (A9WDK4, SEQ ID NO: 37), BdenBgl (SEQ ID NO: 38), SrocBgl (SEQ ID NO: 39), CaceBgl (Q97M15, SEQ ID NO: 40), SterBgl (DIAQN8, SEQ ID NO: 41), LrhaBgl (Q29ZJI, SEQ ID NO: 42), BthuBgl (SEQ ID NO: 43), BamyBgl (SEQ ID NO: 44), LlacBgl (Q9CFLO, SEQ ID NO: 45), Ent7Bg1 (SEQ ID NO: 46), GkauBg1-2 (Q5KXG4, SEQ ID NO: 47), GeoYBgl (SEQ ID NO: 48), GkauBg1-3 (QSKUY7, SEQ ID NO: 49), PchrBgl (Q25BW5, SEQ ID NO: 50), SdegBgl-1 (Q21EMI, SEQ ID NO: 51), HsapCyBgl (Q9H227, SEQ ID NO: 52), RratCyBgl (SEQ ID NO: 53), CcanCyBgl (A0A8B7TQ98, SEQ ID NO: $4), CporCyBgl (P97265, SEQ ID NO: 55), OpriCyBgl (SEQ ID NO: 56), CasinPRI (A0A2R6RAC3, SEQ ID NO: 57), CcelBgl (B8ISU2, SEQ ID NO: 58), ThonBgl (SEQ ID NO: 59), TeurBgl (DIA786, SEQ ID NO: 60), TbisBgl (D6Y5B2, SEQ ID NO: 61), DdesBgl (CICXP6, SEQ ID NO: 62), CflaBgl (DSULE7, SEQ ID NO: 63), BbreBgl (P94248, SEQ ID NO: 64), TfusBgl (SEQ ID NO: 65), TterBgl (DICGH4, SEQ ID NO: 66), SdegBgl-2 (Q21KX3, SEQ ID NO: 67), VvulBgl (Q7MG41, SEQ ID NO: 68), HoreBgl (BSCYA8, SEQ ID NO: 69), CtheBgl (P26208, SEQ ID NO: 70), BacGBgl (AOAIIOZQD8 9BACL, SEQ ID NO: 71), and/or BhalBgl (Q9KBK3, SEQ ID NO: 72). In one aspect, the glucoside and/or the gentiobioside hydrolyzing enzyme is CbBg1B-1 (MBR2796233.1; SEQ ID NO: 1). In one aspect, the rutinosidase is selected from AoryRut (SEQ ID NO: 73), CtroEXG (SEQ ID NO: 74); CmalEXG (SEQ ID NO: 75); AcreRut (SEQ ID NO: 76); and/or AniRut (SEQ ID NO: 77). In one aspect, the rutinosidase comprises the amino acid sequence of SEQ ID NO. 78. In one aspect, the composition includes the glucoside and/or the gentiobioside hydrolyzing enzyme CbBg1B-1 (MBR2796233.1; SEQ ID NO: 1); and the rutinosidase AoryRut (SEQ ID NO: 73). In one aspect, the composition includes 0.001 mg/ml to 50 mg/ml of the glucoside and/or the gentiobioside hydrolyzing enzyme. In one aspect, the composition includes about 0.01 mg/ml to 5 mg/ml of the glucoside and/or the gentiobioside hydrolyzing enzyme. In one aspect, the composition includes 0.001 mg/ml to 50 mg/ml of the rutinosidase. In one aspect, the composition includes 0.01 mg/ml to 5 mg/ml of the rutinosidase,

The present disclosure provides compositions for hydrolyzing smoke associated volatile phenols from a phenolic glycoside. In one aspect, the composition includes a glucoside and/or a gentiobioside hydrolyzing enzyme with an amino acid sequence having at least 85% sequence identity to SEQ ID NO: 1-72. In one aspect, the composition includes a rutinosidase having an amino acid sequence with a mutation at one or more of position 141, 190, 279, 307, 38, 39, 41, 87, 94, 145, 156, 168, 181, 183, 184, 214, 270, 276, 297, 324, 328, and/or 342 of SEQ ID NO: 73. In one embodiment, the mutation is at one or more of position 141, 190, and/or 279 of SEQ ID NO: 73. In one aspect, the mutation is at one or more of position 141, 190, and/or 307. In one aspect, the mutation comprises one or more of T141V, M190I,Q307N, T297V,Q38D, F39W, G4IN, G87N, T94N, T141I, T145V, Y156F, V168M, SI81Y, Q183W, S184F, T214A, N270R. L276K, R279H, M324W, S328T, and/or A342F relative to SEQ ID NO: 73. In one aspect, the mutations are T141V, M190I, and/or R279H, relative to SEQ ID NO: 73. In one aspect, the mutation comprises one or more of T141V, M190I, and/or Q307N relative to SEQ ID NO: 73 and wherein the composition comprises SEQ ID NO: 78. In one aspect, the glucoside and/or the gentiobioside hydrolyzing enzyme is ChBg1B-1 (MBR2796233.1, SEQ ID NO: 1), OschBglB (MBQ3381008.1, SEQ ID NO; 2), CbBg1B-2 (MBQ3268742.1, SEQ ID NO: 3), GiBg/B (SEQ ID NO: 4), TpBgIB (SEQ ID NO: 5), CrumBgl-2 (SEQ ID NO: 6), CrumBgl-7 (SEQ ID NO: 7), CrumBgl-6 (SEQ ID NO: 8), CrumBgl-8 (SEQ ID NO: 9), CrumBgl-1 (SEQ ID NO: 10), CrumBgl-4 (SEQ ID NO: 11), CrumBgl-5 (SEQ ID NO: 12), CrumBgl-3 (SEQ ID NO: 13), CbBg1B-3 (MBQ6595599.1, SEQ ID NO: 14), TcBgIB (WP 088862624, SEQ ID NO: 15), VxBg1B (KJR72531, SEQ ID NO: 16), TgRglB (WP 062370819.1, SEQ ID NO: 17), TaBgIB-1 (RLG75229.1, SEQ ID NO: 18), laBgIB (ADM27756.1, SEQ ID NO: 19), TaBg/B-2 (RLG79985.1, SEQ ID NO: 20), CmBgIB (WP 012185712, SEQ ID NO: 21), TuBgIB (WP 013680114.1, SEQ ID NO: 22), CmBglB (PSN97385, SEQ ID NO: 23), FcBgIB (WP 090223355, SEQ ID NO: 24), F (Bg1B (WP 069292479, SEQ ID NO: 25), FgBg1B (WP 072757753, SEQ ID NO: 26), SaciBgl (P14288, SEQ ID NO: 27), CmaqBgl (A8MBRO, SEQ ID NO: 28), TvolBgl (SEQ ID NO: 29), PfurBgl (E7FHY4, SEQ ID NO: 30), TgorBgl (SEQ ID NO: 31), FnodBgl (A7HNB8, SEQ ID NO: 32), TafiBgl (B7IGM4, SEQ ID NO; 33), LcasBgl (SEQ ID NO: 34), SequBgl (SEQ ID NO: 35), CheiBgl (C8W8S6, SEQ ID NO: 36), CaurBgl (A9WDK4, SEQ ID NO: 37), BdenBgl (SEQ ID NO: 38), SrocBgl (SEQ ID NO; 39), CaceBgl (Q97M15, SEQ ID NO: 40), SterBgl (DIAQN8, SEQ ID NO: 41), LrhaBgl (Q29ZJI, SEQ ID NO: 42), BthuBgl (SEQ ID NO: 43), BamyBgl (SEQ ID NO: 44), LlacBgl (Q9CFLO, SEQ ID NO: 45), Ent7Bg1 (SEQ ID NO: 46), GkauBg1-2 (Q5KXG4, SEQ ID NO: 47), GeoYBgl (SEQ ID NO: 48), GkauBg1-3 (Q5KUY7, SEQ ID NO: 49), PchrBgl (Q25BW5, SEQ ID NO: 50), SdegBgl-1 (Q21EMI, SEQ ID NO: 51), HsapCyBgl (Q9H227, SEQ ID NO: 52), RratCyBgl (SEQ ID NO: 53), CcanCyBgl (A0A8B7TQ98, SEQ ID NO: 54), CporCyBgl (P97265, SEQ ID NO: 55), OpriCyBgl (SEQ ID NO: 56), CasinPRI (A0A2R6RAC3, SEQ ID NO: 57), CcelBgl (B815U2, SEQ ID NO: 58), ThonBgl (SEQ ID NO: 59), TeurBgl (DIA786, SEQ ID NO: 60), ThisBgl (D6Y5B2, SEQ ID NO: 61), DdesBgl (CICXP6, SEQ ID NO: 62), CflaBgl (DSULE7, SEQ ID NO: 63), BbreBgl (P94248, SEQ ID NO: 64), TfusBgl (SEQ ID NO: 65), TterBgl (DICGH4, SEQ ID NO: 66), SdegBgl-2 (Q21KX3, SEQ ID NO: 67), VvulBgl (Q7MG41, SEQ ID NO: 68), HoreBgl (B8CY A8, SEQ ID NO; 69), CtheBgl (P26208, SEQ ID NO: 70), BacGBgl (AOAIIOZQD8 9BACL, SEQ ID NO: 71), and/or BhalBgl (Q9KBK3, SEQ ID NO: 72). In one aspect, the glucoside and/or the gentiobioside hydrolyzing enzyme is CbBg1B-1 (MBR2796233.1; SEQ ID NO: 1). In one aspect, the glucoside and/or the gentiobioside hydrolyzing enzyme is CbBg1B-1 (MBR2796233.1; SEQ ID NO: 1); and the rutinosidase comprising an amino acid sequence of SEQ ID NO: 78. In one aspect, the composition includes 0.001 mg/ml to 50 mg/ml of the glucoside and/or the gentiobioside hydrolyzing enzyme. In one aspect, the composition includes about 0.01 mg/ml to 5 mg/ml of the glucoside and/or the gentiobioside hydrolyzing enzyme. In one aspect, the composition includes 0.001 mg/ml to 50 mg/ml of the rutinosidase. In one aspect, the composition includes 0.01 mg/ml to 5 mg/ml of the rutinosidase.

The present disclosure provides isolated polypeptides having a mutation at one or more of positions 141, 190, 279, 307, 38, 39, 41, 87, 94, 145, 156, 168, 181, 183, 184, 214, 270, 276, 297, 324, 328, and/or 342 of SEQ ID NO: 73. In one aspect, the mutation is at one or more of position 141, 190, and/or 279 of SEQ ID NO: 73. In one aspect, the mutation is at one or more of position 141, 190, and/or 307 of SEQ ID NO: 73. In one aspect, the mutation includes one or more of T14IV, M190I, Q307N, T297V,Q38D, F39W, G4IN, G87N, T94N, T141I, T145V, Y156F, V168M, SI81Y, Q183W, S184F, T214A, N270R, L276K, R279H, M324W, S328T, and/or A342F relative to SEQ ID NO: 73. In one aspect, the mutation includes T141V, M190I, and/or R279H relative to SEQ ID NO: 73. In one aspect, the mutation includes one or more of T141V, M190I, and/or Q307N relative to SEQ ID NO: 73, wherein the polypeptide comprises SEQ ID NO: 78.

In one embodiment, the present disclosure provides a method of hydrolyzing smoke associated volatile phenols from phenolic glycoside in a fruit product or a fermented product. The method includes incubating the fruit product or a fermented product thereof with the composition of the disclosure. In one aspect, the fruit product or the fermented product thereof is smoke-exposed, In one aspect, the incubation is performed for about 4 hours. In one aspect, the incubation is performed at about 37 degrees C. In one aspect, the method includes removing the smoke-associated volatile phenols and/or the phenolic glycoside from the fruit product or the fermented product thereof, using filtration with activated carbon, reverse osmosis with activated carbon, yeast lees, cell walls, an enzyme, a cyclodextrin polymer and/or a molecularly imprinted polymer. In one aspect, the fruit product is derived from a fruit such as grape, an apple, a blueberry, a blackberry, a raspberry, a currant, a strawberry, a cherry, a pear, a peach, a nectarine, an orange, a pineapple, a mango, and a passionfruit. In one aspect, the fruit product is a fruit homogenate, a fruit juice, a fruit pulp, a fruit skin, a fruit peel, a fruit seed, a fruit concentrate, or combinations thereof. In one aspect, the fermented fruit product is a fermented beverage. In one aspect, the fermented beverage is table wine, dessert wine, fortified wine, sparkling wine, beer, spirits, cider, mead, liqueurs, sake, or brandy, In one aspect, the table wine is red wine, a white wine, or a rosé wine. In one aspect, the red wine is Cabernet Sauvignon, Alicante Henri Bouschet, Barbera, Bobal, Cabernet Franc, Carignan, Cinsaut, Malbec, Douce noir, Gamay, Grenache, Isabella, Merlot, Montepulciano, Mourvedre, Pinot noir, Sangiovese, Syrah, Tempranillo, Zinfandel, Aglianico, Blaufrankisch, Bordo, Carmenere, Castelão, Concord, Corvina Veronese, Criolla Grande, Croatina, Dolcetto, Dornfelder, Marufo, Mencia, Black Muscat, and/or Nebbiolo. In one aspect, the rosé wine is Provence Rosé Fresh, Grenache Rosé, Sangiovese Rosé, Syrah Rosé, Zinfandel Rosé, and/or Cabernet Sauvignon Rosé. In one aspect, the white wine is Chardonnay, Sauvignon Blanc, Pinot Grigio, Moscato, Riesling, and/or Chenin Blanc,

In one embodiment, the present disclosure provides a method of quantifying a volatile phenol and/or a phenolic glycoside in a fruit product or a fermented product thereof. In one aspect, the method includes incubating the fruit product or a fermented product thereof with the composition of the disclosure and measuring the levels of the volatile phenol and/or a phenolic glycoside using mass spectrometry. In one aspect, the mass spectrometry is gas chromatography mass spectrometry or liquid chromatography mass spectrometry. In one aspect, the fruit product or the fermented product thereof is smoke-exposed. In one aspect, the incubation is performed for about 4 hours. In one aspect, the incubation is performed at about 37 degrees C. In one aspect, the fruit product is derived from a fruit such as grape, an apple, a blueberry, a blackberry, a raspberry, a currant, a strawberry, a cherry, a pear, a peach, a nectarine, an orange, a pineapple, a mango, and a passionfruit. In one aspect, the fruit product is a fruit homogenate, a fruit juice, a fruit pulp, a fruit skin, a fruit peel, a fruit seed, a fruit concentrate, or combinations thereof. In one aspect, the fermented fruit product is a fermented beverage. In one aspect, the fermented beverage is table wine, dessert wine, fortified wine, sparkling wine, beer, spirits, cider, mead, liqueurs, sake, or brandy. In one aspect, the table wine is red wine, a white wine, or a rose wine. In one aspect, the red wine is Cabernet Sauvignon, Alicante Henri Bouschet, Barbera, Bobal, Cabernet Franc, Carignan, Cinsaut, Malbec, Douce noir, Gamay, Grenache, Isabella, Merlot, Montepulciano, Mourvedre, Pinot noir, Sangiovese, Syrah, Tempranillo, Zinfandel, Aglianico, Blaufrankisch, Bordo, Carmenere, Castelão, Concord, Corvina Veronese, Criolla Grande, Croatina, Dolcetto, Dornfelder, Marufo, Mencia, Black Muscat, and/or Nebbiolo. In one aspect, the rosé wine is Provence Rosé Fresh, Grenache Rosé, Sangiovese Rosé, Syrah Rosé, Zinfandel Rosé, and/or Cabernet Sauvignon Rosé, In one aspect, the white wine is Chardonnay, Sauvignon Blanc, Pinot Grigio, Moscato, Riesling, and/or Chenin Blanc.

In one embodiment, the present disclosure provides a cell engineered to express (i) a glucoside and/or a gentiobioside hydrolyzing enzyme having an amino acid sequence with at least 85% sequence identity to SEQ ID NO: 1-72; and/or a rutinosidase having an amino acid sequence with at least 85% sequence identity to SEQ ID NO: 73-77. In one aspect, the cell expresses the glucoside and/or the gentiobioside hydrolyzing enzyme CbBg1B-1 (MBR2796233.1; SEQ ID NO: 1); and the rutinosidase AoryRuf (SEQ ID NO: 73).

In one embodiment, the present disclosure provides a cell engineered to express a polypeptide with a mutation at one or more of position 141, 190, 279, 307, 38, 39, 41, 87, 94, 145, 156, 168, 181, 183, 184, 214, 270, 276, 297, 324, 328, and/or 342 of SEQ ID NO: 73.

In one embodiment, the present disclosure provides a cell engineered to express (i) a glucoside and/or a gentiobioside hydrolyzing enzyme having an amino acid sequence with at least 85% sequence identity to SEQ ID NO: 1-72; and/or a rutinosidase with a mutation at one or more of position 141, 190, 279, 307, 38, 39, 41, 87, 94, 145, 156, 168, 181, 183, 184, 214, 270, 276, 297, 324, 328, and/or 342 of SEQ ID NO: 73. In one aspect, the mutation is at one or more of position 141, 190, and/or 279 of SEQ ID NO: 73. In one aspect, the mutation is at one or more of position 141, 190, and/or 307 of SEQ ID NO: 73. In one aspect, the mutation includes one or more of T141V, M190I, Q307N, T297V,Q38D, F39W, G4IN, G87N, T94N, T141I, TI4SV, YIS6F, V168M, S181Y, Q183W, $184F, T214A, N270R, L276K, R279H, M324W, S328T, and/or A342F relative to SEQ ID NO: 73, In one aspect, the mutation includes T141V. M190I, and/or R279H relative to SEQ ID NO: 73. In one aspect, the mutation includes one or more of T141V, M190I, and/or Q307N relative to SEQ ID NO: 73. In one aspect, the rutinosidase includes an amino acid sequence of SEQ ID NO: 78.

The present disclosure also provides methods of hydrolyzing smoke-associated phenols from phenolic glycoside from a fruit fermentation apparatus and/or a fruit fermentation container. In one aspect, the method includes incubating the fruit fermentation apparatus and/or the fruit fermentation container with the composition or polypeptides described herein. In one aspect, the fruit fermentation apparatus and/or the fruit fermentation container can be a crusher, a destemmer, a fermentation vessel, a press, a pump, an airlock, a fermentation lock, a hydrometer, a refractometer, a thermometer, a primary fermenter, a secondary fermenter, a bottle, a barrel, a demijohn, a keg, a fermentation bucket, or a cork.

The present disclosure also provides for methods resulting in compositions having levels (e.g., elevated levels) of smoke-associated volatiles products as described herein, as well as compositions resulting from the methods described herein. In some embodiments, the composition comprises a fruit-derived beverage (e.g., as described herein) and levels (e.g., elevated levels) of smoke-associated volatiles (e.g., compared to starting levels in smoke-associated fruit): guaiacol, 4-methylguaiacol, 4-ethylguaiacol, p-cresols, m-cresols, o-cresols, phenol, 4-ethylphenol, syringol, and/or 4-methylsyringol, at levels above 37.0 μg/L (e.g., up to 50, 100, or 200 μg/L), 6.2 ρg/L (e.g., up to 20, 50, 100, or 200 ρg/L), 0.5 μg/L (e.g., up to 10, 50, 100, or 200 μg/L), 16,3 ρg/L (e.g., up to 50, 100, or 200 μg/L), 26.2 ρg/L (e.g., up to 50, 100, or 200 g/L), 23.5 ρg/L (e.g., up to 50, 100, or 200 μg/L), 79.1 g/L (e.g., up to 100 or 200 μg/L), 6.2 ag/L (e.g., up to 20, 50, 100, or 200 ρg/L), 51.2 μg/L (e.g., up to 100 or 200 μg/L), 4.1 μg/L (e.g., up to 10, 20, 50, 100, or 200 μg/L), respectively. In some embodiments, the disclosure provides a composition comprising a fruit-derived beverage and having levels of smoke-associated volatiles from: guaiacol, 4-methylguaiacol, 4-ethylguaiacol, p-cresols, m-cresols, o-cresols, phenol, 4-ethylphenol, syringol, and/or 4-methylsyringol, at levels above 2.2 ρg/L (e.g., up to 10, 25, 50, 100, or 200 μg/L), 0.3 μg/L (e.g., up to 10, 25, 50, 100, or 200 μg/L), 0.1 μg/L (e.g., up to 10, 25, 50, 100, or 200 g/L), 1.1 μg/L (e.g., up to 10, 25, 50, 100, or 200 μg/L), 1.1 μg/L (e.g., up to 10, 25, 50, 100, or 200 μg/L), 1.6 ρg/L (e.g., up to 10, 25, 50, 100, or 200 μg/L), 7.4 μg/L (e.g., up to $10, 25, 0, 100, or 200 μg/L), 0,3 μg/L (e.g., up to 10, 25, 50, 100, or 200 ρg/L), 31.1 μg/L (e.g., up to 50, 100, or 200 μg/L), 0.3 μg/L (e.g., up to 10, 25, 50, 100, or 200 μg/L), respectively.

In some embodiments, the pH of the beverage is between 2-5 (e.g., 2.5-4.0, 2.8-4.0 or 3.0-4.0).

### BRIEF DESCRIPTION OF THE DRAWINGS

FIG. 1A is a picture showing sequence similarity network (SSN) of GHI enzyme family. FIG. 1B is a graph showing the utilization of LC-MS analysis for activity screening in wine.

FIG. 1C is a picture showing the preliminary screening of active GHI on compound 1a. FIG. 1D is a picture of a semi-quantitative heatmap of the degrees of conversion by GHI enzymes on 1a and 1b in buffer and wine.

FIG. 2A is a picture showing the activity profiles of three candidates. ChBg1B-1 was the only candidate capable of using phenol rutinoside. FIG. 2B is a picture showing the ability of CbBgIB to convert phenolic glycosides. FIG. 2C is a graph showing the protein concentrations of the candidates. FIG. 2D is a picture showing relative efficacy of ChBg1B-1 catalyzed hydrolysis compared to acid hydrolysis in the matrix of smoke-impacted Cabernet Sauvignon. FIG. 2E is a graph showing the ability of ChBg1B-1 to convert phenolic glycosides. FIG. 2F is a graph showing the relative efficacy of CbBgB-1 catalyzed hydrolysis compared to acid hydrolysis.

FIG. 3A is a picture showing SSN of GH5 enzyme family. FIG. 3B is a picture of a semi-quantitative heatmap of the degrees of conversion by rutinosidase candidates on 2c in buffer and wine. FIG. 3C is a graph showing utilization of LC-MS analysis for rutinosidase screening in wine.

FIG. 3D is a graph showing fortification experiment involving AoryRut and enzyme cocktail of CbGgIB-1 and AoryRut against various glycosides fortified into a baseline wine,

FIG. 4A is a graph showing optimization of optimization of reaction duration. FIG. 4B is a graph showing optimization of loading concentration of CbGgIB-1 in smoke-impacted wine.

FIG. 4C is a graph showing optimization of loading concentration of AoryRut in smoke-impacted wine. P<0.05 denotes significant difference; NS denotes not significant. FIG. 4D is a graph comparing enzymatic and acid hydrolysis in Cabernet Sauvignon wine and Cabernet Sauvignon grape with different levels of smoke impact. FIG. 4E is a table showing concentration of free VPs and total VPs after two hydrolysis methods. The unit for wine is μg/L and for berry is μg/kg. The values are expressed as the average #standard deviation. FIG. 4F is a picture showing relative efficacy of enzymatic hydrolysis to acid hydrolysis for each bound VP in wine. FIG. 4G is a picture showing relative efficacy of enzymatic hydrolysis to acid hydrolysis for each bound VP in grape berries. FIG. 4H represents box and whisker plots of relative efficacy of enzymatic hydrolysis to acid hydrolysis for VP glycosides (median (line), mean (X)). FIG. 4I is a bar graph showing individual volatile phenols (VP) concentration before (free) and after enzymatic hydrolysis of high smoke-impacted wine. FIG. 4J is a bar graph showing the sum of VPs concentration before (Free) and after enzymatic hydrolysis of high smoke-impacted wine; Rapidase=DSM Rapidase Revelation Aroma with final concentration of 0.03 g/L in samples.** denotes statistically significant with p-value <0.05. FIG. 4K is a table showing the quantification results of VP glycosides through LC-MS/MS in a spike-recovery experiment.

FIG. 5 shows enzymatic activity of AoryRut mutant MC56 (SEQ ID NO: 78).

### DETAILED DESCRIPTION

The present disclosure provides compositions and methods for hydrolyzing volatile phenols from phenolic glycosides. Specifically, certain glucosidases, gentiobiosidases and rutinosidases and combinations thereof hydrolyze smoke associated volatile phenols from phenolic glycosides. Further, novel methods of quantifying levels of volatile phenols are disclosed.

When fruits such as grapes are exposed to wildfire smoke, certain smoke-related volatile phenols (VPs) can be transferred into the fruit. Once inside the fruit, the VPs can be converted into phenolic glycosides through glycosylation. These phenolic glycosides can be particularly problematic from a winemaking standpoint as they can lead to defects in aroma and flavor. Current methods for quantifying and/or eliminating these phenolic glycosides present several challenges including the requirement of expensive capital equipment, limited accuracy due the molecular complexity of the glycosides, and the utilization of harsh reagents. There is therefore a need in the art for composition and methods for hydrolyzing smoke-related phenolic glycosides to facilitate both their quantification and removal from wines.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein may be used in the practice or testing of the invention, it will be understood that modifications and variations are encompassed within the spirit and scope of the instant disclosure. The preferred methods and materials are now described.

As used in this specification and the appended claims, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value may vary up or down by 5-10%. For example, for a value of about 100, means 90 to 110 (or any value between 90 and 110).

In some embodiments, the present disclosure provides compositions for hydrolyzing smoke-associated phenolics from a phenolic glycoside.

In some embodiments, volatile phenolics are produced from lignin combustion in wildfires. Such volatile phenolics can be absorbed by fruit exposed to wildfire smoke. In some embodiments, hydrolysis of a non-volatile phenolic glycoside results in the production of one or more volatile phenols. In some embodiments, the fruit is grape berries.

Non-limiting examples of volatile phenols include, guaiacol (also herein VP1), 4-methylguaiacol (also herein VP2), 4-ethylguaiacol (VP3), cresol-p (VP4), cresol-m (VP5), cresol-o (VP6), phenol (VP7), 4-ethylphenol (VP-8), syringol (VP-9), and/or 4-methylsyringol (VP-10).

As used herein, the term “phenolic glycosides” refers to a sugar moiety bound to a phenol. In one embodiment, the phenolic glycosides are non-volatile, i.e., they are in a form that does not evaporate into a gas form under particular conditions. In one embodiment, the phenolic glycosides can be associated with smoke taint. Examples of phenolic glycoside associated with smoke taint include, without limitation, glucosides, gentiobiosides, and/or rutinosides.

In one embodiment, the phenolic glycosides can include any of the volatile phenols described herein bound to any of the glycosides described herein. In one embodiment, the phenolic glycoside is a compound of Formula I:

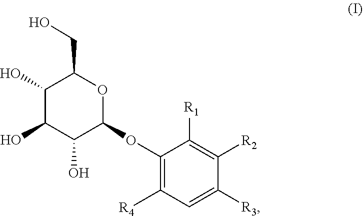

wherein R1, R2, R3 and R4 are as shown in Table 1. In one aspect, R1, R2, R3, and R4 in Formula I determine the identity of phenolic glycoside.

In one embodiment, the phenolic glycoside is a compound of Formula II:

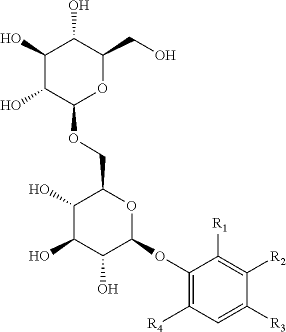

wherein R1, R2, R3 and R4 are as shown in Table 1. In one aspect, R1, R2, R3, and R4 in Formula II determine the identity of phenolic glycoside.

In one embodiment, the phenolic glycoside is a compound of Formula III:

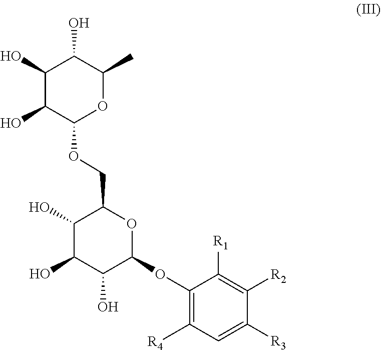

wherein R1, R2, R3 and R4 are as shown in Table 1. In one aspect, R1, R2, R3, and R4 in Formula III determine the identity of phenolic glycoside.

TABLE 1
Side chain groups of phenolic glycosides

| VPs | R1 | R2 | R3 | R4 |
|---|---|---|---|---|
| 1: guaiacol | OCH3 | H | H | H |
| 2: 4-methylguaiacol | OCH3 | H | CH3 | H |
| 3: 4-ethylguaiacol | OCH3 | H | C2H5 | H |
| 4: p-cresol | H | H | CH3 | H |
| 5: m-cresol | H | CH3 | H | H |
| 6: o-cresol | CH3 | H | H | H |
| 7: phenol | H | H | H | H |
| 8: 4-ethylphenol | H | H | C2H5 | H |
| 9: syringol | OCH3 | H | H | OCH3 |
| 10: 4-methylsyringol | OCH3 | H | CH3 | OCH3 |

In one embodiment, as described herein, compound 1a refers to guaiacol glucoside, compound 1b refers to guaiacol gentiobioside, and/or compound 1c refers to guaiacol rutinoside. In one embodiment, as described herein, compound 2a refers to 4-methylguaiacol glucoside, compound 2b refers to 4-methylguaiacol gentiobioside, and/or compound 2c refers to 4-methylguaiacol rutinoside. In one embodiment, as described herein, compound 3a refers to 4-ethylguaiacol glucoside, compound 3b refers to 4-ethylguaiacol gentiobioside, and/or compound 3c refers to 4-ethylguaiacol rutinoside. In one embodiment, as described herein, compound 4a refers to cresol-p glucoside, compound 4b refers to cresol-p gentiobioside, and/or compound 4c refers to cresol-p rutinoside. In one embodiment, as described herein, compound 5a refers to cresol-m glucoside, compound 5b refers to cresol-m gentiobioside, and/or compound 5c refers to cresol-m rutinoside. In one embodiment, as described herein, compound 6a refers to cresol-o glucoside, compound 6b refers to cresol-o gentiobioside, and/or compound 6c refers to cresol-o rutinoside. In one embodiment, as described herein, compound 7a refers to phenol glucoside, compound 7b refers to phenol gentiobioside, and/or compound 7c refers to phenol rutinoside. In one embodiment, as described herein, compound 8a refers to 4-ethylphenol glucoside, compound 8b refers to 4-ethylphenol gentiobioside, and/or compound 8c refers to 4-ethylphenol rutinoside. In one embodiment, as described herein, compound 9a refers to syringol glucoside, compound 9b refers to syringol gentiobioside, and/or compound 9c refers to syringol rutinoside. In one embodiment, as described herein, compound 10a refers to 4-methylsyringol glucoside, compound 10b refers to 4-methylsyringol gentiobioside, and/or compound 10c refers to 4-methylsyringol rutinoside.

In some embodiments, the compositions of the disclosure can hydrolyze smoke-associated volatile phenolics from one or more phenolic glycosides. In some embodiments, the compositions of the disclosure include glycosidase enzymes. In some embodiments, the compositions of the disclosure catalyze removal (release) of a glucose moiety from a glucoside associated with smoke taint. In some embodiments, the compositions of the disclosure can catalyze removal (release) of at least one glucose moiety from a gentiobioside associated with smoke taint. In some embodiments, the glycosidase can catalyze removal (release) of a glucose moiety and/or a rhamnose from a rutinoside associated with smoke taint.

In some embodiments, the glycosidase is a glycosidase 1 (GHI) enzyme. In some embodiments, GHIs catalyze the hydrolysis of β1-4 bonds. In some embodiments, the glycosidase is glycosidase derived from archaea, eubacteria, and/or eukaryotes. In one embodiment, the glycosidase is derived from Oscillospiraceae bacterium, Clostridia bacterium, Thermococcus celer, Vulcanisaeta sp. AZ3, Thermococcus guaymasensis, Thermoprotei archaeon, Ignisphaera aggregans DSM 17230, Caldivirga maquilingensis, Thermoproteus uzoniensis, Candidatus Marsarchaeota G2 archaeon ECH B 3, Fervidobacterium changbaicum, Fervidobacterium thailandense, Fervidobacterium gondwanense, Sulfolobus acidocaldarius DSM 639, Vulcanisaeta distributa DSM 14429, Pyrococcus furiosus, Fervidobacterium nodosum, Thermosipho africanus, Lancefieldella parvula, Chloroflexus aurantiacus, Clostridium acetobutylicum, Sebaldella termitidis, Lactococcus lactis subsp. Lactis, Geobacillus kaustophilus, Phanerodontia chrysosporium, Homo sapiens, Castor canadensis, Cavia porcellus, Actinidia chinensis var. chinensis, Ruminiclostridium cellulolyticum, Thermomonospora curvata, Thermobispora bispora, Deinococcus deserti, Cellulomonas flavigena, Bifidobacterium breve, Thermobaculum terrenum, Saccharophagus degradans, Vibrio vulnificus, Halothermothrix orenii, Acetivibrio thermocellus, Cohnella sp. OV330, and/or Halalkalibacterium halodurans, In some embodiments, the glycosidase is a GH 5 subfamily 23 glycosidase.

In one embodiment, the glycosidase is a rutinosidase (also herein a 6-O-a-L-rhamnopyranosyl-b-D-glucosidase). In one embodiment, the rutinosidase derived from Acremonium sp, Actinoplanes missouriensis, Aspergillus niger, Candida tropicalis, Candida maltosa and/or Aspergillus oryzae RIB40.

In some embodiments, the glycosidase can be a glucoside hydrolyzing enzyme and/or a gentiobioside hydrolyzing enzyme. In one embodiment, the glucoside hydrolyzing enzyme and/or a gentiobioside hydrolyzing enzyme can include one or more enzymes from Table 2. In one embodiment, the compositions of the disclosure can include a glucoside hydrolyzing enzyme and/or a gentiobioside hydrolyzing enzyme having about 50%, 55%, 60%, 65%, 70%, 75%, 80%, 85%, 90%, 91%, 92%, 93%, 94%, 95%, 96%, 97%, 98%, 99%, or 100% to the sequences in Table 2. In one embodiment, the sequences in Table 2 can further be mutated to tune the enzymatic activity of the sequences

TABLE 2
Glucoside and/or Gentiobioside hydrolyzing enzymes

| | | | SEQ | |
|---|---|---|---|---|
| | | | ID | |
| Name | Identifiers | Organism | NO: | Sequence |
| CbBgl | MBR2796233.1 | Clostridia | 1 | MAQFPSDFIWGVACASYQCEGGWDAD |
| B-1 | | bacterium | | GKGPNIWDDFCHRAGGSTVKNNDNGD |
| | | | | VACDSYHRYPEDIALMKQHNIRAYRESI |
| | | | | SWARVMPDGDGALNEAGLAYYDDLVN |
| | | | | RLLENGIEPMVTLFHWDLPSALQYRGG |
| | | | | WLNREMVDIFARYAGVIATRFKGRVKK |
| | | | | YMTINEPQCIALGYYTDTMAPGWRCPD |
| | | | | EDVARVFHIIALAHSAAQRAIKAVDPEA |
| | | | | LVGLVPCGRLCYPREETPENIESAYRASF |
| | | | | DLTQRWAFNFNIIMDSVVLRRYDDSAPE |
| | | | | AVRRFAATIPQSDWEAMETPDFIGVNVY |
| | | | | NGTMVDAAGNDVDCYPGFPRTACKWPI |
| | | | | TPEVMHYGPMYLYRRYGLPMIISEDGLS |
| | | | | CNDIIFRDGQVHDPKRIDFLHRYLTELSR |
| | | | | AIAGGVPVKGYMQWSFLDNFEWASGY |
| | | | | DERFGLIYVDYPTLRRIPKDSARWYANV |
| | | | | IATNGACLEEG |
| OscbBglB | MBQ3381008.1 | Oscillospiraceae | 2 | MKQFPEQFLWGVACASYQCEGAWNED |
| | | bacterium | | GKGPSIWDDFCHDPAGHIRNGDTGDIAC |
| | | | | DVYHRFREDIALMKKLGIKAYRFSISWP |
| | | | | RVIPDGDGEVNEAGLRFYDELVDELLKS |
| | | | | GIEPLITLYHWDLPSALQDKGGWLNRDI |
| | | | | VAAFGRYAELIAERFRGRVRRYMTINEP |
| | | | | PCITVLGYGSGIHAPGLRLNDEKLAQIFH |
| | | | | ILALAHSEAYRRIKAVSGPETRVGIVPCG |
| | | | | RLCYPLEDTPENREAAYRATFDLSRERW |
| | | | | GFTFNIILDSLIFRRYDDSAPEAVKRFAA |
| | | | | TVPACEWEQMEKPDFIGINVYNGECVD |
| | | | | AEGKAAGRWPGFPLTATKWPVTPEVMH |
| | | | | YAPLNLSRRYGLPMMITENGQSCNDRIF |
| | | | | RDGQVHDPERIDFLHRYLLELHKAVEEG |
| | | | | APLEGYLQWSFLDNFEWSEGYGEREGIV |
| | | | | YVDYPTQRRIPKDSAFWFGRIIESNGALL |
| | | | | FSED |
| CbBglB-2 | MBQ3268742.1 | Clostridia | 3 | MVKFPSDFIWGAACAAYQCEGAWNED |
| | | bacterium | | GKGPSIWDDFCHELGNQHVNNGDSGDV |
| | | | | ACDSYHRYREDVALMKQHGLKAYRFSI |
| | | | | SWPRVIPDGDGEVNEAGLAYYDALVDA |
| | | | | LLENGIEPMITLYHWDLPSALHLKGGWQ |
| | | | | NRQIAEWFARYARIIAERFKGRVTRYMT |
| | | | | INEAQCITLLGYGIGVHAPGLKLPGEELA |
| | | | | RIYHNIALAHSAAQRAIKAVSPEAQVGF |
| | | | | VPCGNLCYPVVDTPENRDAAYRASFAY |
| | | | | TERWGFNFNIVLDSLVLRRYDDSAPAVL. |
| | | | | KKFAATIPASDWAQMEAPDFIGINVYQG |
| | | | | QPVDGEGKPVPRPAGHPLTACKWPITPP |
| | | | | VMHYGPLNVYRRYQLPIIISENGLSCND |
| | | | | VEFLDGKVHDPDRENYLHRYISELSRAI |
| | | | | QDGTPVFGYLHWSFLDNFEWNSGYDER |
| | | | | FGLIYVDYATQKRIPKDSAAWYAKVIET |
| | | | | NGACLNG |
| GfBglB | | — | 4 | MNATDCITHEPKDFIWGAACASYQCEG |
| | | | | AWNEDGKGPSIWDEFCHDTIDGKNLNIS |
| | | | | NGDIASDFYHHWREDIALMKAHNIRAY |
| | | | | RFSVSWSRVLPDGEGKVNEQGLQWYSD |
| | | | | VVDELLANGIEPMITLYHWDLPAALQD |
| | | | | KGGWLNRDIIDVFAEYAAIIAEKLKGRV |
| | | | | KRYMTLNEPACIVQAGYSKMLHAPGWR |
| | | | | VSDEKMARIFHILALSHSAAKRAIKMIDP |
| | | | | AAQVGIVTCGRLFWPERDTPENREAAY |
| | | | | RASFDLSDAYWPFKHNILLDSLIFCRYD |
| | | | | ASIPAPVRRFAATIPESDWERMETPDFIGI |
| | | | | NVYEGPCINAARETVAPMYGSPVSACR |
| | | | | WPITPEVLHYGPEYIYRRYRLPVLISENGI |
| | | | | SCNDMIFDDGRVHDPQRIQYLRRYLLAL |
| | | | | DKAIEEGTPVMGYLQWSVMDNMEWNS |
| | | | | GYNERFGMFFVDYQTKQRIPKDSAAWY |
| | | | | AKVIATNGQSLGEMPRF |
| TpBglB | | — | 5 | MALKFGKEFKFGFSTVGVQHELGLPGSE |
| | | | | FESDWIAWLRDPENIASGLVSGDDPFSG |
| | | | | PGYWHLYREDHAIAEYLGMNAAWITVE |
| | | | | WARIFPKPTTEVRAYVEQDGERITQVSL |
| | | | | EESELERLLRLANREALSHYREIMSDWK |
| | | | | SRGGFLIVNLFHWSLPLWLHDPVAVRSR |
| | | | | GPDRAPSGWLDKRTVVEFAKFAALVAR |
| | | | | ELDDLADAWYTMNEPMVVARLGYVSV |
| | | | | SSGFPPGYLSLKAYEEAKVRLAEAHARA |
| | | | | YDALREVSGKPVGLVESVSPVTVLGGES |
| | | | | SLAELVLREQLAVLDAARFGTVGGEVR |
| | | | | EDLGGRLDWVGVNYYTRVVVSPGGPLG |
| | | | | FRVESGYGYSCAPRGVSRDGRPCSDVG |
| | | | | WEVYPEGLFEAISLVSKRYGLPVYITEN |
| | | | | GVADSRDALRPSFIVSHLYQVARLLEQG |
| | | | | VDVRGYFHWNLTDNLEWAKGESPRFGL |
| | | | | VEVDYQTKKRRLRPSALVFREIALSREV |
| | | | | PYEVALAGEWS |
| CrumBgl-2 | | — | 6 | MSFTKGFLIGASTAAHQVEGNNIHSDYW |
| | | | | AQEHMPHSSFTEPSGIACDHYNRFEEDIR |
| | | | | LMAKAGLNAYRFSIEWARIEPEEGQFDE |
| | | | | SELEHYRKVVRCCRKNGIEPLITLMHFTS |
| | | | | PVWLIRQGGWEAESTVEYFRRYADFIVK |
| | | | | NLGSEIKYICTINEANMGLQLAAIAKRFQ |
| | | | | LMAQQAQKSAKNAEGTVQVGMNFQK |
| | | | | MMENMKYAAQENAEIFGTPQPQIFVSSR |
| | | | | TEQGDTLVFRAHQAAKEAIKAINPDIQV |
| | | | | GITLSLHDLQALPGGEAFAEKAWDEEFR |
| | | | | HYLPFIQDDDFLGVQNYTRTQYGPKGQ |
| | | | | MPSPENAELTQMDYEFYPEALEHVIRSV |
| | | | | HRDFKGNLIVTENGVATSDDTRRIEFIRR |
| | | | | ALQGVEHCLNDGIPVKGYCHWSLMDNF |
| | | | | EWQKGYAMTFGMIAVDRTTLKRTPKES |
| | | | | LQFLGSMIS |
| CrumBgl-7 | | — | 7 | MVKQFPPGFLWGGATAANQCEGAYDA |
| | | | | DGRGLSSVDVVPYGPERMKVSRGERKM |
| | | | | LRCEEGFSYPSHEAIDLYHHYKEDIVLFA |
| | | | | EMGFKCYRMSVAWTRILPNGDDDIPNE |
| | | | | AGLKFYEDVFRECRRYGIEPLVTIDHEDT |
| | | | | PIALIEKYGGWRDRRMIDAYIKYCTALF |
| | | | | TRYKDLVKYWITFNEINMLLHMSFMGA |
| | | | | GIYFEPGEDKEQVKYTAANNELLASARA |
| | | | | VKLAHELMPGSMVGCMLAAGQFYPYS |
| | | | | CNPADIWDGLEKDRDNYFFIDVQARGY |
| | | | | YPVWAKKRMERAGIRLELSPEDEAVLR |
| | | | | EGTVDYVAFSYYCSRCTTADPEIFEAHK |
| | | | | RPGNAVFASVENPHLPFTEWGWQIDPTG |
| | | | | LRVTINTLYDRYQKPLFVVENGMGAND |
| | | | | TLEPDGTVHDPYRIEYLRRHIEAMRDAV |
| | | | | TEDGIPLLGYTAWGCIDLVSASSGEMKK |
| | | | | RYGMIYVNKDDRGGGDLSRHRKDSFY |
| | | | | WYKKVIASNGADLD |
| CrumBgl-6 | | — | 8 | MFKEDFLWGGATAANQFEGAWDVDGK |
| | | | | GPSIPDHCTNGTRERSKLFTQTINPEYLY |
| | | | | PSHKASDFYHHYKEDIALLAEMGYKCF |
| | | | | RMSINWSRIFPTGMEKTPNEKGLEFYDK |
| | | | | VFDECRKYGIEPLVTLSHYEMPLALGVE |
| | | | | KDGWLSRETIDCFMRYVETVFARYRDK |
| | | | | VRYWITFNEINAGQMPIGDIISTGMVKG |
| | | | | YEGPINGIRRTEQERYQALHHQFVASAR |
| | | | | TVRLAHKKYPQFKVGNMLTFIAAYPVN |
| | | | | CDPDNILLAAKYMQNMNWYCSDVQVK |
| | | | | GAYPYYATAMWRDRDVILNITAKDIED |
| | | | | LENGTVDFMTFSYYMSICVGKEGEKDK |
| | | | | VSGNLTGGFKNPYLESSDWGWQIDPVGI |
| | | | | RYALNAAYDRYRIPLMIVENGLGAFDK |
| | | | | VEEDGSVHDDYRIDYMRRHIRQMKLAT |
| | | | | EDGVELMGYTNWGCIDLVSLTTGEMRK |
| | | | | RYGQVFVDKYDDGTGTLKRSRKDSFFW |
| | | | | YRNVIRTNGMEI |
| CrumBgl-8 | | — | 9 | MAKYDFPKDFNWGTATASYQVEGGAH |
| | | | | EDGKGPSIWTEFEKRPGAIFNGDNGDVA |
| | | | | SDQYHHWKEDIELMKYLGLRSYRFSMA |
| | | | | WSRVIPEGRGAINVAGLDYYKRLCDALL |
| | | | | ENGIEPYMTFYHWDLPLALQKEFGGWE |
| | | | | SRETVKYFGEYVERISKELKGRVKNYFT |
| | | | | TNEFLACSDVGYGMGSIAPGLKLPAKRL |
| | | | | NQVRHHVLLAHGTALAALRATSPEAKV |
| | | | | GLAENPWFMVPLIDTPEHVEATKLAFRE |
| | | | | ENAHFLTAIMEGKYLDCYLEKCGADAP |
| | | | | EFTDDDMKIIGGKVDLLGLNIYFGKYVC |
| | | | | KEDDKPYRIFRDDIQSTKAGRPGLYYEP |
| | | | | DAIYWGARIVTELWNVPELIVSENGTAM |
| | | | | PEDNIDVDSGRVYDLGRIKYLRNYLTSM |
| | | | | ARAISEGYPIKGYFHWSLVDNLEWNQG |
| | | | | LQPRFGLTYIDFHTLKRTLKMSGEWYRE |
| | | | | LIRTGRIV |
| CrumBgl-1 | | - | 10 | MVQFPADFTWGVACASYQCEGGWNAD |
| | | | | GKGPSIWDDFCHELNGHHVKNDDSGDV |
| | | | | ACDSYHRYREDVALMKAHNIRAYRFSIS |
| | | | | WPRVIPDGDGAVNEAGLAYYDALVDLL |
| | | | | LENGIEPMVTLYHWDLPSALQHRGGWQ |
| | | | | NRQIADWFARYADIIARRFAGRVKRYM |
| | | | | TINEAQCITELGYGRGVLAPGLQLPDEEL |
| | | | | ARIYHNIALAHSAAQRAIKAVSPDAVVG |
| | | | | FVSCGKFCFPEHDTPEAVDAAYRAMFE |
| | | | | MDEGWGFNFNVVLDSLILRRWDDSAPA |
| | | | | AVRRFVETIPPEDWDLMEAPDFVGINVY |
| | | | | NGGMVDDAGKPVPHVPGHPITACKWPI |
| | | | | TPRVMRYGPLLIHRRYGLPMIITENGLSC |
| | | | | NDIRFMDGQVHDLKRIDFLHRYLTELSK |
| | | | | AIADGAPVLGYLQWSFLDNFEWASGYD |
| | | | | ERFGIIYVDYQTLERTPKDSARWYAKVI |
| | | | | ETNGACLN |
| CrumBgl-4 | | — | 11 | MNFPKDFLWGVATSSYQIEGAEHEDGR |
| | | | | CKSVWDDFYKIPRKVVDEKSGAIACDH |
| | | | | YHRYKEDVQLIKNLGVKAYRFSVAWPR |
| | | | | IFSYDSDSRNGVVKGNLNQKGLDFYDRL |
| | | | | IDELLQNGIEPWLTLFHWDLPYELEKKG |
| | | | | GWRNRDIHHWISDYSAEIARRYSDRVTH |
| | | | | FFTLNEMPCILGGYRGWFAPGLEVNERE. |
| | | | | VFNIIHHMLLSHGSMVQAVRANAKQNV |
| | | | | LLGCAHNGLGHYPASESKEDYEAFIKA |
| | | | | MNCIEAAPGRYAPQEGSGILSGDSLTYY |
| | | | | LDPIHFGKYPDKAFELFADKMPEIKDGD |
| | | | | MKLISSPVDYQGINIYEGRPITAGSAPGK |
| | | | | KDGGWHIEPFEEGYNITAAKWPITPKSM |
| | | | | NHYFKFISDRYKKPVYVSENGMSNADIV |
| | | | | SLDGKCHDPQRIDFTERYLAELKKAIDS |
| | | | | GADVKGYFHWSLMDNYEWRNGYTERF |
| | | | | GLVHVDYQTQKRTPKDSYWWYKELVE |
| | | | | KYK |
| CrumBgl-5 | | — | 12 | MSFRKDFAWGAATAAFQIEGAWNEDG |
| | | | | KSPSIWDVFCTQPGKIEDKSDGTVACDH |
| | | | | YHRYKEDVKLMSELGLKAYRFSIAWPR |
| | | | | VIPDGRGKVNEKALDFYSNLVDELLKY |
| | | | | NITPYVTLYHWDLPYCLYLKGGWMNPE |
| | | | | ISDMFEEYTRAVAKRLGDRVKHYITFNE |
| | | | | PSVFLGCGCLEGSHAPGHKMGTRDLLN |
| | | | | MGHNVLLSHGKAVRALRELVPDAEVGI |
| | | | | TLATMPAIPVAKKNEEEAYESYFYCDKN |
| | | | | TFVWSDAFWVDPIVLGKYPEKLLSECKD |
| | | | | IFPAFTDEDMKLISQKIDFLGQNIYQGRY |
| | | | | VGEWKRPAGTAHTELSWDVFDDALEW |
| | | | | GIKHFTKRYRLPMYITENGLSCHDWVSL |
| | | | | DGKVHDPNRIDFLHRYLRGLKKAAESG |
| | | | | CDVRGYFQWSLMDNFEWAKGYNPRFG |
| | | | | MIFCDYTTQKRIPKDSAYWYKEVIETNG |
| | | | | ENL |
| CrumBgl-3 | | — | 13 | MFTRPDLPKDFLIGAATASYQVEGAANE |
| | | | | DGRTSCIWDDFAKVPGKVFQCQDGSVA |
| | | | | ADQYHRYKEDIELMAKLGFKAYRFSVS |
| | | | | WSRVLPNGGKKVNPKGIEYYRNLCIELH |
| | | | | KHNMKACCTIYHWDMPSEIQAKGGWS |
| | | | | NRQTSYELAYLAKVLFEELGDLVDMWI |
| | | | | TINEAMCITVLGYLLGIHAPGIKDKNQFI |
| | | | | RSVHHVNLAHGLVLQEYRKSGLKAPIGI |
| | | | | THNLETPRPASKDEKDRLAVQHHIALRD |
| | | | | GIFMDPIFKKAYPTYMTDELGWVFPIED |
| | | | | GDFELISQPMDFLGINYYSEHVITWSDTE |
| | | | | PFNVKEVPRWEEKMTGIGWCITPHGLLR |
| | | | | LLKWVTEYTNSTIPIYITENGCCSADKLE |
| | | | | TDPVTKQERVHDTQRVRYLSDHLNICAE |
| | | | | AIKNNIPLKGYFCWSFIDNYEWTYGYSM |
| | | | | RFGLVYCDYQTQRRIPKDSAYFMRDVM |
| | | | | AGYGD |
| ChBglB-3 | MBQ6595599.1 | Clostridia | 14 | MAYFPKDFLWGVACASYQCEGGWDAD |
| | | bacterium | | GKGRNIWDDFCREPGKVKYGDTGDTAC |
| | | | | DTYHRIDEDVALMKKFGVQAYRFSLSW |
| | | | | ARILPEGDGEVNEAGLEYYSRVVDLLLE |
| | | | | NGIEPMVTLYHWDLPSALQYKGGWLNR |
| | | | | DIVKAFGRYADIVSKRFGDRVTRYMTIN |
| | | | | EPQCITALGYGKGVLAPGWVLPDVDLA |
| | | | | RIYHNIALSHSEAQRRIRGNVPGAQVGIV |
| | | | | PCGQLCYPKEETEENIEAAYRASEDLSH |
| | | | | GWWAFKFNICLDNLIRRGWDDTAPETL: |
| | | | | RRFQDTVPASDWQLMETPDFLGMNVYN |
| | | | | GDCVDGSGRNVPQPSGHPVTGCKWPVT |
| | | | | PEVLHYGPIHLYRRYQLPLYITENGLSCN |
| | | | | DVVSLDGLVHDPARIDFLHRYLRELSKA |
| | | | | LQAGIPLRGYLHWSFLDNFEWASGYDE |
| | | | | RFGLIHVDYQTLVRTPKDSAAWYRRVIE |
| | | | | TNGAEL |
| TcBglB | WP_088862624 | Thermococcus | 15 | MYKFPRDFVFGYSWSGFQFEMGLKGSE |
| | | celer | | VPNSDWWVWVHDMENIMTGLVSGDLP |
| | | | | ENGPAYWHLYSKDHDMAEKLGMDAIR |
| | | | | GGIEWARIFPEPTFDVRVTVERDEEGRIT |
| | | | | SVDVPESAIEELEKRANLEALEHYKRIYS |
| | | | | DWRERGKVFILNLYHWPLPLWLHDPIK |
| | | | | VRRFGPDRAPSGWLDDRSVVEFAKFAA |
| | | | | FVAYHLNDFVDSWSTMNEPNVVYENG |
| | | | | YGRPNSGFPPGYLSFEAVEKAKLNLIYA |
| | | | | HARAYDAIKEFSEKPVGVIYAYTWLDPL |
| | | | | SEEIAEDVRKIRENELYSFVDSVHFGESR |
| | | | | TVGEGREELKGRVDWLGVNYYSRIAFD |
| | | | | RVNGHVVPLPGYGFSGVRKGYAKSGRP |
| | | | | CSDFGWEIYPEGLEKLLRELNERYGLPM |
| | | | | MITENGMADEADRYRSYYLVSHLRAIHS |
| | | | | AIEAGADIRGYLHWSLTDNYEWAKGFQ |
| | | | | MKFGLLKVDWESKRRYIRPSALVFKEIA |
| | | | | TQKAIPEELSHLSDLRPLLQD |
| VsBglB | KJR72531 | Vulcanisaeta | 16 | MSLKFPKDFGFGFSTAGFQHEMGLPGSE |
| | | sp. AZ3 | | YESDWWVWVHDPENIAAGIVSGDLPEN |
| | | | | GPGYWHLYKSDHDIAFSLGMDTLRLGIE |
| | | | | WARVFPKPTFEVNVNADIRDGSVVSVD |
| | | | | VSEEALRRLDGLANRDAVQHYIEIIKDW |
| | | | | KDRGGKLIVNLYHWPLPLWVHDPLVVR |
| | | | | RSGPNNAPTGWLDPRTVVEFAKYAAYL |
| | | | | AWRLGEFVDMWSTMNEPNVVFSNGYL |
| | | | | YVKSGFPPGYLGIELMLRARGNLMTAH |
| | | | | ARAYDALREFSKAPIGIIYAISDVQPLTK |
| | | | | DDEEAAKAYEEAGQVSFLDAITKGSGRE |
| | | | | DLRGRLDWLGINYYSRTVVTTAKSQSSI |
| | | | | LPPARVVPGYGFACGPNAVSRDGRPCSD |
| | | | | FGWELYPEGLYNVLTRYWGRYGLPIIVT |
| | | | | ENGIADARDQWRSWFIVSHLYQLHRAL |
| | | | | GQGVDVRGYLHWNLIDNYEWASGFRM |
| | | | | KFGLVQVDYNTKKRYLRPSALVFREIAR |
| | | | | NKEIPEYLTHMIQSPTI |
| TgBglB | WP_ | Thermococcus | 17 | MWKFPKDFLFGYSWSGFQFEMGLEGSE |
| | 062370819.1 | guaymasensis | | VPNSDWWVWVHDTENIFSGLVSGHLPE |
| | | | | NGPAYWHLYKQDHDIAEGLGMEAIRGG |
| | | | | IEWARLFPKPTFDVKVDIEKDEDGNIVA |
| | | | | VDVPERAIEEMEKLADMKALEHYREIYS |
| | | | | DWKGRGKVFILNLYHWPLPLWLHDPIA |
| | | | | VRRLGPDRAPSGWLDERSVVEFVKFAA |
| | | | | FVAYHLNDLVDMWSTMNEPNVVYEQG |
| | | | | YTRPNSGFPPGYLSFESSTKAARNMAQA |
| | | | | HARAYDVIKEHSKAPVGLIYSFVWHDA |
| | | | | LNEEAEDIVKEIRKRHYEFVTAVHSGSS |
| | | | | GLLGERPDMKGKLDWIGVNYYTRVAY |
| | | | | RMNNGSIEVPPGYGYMCERGGFAKSGR |
| | | | | PASDFGWEIYPEGLENILRDLHRIYGLPM |
| | | | | MITENGIADAADRYRPYYLVSHLKAVHS |
| | | | | AMEAGADVRGYLHWSLTDNYEWAQGF |
| | | | | RMRFGLVHVDFETKKRYLRPSALAFREI |
| | | | | ATRKEIPEELSHLADLTPLMRD |
| TaBglB | RLG75229.1 | Thermoprotei | 18 | MSLKFPKDFKFGFSEAGFQFEMGLPGSE |
| | | archacon | | NPHSDWWTWVHDQENITAGIVSGDLPE |
| | | | | NGPGYWHLYQKDHEIADSLGMDSARLG |
| | | | | IEWSRLFPKPTFNIKADVEKDSAGNIISVE |
| | | | | VGEKSLEELDKIANKEAVEHYRRIFEDW |
| | | | | RKRGKLLIINLYHWPMPVWLHDPIKVRK |
| | | | | LGPDRAPAGWVDERSVVEFTKFAAYVA |
| | | | | WKLGDLPDMWSTMNEPNVVYTQGYVS |
| | | | | IKSGFPPGYLSVEASLKAAKHLIEAHARA |
| | | | | YDVLKKMTKKPVGIIYATAEIEPLTTED |
| | | | | KEIAEAAYAQHNFSFMDAIFTGTSQLVG |
| | | | | GERKDLARHLDWIGINYYSRLVVTRAK |
| | | | | TAAGWRVVEGYGFACQPRGISRAGRPC |
| | | | | SDFGWEVYPEGLYSVVKRFWERYRLPM |
| | | | | LITENGIADSVDALRPRYLVSHLAQVHK |
| | | | | LVSEGVELKGYLHWALTDNYEWAQGF |
| | | | | RMRFGLVYVDYETKK |
| IaBglB | ADM27756.1 | Ignisphaera | 19 | MGLKYPKEFIFGFSESGFQFEMGLPGSED |
| | | aggregans | | PNTDWWVWVHDPENIASTLVSGDFPEN |
| | | DSM | | GPGYWHLYRQDHDIAERLGMDGARIGI |
| | | 17230 | | EWSRIFSKPTFDVKVDVARDERGNIVYI |
| | | | | DVAEKALEELDRIANKDAVNHYREILSD |
| | | | | WKNRGKKLIINLYHWTLPLWLHDPIKV |
| | | | | RKLGIDRAPAGWVDERTVIEFVKYVAYI |
| | | | | AWKLGDLPDLWCTMNEPNVVYSIGYINI |
| | | | | KIGYPPGYLSFEAASKAMKHLVEAHAR |
| | | | | AYEVLKRFTNKPVGIIYVTTYHEPLKESD |
| | | | | RDVAEAAMYQAVEDFLDSITIGRSMSIG |
| | | | | ERKDLEKHLDWLGINYYSRLVVERYGN |
| | | | | AWRVLPGYGFACIPGGTSLAGRPCNDA |
| | | | | GWETYPEGLYIMLKRCWERYRLPIIVTE |
| | | | | NGTADAIDRLRPRYLATHLYQVWKALS |
| | | | | EGVDIRGYLHWALVDNYEWSSGFRMRF |
| | | | | GLVHVDFETKKRYLRPSALLFREIASSKE: |
| | | | | IPDEFMHMTQPQILI |
| TaBglB-2 | RLG79985.1 | Thermoprotei | 20 | MKIPKEFMLGASLSSFQFEGGERGDEDP |
| | | archaeon | | NNDWWIWVHDWENIIAGIVSGDFPENG |
| | | | | PGYWRLFRQDHDLAEKLGMNTLRVGIE |
| | | | | WSRIFPRPTFDVKVTVDKDEDGNILHVD |
| | | | | IDEKALAKLDEIADQDAVKHYIEMYSD |
| | | | | WKNRGKQLIINLYHWPLPLWIHDPIKVR |
| | | | | KYGPDRAPSGWLDEKTIIEFVKYAAYVS |
| | | | | WKLRDLADMWSTMNEPNVVYEQGYM |
| | | | | FIKNGFPPGYLSFEAAEKAKKNLIYAHA |
| | | | | RAYEVVKKITGKPVGIIYALPYIESLNGE |
| | | | | KETLEAIKSYRIYEFLDLIIKGKSVRNPIL |
| | | | | RKELASRADWLGVNYYSRIVFKFIHGKP |
| | | | | IVLQGYGFFCSSSGVSKMGLPCSDFGWE |
| | | | | IYPQGLYLLLKEIHTRYNGLPIIVTENGIS |
| | | | | DKADKLRPKYLVSHLYNTLKARNEGVP |
| | | | | VKGYLHWSLIDNYEWAQGFRQRFGLVI |
| | | | | VDFNTKKRYIRPSALVFREIALSQEIPEEL |
| | | | | MHLTHVEPLI |
| CmBglB | WP_ | Caldivirga | 21 | MIKFPSDFRFGFSTVGTQHEMGTPGSEF |
| | 012185712 | maquilingensis | | VSDWYVWLHDPENIASGLVSGDLPEHG |
| | | | | PGYWDLYKQDHSIARDLGLDAAWITIE |
| | | | | WARVFPKPTFDVKVKVDEDDGGNVVD |
| | | | | VEVNESALEELRRLADLNAVNHYRGILS |
| | | | | DWKERGGLLVINLYHWAMPTWLHDPIA |
| | | | | VRKNGPDRAPSGWLDKRSVIEFTKFAAF |
| | | | | IAHELGDLADMWYTMNEPGVVITEGYL |
| | | | | YVKSGFPPGYLDLNSLATAGKHLIEAHA |
| | | | | RAYDAIKAYSRKPVGLVYSFADYQPLR |
| | | | | QGDEEAVKEAKGLDYSFFDAPIKGELM |
| | | | | GVTRDDLKGRLDWIGVNYYTRAVLRRR |
| | | | | QDAGRASVAVVDGFGYSCEPGGVSNDR: |
| | | | | RPCSDFGWEIYPEGVYNVLMDLWRRYR |
| | | | | MPMYITENGIADEHDKWRSWFIVSHLY |
| | | | | QIHRAMEEGVDVRGYFHWNLIDNLEWA |
| | | | | AGYRMRFGLVYVDYATKRRYFRPSALV |
| | | | | MREVAKQKAIPDYLEHYIKPPRIE |
| TuBglB | WP_013680114.1 | Thermoproteus | 22 | MRKFPSGFRWGWSGAGFQFEMGLPGSE- |
| | | uzoniensis | | DPNTDWFAWVHDPENIAAGLVSGDFPE |
| | | | | NGVAYWHLYKQFHDDTVKMGLNTIRF |
| | | | | NTEWSRIFPKPTFDVRVHYEVREGRVVS |
| | | | | VDITEKALEELDKLANKDAVAHYREIFS |
| | | | | DIKSRGLYFILNLYHWPMPLWVHDPIKV |
| | | | | RRGDLSGRNVGWVAETTVVEFAKYAA |
| | | | | YVAWKFGDLADEFSTFNEPNVTYNLGFI |
| | | | | AVKAGFPPGYLSFQMARRAAVNLITAH |
| | | | | ARAYDAIRLTSKKPVGVIYAASPVYPLT |
| | | | | EADKAAAERAAYDGLWFFLDAVAKGV |
| | | | | LDGVAQDDLKGRLDWLGINYYSRSVVV |
| | | | | KRGDGYAGVPGYGFACEPNSVSRDGRP |
| | | | | TSDFGWEIYPEGLYDILTWAWRRYGLPL |
| | | | | YVTENGIADQHDRWRPYYLVSHLAQLH |
| | | | | RAIQDGVNVKGYLHWSLTDNYEWASGF |
| | | | | SKKFGLIYVDLSTKRHYWRPSAYIYREIA |
| | | | | SSNGIPDELEHLEKVPVASPEVLRGLRSL |
| CmBglB | PSN97385 | Candidatus | 23 | MISLPGIRFGWSQAGFQSEMGLPGSEDP |
| | | Marsarchaeota | | NSDWFAWVHDKENIAAGVVSGDLPEYG |
| | | G2 | | PAYWHRFREFHDAAERMELKIARIGVE |
| | | archacon | | WSRVFPKPTLDVQVDIEQRGDMVTHVD |
| | | ECH_B_3 | | VSQSQLEKMDAIASKDAVEHYRTIFSDL |
| | | | | KRRGIEFVLNLYHWPLPLWIHDPVAVRR |
| | | | | GEKTERTGWLSTRTVVEFAKFAAYISW |
| | | | | KLDDLVDAYSTMNEPNVVWGAGYTSV |
| | | | | KSGFPPGYLSFAHSSRAMYNMVQAHAR |
| | | | | AFDVLKTHKKPVGIIYANSDFQGLTAGD |
| | | | | ADVASKAEFDNRWRFFEAIVNGDLGGY |
| | | | | RDDLKGRLEWIGVNYYTRSVVRKAGEG |
| | | | | YVVVRGYGHACERNSLSADGRPTSDFG |
| | | | | WEFYPEGLGNVLVKYREKYGLPLYVTE |
| | | | | NGIADEADYQRPYYLVSHIYQVYQALR |
| | | | | RGADVKGYLHWSLADNYEWASGFTPRF |
| | | | | GLLRVDYTNKSLFWNPSAFVYKEIAGSN |
| | | | | GIPDQLEHLNRVPPTRGLRR |
| FcBglB | WP_ | Fervidobacterium | 24 | MFPNSFMFGASLSGFQFEMGNPSDPSEL |
| | 090223355 | changbaicum | | DTQTDWFVWVRDLENLLNGIVSGDLPE |
| | | | | SGAGYWKSYEKIHQLAVDFGMDTLRIGI |
| | | | | EWSRIFPSSTREIPFGEGMLEKLDSIANK |
| | | | | DAVEHYRKIMEDMKSKGLKVFVNLNHF |
| | | | | TLPLWLHDPLAVRKGKPTDKLGWVSDD |
| | | | | APVEFAKYAEYIAWKFGDIVDYWSSMN |
| | | | | EPHVVAQLGYFQILAGFPPSYFNPEWYI |
| | | | | KSLRNEATAHNLTYDAIKRHTDKPVGVI |
| | | | | YSFTWYDTLKPNNSEIFENAMWLANWN |
| | | | | FMDQVKDKVDYIGVNYYTRAMIDKLPK |
| | | | | PIEIQDFELNWYVVRGYGYACQEGGFAL |
| | | | | SGRPASEFGWEIYPEGLYYLLKAIYERY |
| | | | | NKPLIVTENGIADQNDKYRAQVLISHLY |
| | | | | AVEKAMNEGVDVRGYLHWSIVDNYEW |
| | | | | AKGYSKRFGLAYTDFEKKLYIPRPSMYV |
| | | | | FREIAKTRSIDQFKGYDPYGLMKF |
| FtBglB | WP_ | Fervidobacterium | 25 | MFPKDFMFGVSMSGFQFEMGWGDERD |
| | 069292479 | thailandens | | LDPNTDWFVWVREPGNLVNGVVSGDLP |
| | | | | EFGAGYWLNYEKIHQLAVDFGMDTIRIG |
| | | | | IEWSRIFPTSTESVDVRDPNFLDKLDELA |
| | | | | NKKAVEHYRKIMEDIKSKGLKLFVNLN |
| | | | | HFTLPLWLHDPVAVHYGRPTDKLGWVS |
| | | | | ERTVHEFAKYVAYMAKYGDIVDLWST |
| | | | | MNEPHVVSQLGYFSVSAGFPPAYFNPE |
| | | | | WYILATKHLAMAHNLGYDMIKRFSDKP |
| | | | | TGVIYSFTWYDTLNPNDREILEEAMYLT |
| | | | | NWFFMDMVKEKLDYVGVNYYTRTVID |
| | | | | RVEQPLAMGNFNVRWRILKGYGYACDE |
| | | | | GGVALSGRPASDFGWEMYPEGLYYVLK |
| | | | | AVSERYSKPIIVTENGVADWNDRLRSTH |
| | | | | LISHLYYVERALEDGIDVKGYLHWSIVD |
| | | | | NYEWAKGYSKRFGLAWTNFQTKTYHP |
| | | | | RPSMYIFRDIIRARTTKEFIGFDPYKVRTE |
| | | | | L |
| FgBglB | WP_ | Fervidobacterium | 26 | MFPKDFMFGASLSGFQFEMGNPNDPKE |
| | 072757753 | gondwanense | | VDPNTDWFVWVREPENLVNGIVSGDLP |
| | | | | EYGAGYWKNYEKVHQLAVDFGMDTLR |
| | | | | IGIEWSRVFPTSTREVPTGDGMLEALDKI |
| | | | | ANKEAVEHYRKIMEDMKSKGLKVFVNL |
| | | | | NHFTLPLWIHDPISVHKGIPTDKLGWVS |
| | | | | DDTPIEFAKYAEYIAWKFSDIVDYWSSM |
| | | | | NEPHVVAQLGYFQILAGFPPSYFRPEWYI |
| | | | | KSLVNEAKAHNLAYDAIKKYTSRPVGII |
| | | | | YSFIWYDTVNPQDRDIFENAMWLTNWY |
| | | | | YIDMVKDKADYIGINYYTRSLIDRLPAS |
| | | | | GMKFGDFELNWYPLRGYGYACPEGGM |
| | | | | SLSGRPASEFGWEVYPEGLYNLIKAIYER |
| | | | | YKKIIIVTENGIADEKDKYRSHYLISHLY |
| | | | | AVEKAMNEGANVIGYLHWSIVDNYEW |
| | | | | AKGYSKRFGLAYTDLEKKIYVPRPSMYI |
| | | | | FREIAKTKSIEQFKDYDPYKLMKF |
| SaciBgl | P14288 | Sulfolobus | 27 | MLSFPKGFKFGWSQSGFQSEMGTPGSED |
| | | acidocaldarius | | PNSDWHVWVHDRENIVSQVVSGDLPEN |
| | | DSM | | GPGYWGNYKRFHDEAEKIGLNAVRINV |
| | | 639 | | EWSRIFPRPLPKPEMQTGTDKENSPVISV |
| | | | | DLNESKLREMDNYANHEALSHYRQILE |
| | | | | DLRNRGFHIVLNMYHWTLPIWLHDPIRV |
| | | | | RRGDFTGPTGWLNSRTVYEFARESAYV |
| | | | | AWKLDDLASEYATMNEPNVVWGAGYA |
| | | | | FPRAGFPPNYLSFRLSEIAKWNIIQAHAR |
| | | | | AYDAIKSVSKKSVGIIYANTSYYPLRPQD |
| | | | | NEAVEIAERLNRWSFFDSIIKGEITSEGQ |
| | | | | NVREDLRNRLDWIGVNYYTRTVVTKAE |
| | | | | SGYLTLPGYGDRCERNSLSLANLPTSDF |
| | | | | GWEFFPEGLYDVLLKYWNRYGLPLYV |
| | | | | MENGIADDADYQRPYYLVSHIYQVHRA |
| | | | | LNEGVDVRGYLHWSLADNYEWSSGES |
| | | | | MRFGLLKVDYLTKRLYWRPSALVYREI |
| | | | | TRSNGIPEELEHLNRVPPIKPLRH |
| CmaqBgl | A8MBRO | Vulcanisaeta | 28 | MDISFPKSFRFGWSQAGFQSEMGTPGSE |
| | | distributa | | DPNTDWYVWVHDPENIASGLVSGDLPE |
| | | DSM | | HGPGYWGLYRMFHDNAVKMGLDIARI |
| | | 14429 | | NVEWSRIFPKPMPDPPQGNVEVKGNDV |
| | | | | LAVHVDENDLKRLDEAANQEAVRHYRE |
| | | | | IFSDLKARGIHFILNFYHWPLPLWVHDPI |
| | | | | RVRKGDLSGPTGWLDVKTVINFARFAA |
| | | | | YTAWKFDDLADEYSTMNEPNVVHSNG |
| | | | | YMWVKSGFPPSYLNFELSRRVMVNLIQ |
| | | | | AHARAYDAVKAISKKPIGIIYANSSETPL |
| | | | | TDKDAKAVELAEYDSRWIFFDAIIKGEL |
| | | | | MGVTRDDLKGRLDWIGVNYYSRTVVK |
| | | | | LIGEKSYVSIPGYGYGCERNSISPDGRPC |
| | | | | SDFGWEFYPEGLYDVIMKYWSRYHLPIY |
| | | | | VTENGIADAADYQRPYYLVSHIYQVYR |
| | | | | AIQEGANVKGYLHWSLTDNYEWASGFS |
| | | | | MRFGLLQVDYSTKKQYWRPSAYVYREI: |
| | | | | AKSKAIPEELMHLNTIPPTRSLRR |
| | | | | MVENNFPEDFKFGWSQSGFQSEMGYDN |
| TvolBgl | | | 29 | AMDDKSDWYVWVHDKENIQSGLVSGD |
| | | | | MPENGPGYWNNYKSFHEAAQNMGLKM |
| | | | | ARIGVEWSRLFPEPFPEKIMADAKNNSL |
| | | | | EINNNILSELDKYVNKDALNHYIEIENDI |
| | | | | KNRNIDLIINMYHWPLPVWLSDPVSVRK |
| | | | | GIKTERSGWLNDRIVQLFALFSSYIVYK |
| | | | | MEDLAVAFSTMNEPNVVYGNGFINIKSG |
| | | | | FPPSYLSSEFASKVKNNILKAHSLAYDS |
| | | | | MKKITDKPVGIIYANTYFTPLDPEKDND |
| | | | | AIAKADSDAKWSFFDPLIKGDKSLGING |
| | | | | NKLDWIGINYYTRTMLRKDGDGYISLK |
| | | | | GYGHSGSPNTVTNDKRPTSDIGWEFYPE |
| | | | | GLEYVIMNYWNRYKLPMYVTENGIADN |
| | | | | GDYQRPYYLVSHIASVLRAINKGANVK |
| | | | | GYLHWSLVDNYEWALGFSPKFGLIGYD |
| | | | | ENKKLYWRPSALVYKEIATKNCISPELK |
| | | | | HLDSIPPINGLRK |
| PfurBgl | E7FHY4 | Pyrococcus | 30 | MKFPKNFMFGYSWSGFQFEMGLPGSEV |
| | | furiosus | | ESDWWVWVHDKENIASGLVSGDLPENG |
| | | | | PAYWHLYKQDHDIAEKLGMDCIRGGIE |
| | | | | WARIFPKPTFDVKVDVEKDEEGNIISVD |
| | | | | VPESTIKELEKIANMEALEHYRKIYSDW |
| | | | | KERGKTFILNLYHWPLPLWIHDPIAVRK |
| | | | | LGPDRAPAGWLDEKTVVEFVKFAAFVA |
| | | | | YHLDDLVDMWSTMNEPNVVYNQGYIN |
| | | | | LRSGFPPGYLSFEAAEKAKFNLIQAHIGA |
| | | | | YDAIKEYSEKSVGVIYAFAWHDPLAEEY |
| | | | | KDEVEEIRKKDYEFVTILHSKGKLDWIG |
| | | | | VNYYSRLVYGAKDGHLVPLPGYGFMSE |
| | | | | RGGFAKSGRPASDFGWEMYPEGLENLL |
| | | | | KYLNNAYELPMIITENGMADAADRYRP |
| | | | | HYLVSHLKAVYNAMKEGADVRGYLHW |
| | | | | SLTDNYEWAQGFRMRFGLVYVDFETKK |
| | | | | RYLRPSALVFREIATQKEIPEELAHLADL |
| | | | | KFVTRK |
| TgorBgl | | | 31 | MYKFPRDFLFGYSWSGFQFEMGLPGSE |
| | | | | VPNSDWWAWVHDIENIAAGLVSGDLPE |
| | | | | NGPAYWDLYKKDHDIAESLGMDAIRGG |
| | | | | IEWARIFPKPTFDVKARVERDEKGNIVSV |
| | | | | EVPESSIKELEKIADMNALEHYREIYAD |
| | | | | WKERGKTFILNLYHWPLPLWLHDPLKV |
| | | | | RKLGPDRAPAGWLDDKSVVEFAKFAAF |
| | | | | VAYHLDDLVEVWSTMNEPNVVYQNGY |
| | | | | TRPTHGFPPGYLSFEAERKAKMNLIQAH |
| | | | | ARAYDVIKEYSDKDVGVIYAYTWPDPL |
| | | | | REDIEEEVRAIRERELYSFVDAVHFGKA |
| | | | | ADVEERDDLKGRVDWLGVNYYSRIAFD |
| | | | | MVNGHVLPVPGYGFSGERGGYARSGRP |
| | | | | CSDFGWEIYPEGLEQLLKDLAKRYGLPM |
| | | | | MITENGIADAADRYRPHYLVSHLKAVH |
| | | | | EAMKEGADVRGYLHWSLTDNYEWAQG |
| | | | | FRMRFGLVYVDMETKKRYLRPSALVFR |
| | | | | ELATRKEIPEELEHLSSLDFLVRR |
| FnodBgl | A7HNB8 | Fervidobacterium | 32 | MMFPKDELFGVSMSGFQFEMGNPQDAE |
| | | nodosum | | EVDLNTDWYVWVRDIGNIVNGVVSGDL |
| | | | | PENGSWYWKQYGKVHQLAADFGMDVI |
| | | | | RIGTEWSRIFPVSTQSVEYGSPDMLEKLD |
| | | | | KLANQKAVSHYRKIMEDIKAKGLKLFV |
| | | | | NLYHFTLPIWLHDPIAVHKGEKTDKIGW |
| | | | | ISDATPIEFAKYAEYMAWKFADIVDMW |
| | | | | ASMNEPHVVSQLGYFAINAGFPPSYFNP |
| | | | | SWYIKSLENEAKAHNLSYDAIKKYTNNP |
| | | | | VGVIYSFTWYDTVNKDDKESFENAMDL |
| | | | | TNWRFIDMVKDKTDYIGVNYYTRAVID |
| | | | | RLPTTIDFGEFKMNWYTLRGYGYSCEEG |
| | | | | GFSLSGRPASEFGWEIYPEGLYNILIHVY |
| | | | | NRYKKDIYVTENGIADSKDKYRSLFIISH |
| | | | | LYAIEKALNEGIPIKGYLHWSIIDNFEWA |
| | | | | KGYSKRFGLAYTDLSTKKYIPRPSMYIFR |
| | | | | EIIKDKSIDKFKGYDPYNLMKF |
| TafrBgl | B7IGM4 | Thermosipho | 33 | MFSKDFLFGASLSGFQFEMGNPNNEEEL |
| | | africanus | | DKNTDWFVWVRDLGNIINGKVSGDLPE |
| | | | | YGAGYYTNYKAVHNLAKEFGMNALRI |
| | | | | GIEWSRIFKESTKDISLDDPNMLEKLDQL |
| | | | | ADKKAIEHYRDVLEDIKSKGLVAIVNLS |
| | | | | HETLPLWLHDPINVHKGKETEKLGWVS |
| | | | | DDAPIEFAKYAEYIAWKFKDIVDMWSS |
| | | | | MNEPHVVSQLGYFQTSAGFPPSYFNPSW |
| | | | | YLKSLENQALAHNLAYDAIKKHTGKPV |
| | | | | GVIYSFTWYDTVNNDEEIFESAMFLNNW |
| | | | | NYMDRVKDKIDFVGVNYYTRAVIDRLL |
| | | | | VPIKIDNYELNWYTLSGYGYSCVEDGFA |
| | | | | NSKRPSSEIGWEIYPEGLYNILKEIYNRY |
| | | | | GKQIYITENGIADSSDKYRSFYIISHLYAV |
| | | | | EKAINEGVPVKGYLHWSIIDNYEWAKG |
| | | | | YGKRFGLAYTDFERKTYIPRPSMYILREII |
| | | | | KERSIDKFKGYDPYGLMNF |
| LcasBgl | | | 34 | MTIQFDADFVWGAATSGPQAEGTFHKK |
| | | | | HENIFDYHYHTRPQDFYHNVGPDVASNF |
| | | | | YNDYENDLALLKQAGVQALRISIQWTR |
| | | | | LIDDLEAGTVDPVGADYYRRVEKTMHQ |
| | | | | LGITPYVNLHHFDLPVTLQHQYGGWQS |
| | | | | KHVVDLYVKFATRCFELYSDQVTHWFT |
| | | | | FNEPKVIVDGQYLYQFHYPNIVDGRLAV |
| | | | | QAAYNLNLASAKAVAAFRQINRQSQGTI |
| | | | | GTIVNLTPVYPASQAPEDLAAARFAEQW |
| | | | | ANDLYLEPAIHGRFPEELVARLKRDGVL |
| | | | | WEATSDELAVIAANRIDVLGVNYYHPFR |
| | | | | VQAPAVSPDSLQAWLPDIYFDNYDMPG |
| | | | | RKMNLDKGWEIYPDALYDIAMTIKRRY |
| | | | | DNLPWFVAENGIGVANEERFLKDGMVQ |
| | | | | DDYRIQFMTDHLRFLSQAITEGANCHGY |
| | | | | FVWTGIDCWSWLNAYKNRYGLIRNDLC |
| | | | | NQTKSLKKSGHWFSQVAATGLVAPTLR |
| | | | | PFESEEKNHG |
| SequBgl | | | 35 | MKQSKRRYQFPEGFLWGSSTSGPQSEGT |
| | | | | VSGDGKGPSNWDYWESLEPDKFHHQIG |
| | | | | PEVTSTFYTNYKSDIALLKETGHTAFRTS |
| | | | | IQWSRLIPEGVGQVNPKAVAFYREVFQE |
| | | | | IMAQDIKLIVNLYHEDLPYALQGKRGWE |
| | | | | AKETVWAYETYAKTCFELFGDLVDTWI |
| | | | | TFNEPIVPVECGYLGHYHYPCKVDAKA |
| | | | | AVQVAYHTQLASSLAIKACHELYPKHRI |
| | | | | SIVLNVTPAYPRSDQPEDVKAARIAELFQ |
| | | | | TKSFLDPSVLGVYPEELVVLLEAADLLP |
| | | | | QYSADELAIIKNNPVDFLGVNYYQPLRV |
| | | | | QAPSKTRQDGEPITLASYFEPYDMPGKK |
| | | | | VNPHRGWEIYEQGLYDIALNLKEHYGNI |
| | | | | DWLVTENGMGVEGEEAFLVDGQIQDDY |
| | | | | RIAFIEDHLIQLHRALEEGANCKGYLLW |
| | | | | TFIDCWSWLNAYKNRYGLVALDLETQK |
| | | | | RTLKKSGHWFKTLSQTNGFDK |
| CbeiBgl | C8W8S6 | Lancefieldella | 36 | MQYQLPKDFFFGGAMSGPQTEGRWQD |
| | | parvula | | DGRIPSIWDTWSNLDITAFHNRVGSYGG |
| | | | | NDFSSRMEEDFELLKSIGMDSVRTSIQW |
| | | | | SRLLDIDGNLNPEGERYYHQLFATAKKV |
| | | | | GIEIFVNLYHFDMPEYLFNRGGWESREV |
| | | | | VEAYAHYARIAFETFGKEIRYWFTFNEPI |
| | | | | VEPEMRYTVGGWFPFVKNYSRARAVQY |
| | | | | NISLAHALGVREYRRAKAAGFMLEDSRI |
| | | | | GLINCFAPPYTKDNPSEADLEALRMTDG |
| | | | | VNIRWWLDLVTKGELPQDVIDTLQSRG |
| | | | | VDLPIRPEDKLILADGVVDWLGCNYYHP |
| | | | | ERIQAPAKDTDENGIPNFADPYVWPEAE |
| | | | | MNVSRGWEIYPQGLYDFAMKVRDEYPE |
| | | | | LEWFVSENGMGVEREDLKKDENGVIQD |
| | | | | DYRVDFVRRHLEWIARAIQDGAKCRGY |
| | | | | HYWAIIDNWSWANAFKNRYGFIEVDLE |
| | | | | DNYNRRLKKSAKWLKQIATTHIVD |
| CaurBgl | A9WDK4 | Chloroflexus | 37 | MQQFAFPTGFLWGAATSAHQVEGNNIN |
| | | aurantiacu | | SDSWVLEHLPDTIYAEPSGDACDYYHRY |
| | | | | PEDIALLAQLGFNAYRFSIEWARIEPEEG: |
| | | | | EFSFASLEHYRRMLATCHEHGLKPVVTL |
| | | | | HHFTSPRWLIRAGGWLDPKTPDRFVRYC |
| | | | | ERVVHYLGDLIAGACTFNEPNLPVLLSKI |
| | | | | MPASPLASPFWRAAAAEFAVTPDRLGIF |
| | | | | QFVSQPRMREIIFAAHRRAFEVLHDGPG |
| | | | | SFPVGMTLALVDIHAGPDGERMAAEFR |
| | | | | RELAEVYLEQLREDDFVGVQTYSRLVV |
| | | | | GPAGIIPPGDDVEKTQTGEEYYPEAIGGT |
| | | | | IRHAAAVAGIPVVVTENGLATTDDTRRV |
| | | | | EYFRRALRSVAECLIDGIDVRGYFAWSA |
| | | | | LDNFEWISGYKPKLGIIAVDRTTQARTPK |
| | | | | PSAYWLGNVARFNYCVED |
| BdenBgl | | | 38 | MRETYEFPQEFIWGASTAAHQIEGNNVA |
| | | | | SDWWAREHAECADLSEPSGDAADSYHR |
| | | | | YGEDIRMLADAGLGMYRFSIEWARIEPA |
| | | | | EGCFSKAQLLHYRHMIDACHENGIEPMV |
| | | | | TLNHMTLPLWLAVKGGWLNDGAVDYF |
| | | | | DRYVRYLMPILHDVTWVCTINEPNMVA |
| | | | | LTRGGTEGSDFVSASLPAPDLDISAALVE |
| | | | | AHREARGILSENPRIKSGWTIACQAFHA |
| | | | | MPGCEQEMEEYQYPREDYFTEAAAGDD |
| | | | | FIGVQAYLRTFIGKDGPVPVPEDAERTLT |
| | | | | GWEYFPPALGIAIRHTWNVAGHTPIIVTE |
| | | | | NGIATADDRRRIDYTFGAIAGMHDAMA |
| | | | | DGVDVRGYLHWSLLDNYEWGSFAPTFG |
| | | | | LACWDKDTFERHPKPSLNWLGMIAKTG |
| | | | | VMSR |
| SrocBgl | | | 39 | MTRTSLPFPDGFLWGASTAAHQIEGNNV |
| | | | | NSDWWRKEHDPAANIAEPSLDACDSYH |
| | | | | RWEQDMDLLAELGFTDYRFSVEWARIE |
| | | | | PVPGTFSHAETAHYRRMVDGALARGLR |
| | | | | PMVTLHHFTVPQWFEDLGGWTADGAA |
| | | | | DLFARYVEHCAPIIGKDVRHVCTINEPN |
| | | | | MIAVMAGLAKTGDQGFPPAGLPTPDEET |
| | | | | THAVIAAHHAAVKAVRAIDPDIQVGWTI |
| | | | | ANQVYQALPGAEDVTAAYRYPREDVFI |
| | | | | EAARGDDWIGVQSYTRTKIGADGPIPAP |
| | | | | EDAERTLTQWEYYPAAVGHALRHTADV |
| | | | | AGPDMPLIVTENGIATADDARRVDYYT |
| | | | | GALEAVSAALEDGVNIHGYLAWSALDN |
| | | | | YEWGSYKPTFGLIAVDPVTFERTAKPSA |
| | | | | VWLGEMGRTROLPRAER |
| CaceBgl | Q97M15 | Clostridium | 40 | MKFPKDFFLGAASASYQVEGAWNEDGK |
| | | acetobutylicum | | GVSNWDVFTKIPGKTFEGTNGDVAVDH |
| | | | | YHRYKEDVKLMAEMGLDSYRFSVSWP |
| | | | | RIIPDGDGEINQKGIEFYNNLIDECLKYGI |
| | | | | VPFVTLYHWDMPEVLEKAGGWTNKKT |
| | | | | VDAFVKYAKACFEAFGDRVKRWITFNE |
| | | | | TIVECSNGYLSGAHPPGITGDVKKYFQA |
| | | | | THNVETAHARSVIEYKKLKQYGEIGITH |
| | | | | VFSPAFSVDDKEENKAAAYHANQYEIT |
| | | | | WYYDPILKGKYPEYVIKNIEKQGFLPDW |
| | | | | TDEELNTLREAAPLNDFIGLNYYQPQRVI |
| | | | | KNHDTGEKIERTRENSTGAPGNASFDGF |
| | | | | YRTVKMDDKTYTKWGWEISPESLILGLE |
| | | | | KLKEQYGDIKIYITENGLGDQDPIIEDEIL |
| | | | | DMPRIKFIEAHLRAIKEAISRGINLKGYY |
| | | | | AWSVIDLLSWLNGYKKQYGFIYVDHKH |
| | | | | NLDRKKKLSFYWYKKVIEERGKNI |
| SterBgl | DIAQN8 | Sebaldella | 41. | MERLPEDFIFGAATAAFQAEGAVNEDGR |
| | | termitidis | | GKCYWDEYLHRAESTFNGDTASDFYHK |
| | | | | YREDTALCREYGINGIRISIAWTRIIPDGS |
| | | | | GKVNQKGIDFYNDMINACLEAGVEPYV |
| | | | | TLHHFDTPLELFKNGDWLNRENTEHFVR |
| | | | | FAKICFENFGDRVKKWITINEPWSVVAG |
| | | | | QYIIGHEPPNIKYDVPKAVQAMHNMCTA |
| | | | | HAKAVIEYKKMNLNGEIGIIHILESKYPIS |
| | | | | EKPEDIRAALLEDTLANKFMLDASLKGS |
| | | | | YSESTMQIILEILEKYDAKLDINEDEPDIL |
| | | | | RKGAELNDFLGVNYYASHFLKGYEGET |
| | | | | EIYHNGTGKKGTSIFRIKGVGERVKNPEI |
| | | | | ETTDWDWPIYPKGLYDMLVRIKNEYPD |
| | | | | CQKLYVTENGMGYKDEFINGKIEDIPRID |
| | | | | YIKKHLAAINQAITAGVNVKGYFVWSL |
| | | | | MDVLSWTNGFNKRYGLFYVDFQTQKR |
| | | | | YPKKSAYWYKETAESKVIK |
| LrhaBgl | Q29ZJ1 | Sebaldella | 42 | MRKQLPKDFVIGGATAAYQVEGATKED |
| | | termitidis | | GKGRVLWDDFLEKQGRFSPDPAADFYH |
| | | | | RYDEDLALAEAYGHQVIRLSIAWSRIFP |
| | | | | DGAGAVEPRGVAFYHRLFAACAKHHLI |
| | | | | PFVTLHHFDTPERLHAIGDWLSQEMLED |
| | | | | FVEYARFCFEEFPEIKHWITINEPTSMAV |
| | | | | QQYTSGTFPPAETGHFDKTFQAEHNQIV |
| | | | | AHARIVNLYKSMGLDGEIGIVHALQTPY |
| | | | | PYSDSSEDQHAADLQDALENRLYLDGT |
| | | | | LAGDYAPKTLALIKEILAANQQPMFKYT |
| | | | | DEEMAAIKKAAHQLDFVGVNNYFSKWL |
| | | | | RAYHGKSETIHNGDGSKGSSVARLHGIG |
| | | | | EEKKPAGIETTDWDWSIYPRGMYDMLM |
| | | | | RIHQDYPLVPAIYVTENGIGLKESLPAEV |
| | | | | TPNTVIADPKRIDYLKKYLSAVADAIQA |
| | | | | GANVKGYFVWSLQDQFSWTNGYSKRY |
| | | | | GLFFVDFPTQKRYVKQSAEWLKQVSQT |
| | | | | HVIPE |
| BthuBgl | | | 43 | MSKVIFPKGFLWGGAIAANQVEGAYVE |
| | | | | DGKGLTTVDLLPTGENRWDIMKGNIHSF |
| | | | | TPVEGEFYPSHEAIDFYHRYKEDIALFAE |
| | | | | MGFKALRVSIAWTRIFPNGDDEKPNEAG |
| | | | | LQFYDNLFDELLKHDIEPVVTMAHFDVP |
| | | | | IHLVEKYGSWRSRKLVDFFETYAKTIFN |
| | | | | RYKDKVKYWMTFNEINMLLHLPFMGA |
| | | | | GLAFKEGDNKKQIQYQAAHHQLVASAL |
| | | | | AVKACHEIIPDAKIGCMLAAGATYPYTC |
| | | | | NPDDIQRAMEQDRESFFFIDVQARGAYP |
| | | | | GYAKRFFTDNNVTIEMEKEDEAILKEHT |
| | | | | VDYIGFSYYASRATSTDPEVLKSITSGNV |
| | | | | FGSVENPYLEKSEWGWTIDPKGFRITAN |
| | | | | QLYDRYQKPLFVVENGLGAIDQLNDED |
| | | | | EVNDAYRIDYLEKHMIEMSEAIQDGVDII |
| | | | | GYTSWGPIDLVSASTGEMKKRYGYIYV |
| | | | | DKDNEGKGSLKRSKKDSFNWYKEVIAT |
| | | | | NGGSLES |
| BamyBgl | | | 44 | MKRFPDGFLWGGATAANQIEGAYKEGG |
| | | | | KGLSTADVSPDGIMSPFHETDDALNLYH |
| | | | | DAIDFYHRYQEDIALFAEMGFKAFRTSIA |
| | | | | WTRIFPNGDETEPNEEGLQFYDRLFDEL |
| | | | | RKHQIEPVVTISHYEMPLGLVKNYGGW |
| | | | | RNRRTVDFYERYARTVFTRYKDKVKY |
| | | | | WMTFNEINVVLHAPFTGGGLIFREGENK |
| | | | | QNTMYQAAHHQFVASALAVKAGHEIIP |
| | | | | DSQIGCMIAATTTYPMTPKPEDVYAALQ |
| | | | | KERSTLFFSDVQARGSYPGYMKRFFKEN |
| | | | | GITIEMKEGDEALLKEHTVDYIGFSYYM |
| | | | | SMTASTAPEDLAQSKGNLLGGVKNPYL |
| | | | | KSSEWGWQIDPKGLRITLNTLYDRYQKP |
| | | | | LFIVENGLGAVDQPEEDGSIQDDYRINYL |
| | | | | RDHLIEAREAIEDGVDLIGYTSWGPIDLV |
| | | | | SASTAEMKKRYGYIYVDRGNDGKGTFE |
| | | | | RKKKKSFYWYKDVIATNGESL |
| LlacBgl | Q9CFLO | Lactococcuss | 45 | MTFKTDFLWGGATAANQLEGAYDIDGK |
| | | lactis | | GLSVADAMPGGKERLAILASPEEDWTID |
| | | subsp. | | TEHFTYPNHDGIDHYHHFKEDIALFAEM |
| | | Lactis | | GFKAYRFSVAWSRIFPKGDETTPNEKGL |
| | | | | LFYDQLIDECLKYRIEPVITISHYEMPLNL |
| | | | | AKSYGGWKNRELIEFYVRFAKVLLERY |
| | | | | QDKVKYWMTFNEINSATFFSGLSQGLVP |
| | | | | SNGGDDKTNVFKAWHNQFVASAQAVK |
| | | | | FGHDLNKNLKLGCMSIYSTTYSEDANPV |
| | | | | NQLATQESIQEFNYFCNDVQVRGAYPAF |
| | | | | TNRLHRKHGVNSEVLEISEEDLKIIAEGT |
| | | | | VDYIGESYYMSTVESKTGEGVQASGNM |
| | | | | VLGGVKNPFLKESEWGWAIDPDGLRYA |
| | | | | LNDLYGRYQIPLFIVENGLGAIDKVEED |
| | | | | GTIQDDYRIDYLKKHIQSMSEAVEDGVE |
| | | | | LMGYTPWGCIDLVSASTGEMSKRYGFIY |
| | | | | VDLDDSGNGTNKRFKKKSFDWYKQVID |
| | | | | SNGTNL |
| Ent7Bgl | | | 46 | MSSREKKQLSSMPNDFLWGGAISATQV |
| | | | | EGAYNHDGKGLSNLDLALRCKKGEKRQ |
| | | | | ITQQVDVNQYYPSHRAIGFYESYQKDIQ |
| | | | | LFADMGFKSLRFSIQWSRIFPTGEEERPN |
| | | | | EAGLLFYEKILDELERHRIEPIITISHEDLP |
| | | | | ENLVTKYGSWKNRQVITFYLRFCEALFQ |
| | | | | RFSDRVRYWIPFNEINVITYMPYFSTGIH |
| | | | | TENYQEIFQMAHHQLVASAKAVQLGRK |
| | | | | YSSNYRFATMLMYGPTYPHNCHPESVF |
| | | | | QAMMDDEETYYFGDIQIRGYYSPWAKK |
| | | | | MLEQLGVQLAITEEDEQDLREGVVDFVS |
| | | | | ISYYMSWTTAPETAAGNMATGGKNPFL |
| | | | | EQSEWGWQVDPLGLRISLNRLYQRYEK |
| | | | | EIMIVENGLGAVDHCSENGEIYDDYRID |
| | | | | YLQQHLLAVKQAIVLDGVPVIGFTVWS |
| | | | | AIDSISASTGEIGKRYGLIYVDLDDEGQG |
| | | | | TLARKKKASFYWYQKIIESNGAEL |
| GkauBgl-2 | Q5KXG4 | Geobacillus | 47 | MSQQRKSIIPDDFLWGGAVTSFQTEGAW |
| | | kaustophilus | | NEGGKGLSIVDARPIPKGHSDWKVAVDF |
| | | | | YHRYKEDIALFKELGFTAYRTSIAWTRIF |
| | | | | PDGEGEPNEAGLAFYDAVFDELRANGIE |
| | | | | PVITLYHFDLPLALAKKYNGFASRKVVD |
| | | | | LFERYARTVFERYRGKVNYWLTFNEQN |
| | | | | LVLEQPHLWGAICPEDEDPEAFAYRVCH |
| | | | | NVFIAHAKAVKALREIAPEAKIGGMVTY |
| | | | | LTTYPATCRPEDALANVQAKELFIDFFFD |
| | | | | VFARGAYPRYVTNQLEKKGICLPLEAGD |
| | | | | EELLRSQTVDFLSFSYYQSQIVRHQEQDE |
| | | | | RIIKGLEPNPYLPKTKWGWAIDPIGLRIA |
| | | | | LKDVYARYEMPIFITENGIGLEEELNENG |
| | | | | TVDDDERIDYLRRHIEQMKMAMEEGVE |
| | | | | VIGYLMWGATDLLSSQGEMRKRYGVIF |
| | | | | VNRDDENLRDLKRYKKKSFYWFORVIR |
| | | | | TNGEEL |
| GeoYB | | | 48 | MKYTQLKPFPTGFLWGGSTSAYQVEGA |
| | | | | WNEDGKGPSVIDMAKHPEGTTDFKVAS |
| | | | | DHYHRYQEDIALLAEMGFKAYRFSIAW |
| | | | | TRIYPNGEGEVNPKGLEFYNNLINEIVRH |
| | | | | GIEPIVTIYHFDLPYALQTKGGWSNRATI |
| | | | | DAFVNYCRTLFEHFGDRVKYWLTINEQ |
| | | | | NMMILHGEAIGIVDPDSENPKKELYQQN |
| | | | | HHMFVAQAKAMALCHEMLPDAKIGPAP |
| | | | | NIATIYPASSKPEDVLAANTYSAIRNWLY |
| | | | | LDMAVYGRYNPTAWAYLEEKGYTPTIA |
| | | | | DGDMDILQNAKPDFIAFNYYTSQTVAAS |
| | | | | VGNESDIGHTGDQHITIGEPGVYKGASN |
| | | | | PNLPKNDFGWEIDPIGFRTTLREIYERYR |
| | | | | LPLIVTENGLGAYDRLEEGDIVNDTYRID |
| | | | | FLRNHIEQMRLAITDGVDVFGYCPWSAI |
| | | | | DLVSTHQGISKRYGFIYVNRDEFDLKDL |
| | | | | RRIRKQSFYWYQRVISSNGEQLD |
| GkauBgl-3 | Q5KUY7 | Geobacillus | 49 | MEHRHLKPFPPGFLWGAASAAYQVEGA |
| | | kaustophilus | | WNEDGKGLSVWDVFAKQPGRTFKGTN |
| | | | | GDVAVDHYHRYKEDVALMAEMGLKA |
| | | | | YRFSVSWSRVFPDGNGAVNEKGLDFYD |
| | | | | RLIEELRTHGIEPIVTLYHWDVPQALMD |
| | | | | AYGAWESRRIIDDFDRYAVTLFQRFGDR |
| | | | | VKYWVTLNEQNIFISLGYRLGLHPPGVK |
| | | | | DMKRMYEANHIANLANAKVIQSFRHYV |
| | | | | PDGKIGPSFAYSPMYPYDSRPENVLAFE |
| | | | | NAEEFQNHWWMDVYAWGMYPQAAW |
| | | | | NYLESQGLEPTVAPGDWELLQEAKPDF |
| | | | | MGVNYYQTTTVEHNPPDGVSEGVMNTT |
| | | | | GKKGTSTSSGIPGLFKTVRNPYVDTTNW |
| | | | | DWAIDPVGLRIGLRRIANRYRLPILITEN |
| | | | | GLGEFDTLEPDDIVNDDYRIDYLRRHIQE |
| | | | | IQRAITDGVDVLGYCVWSFTDLLSWLN |
| | | | | GYQKRYGFVYVNRDDESEKDLRRIKKK |
| | | | | SFYWYQRVIATNGAEL |
| PchrBgl | Q25BW5 | Phanerodontia | 50 | MSAAKLPKSFVWGYATAAYQIEGSPDK |
| | | chrysosporium | | DGREPSIWDTFCKAPGKIADGSSGDVAT |
| | | | | DSYNRWREDVQLLKSYGVKAYRFSLSW |
| | | | | SRIIPKGGRSDPVNGAGIKHYRTLIEELV |
| | | | | KEGITPFVTLYHWDLPQALDDRYGGWL |
| | | | | NKEEAIQDFTNYAKLCFESFGDLVQNWI |
| | | | | TFNEPWVISVMGYGNGIFAPGHVSNTEP |
| | | | | WIVSHHIILAHAHAVKLYRDEFKEKQGG |
| | | | | QIGITLDSHWLIPYDDTDASKEATLRAM |
| | | | | EFKLGRFANPIYKGEYPPRIKKILGDRLP |
| | | | | EFTPEEIELVKGSSDFFGLNTYTTHLVQD |
| | | | | GGSDELAGFVKTGHTRADGTQLGTQSD |
| | | | | MGWLQTYGPGFRWLLNYLWKAYDKPV |
| | | | | YVTENGFPVKGENDLPVEQAVDDTDRQ |
| | | | | AYYRDYTEALLQAVTEDGADVRGYFG |
| | | | | WSLLDNFEWAEGYKVRFGVTHVDYET |
| | | | | QKRTPKKSAEFLSRWFKEHIEE |
| SdegBgl-1 | Q21EM1 | Saccharophagus | 51 | MKTFNPDFVWGAASSAYQVEGATTTDG |
| | | degradans | | RGPSIWDAFSSIPGKTYHNQNADIACDH |
| | | | | YNRWQEDVAIMKEMGLKAYRFSISWSR |
| | | | | IFPTGRGEVNEKGVAFYNNLIDELIKNDI |
| | | | | TPWVTLFHWDFPLALQMEMDGLLNPAI |
| | | | | ADEFANYAKLCFARFGDRVTHWITLNEP |
| | | | | WCSAMLGHGMGSKAPGRVSKDEPYIAA |
| | | | | HNLLRAHGKMVDIYRREFQPTQKGMIGI |
| | | | | ANNCDWREPKTDSELDKKAAERALEFF |
| | | | | VSWFADPIYLGDYPASMRERLGERLPTF |
| | | | | SDEDIALIKNSSDFFGLNHYTTMLAEQT |
| | | | | HEGDVVEDTIRGNGGISEDQMVTLSKDP |
| | | | | SWEQTDMEWSIVPWGCKKLLIWLSERY |
| | | | | NYPDIYITENGCALPDEDDVNIAINDTRR |
| | | | | VDFYRGYIDACHQAIEAGVKLKGYFAW |
| | | | | TLMDNYEWEEGYTKRFGLNHVDETTGK |
| | | | | RTPKQSAIWYSTLIKDGGF |
| HsapCyBgl | Q9H227 | Homo | 52 | MAFPAGFGWAAATAAYQVEGGWDAD |
| | | sapiens | | GKGPCVWDTFTHQGGERVFKNQTGDV |
| | | | | ACGSYTLWEEDLKCIKQLGLTHYRFSLS |
| | | | | WSRLLPDGTTGFINQKGIDYYNKIIDDLL |
| | | | | KNGVTPIVTLYHFDLPQTLEDQGGWLSE |
| | | | | AIIESFDKYAQFCFSTFGDRVKQWITINE |
| | | | | ANVLSVMSYDLGMFPPGIPHFGTGGYQ |
| | | | | AAHNLIKAHARSWHSYDSLERKKQKGM |
| | | | | VSLSLFAVWLEPADPNSVSDQEAAKRAI |
| | | | | TFHLDLFAKPIFIDGDYPEVVKSQIASMS |
| | | | | QKQGYPSSRLPEFTEEEKKMIKGTADFF |
| | | | | AVQYYTTRLIKYQENKKGELGILQDAEI |
| | | | | EFFPDPSWKNVDWIYVVPWGVCKLLKY |
| | | | | IKDTYNNPVIYITENGFPQSDPAPLDDTQ |
| | | | | RWEYFRQTFQELFKAIQLDKVNLQVYC |
| | | | | AWSLLDNFEWNQGYSSRFGLFHVDFED |
| | | | | PARPRVPYTSAKEYAKIIRNNGLEAHL |
| RratCyBgl | | | 53 | MTVYKGGWDADGRGPCVWDTFTHQG |
| | | | | GERVFENQTGDVACGSYTLWEEDLKCI |
| | | | | KQLGLTHYRESLSWSRLLPDGTTGFINQ |
| | | | | KGIDYYNKIIDDLLRNGVTPIVAIYHFDL |
| | | | | PQALEDLGGWLSEAIVEAFDKYAQFCFS |
| | | | | TFGDRVKQWLTINEPNILALLAYDMGIF |
| | | | | APGVPHIGIGGYQAAHNLIKAHARSWHS |
| | | | | YDSLFREEQKGFVSLSLFFCWLEPADPN |
| | | | | SAIDQEATKRAINFHLDFFAKPIFIDGDYP |
| | | | | DVVKSQVASMSKKQGYPSSRLPEFTEEE |
| | | | | KKMIKGTADFFAVQYYTTRLVRHQDNK |
| | | | | KRELGFLQDVEIEFFPNPFWKNVGWIYV |
| | | | | VPWGIRKLLKYIKDTYNNPVIYITENGFP |
| | | | | QCDPPSLDDTQRWEYFRQTFQELFKAIH |
| | | | | VDDVNLQLYCAWSLLDNFEWNNGYSR |
| | | | | RFGLFHVDFEDPARPRTPYTSAKEYAKV |
| | | | | IRNNGLAGAM |
| CcanCyBgl | A0A8B7TQ98 | Castor | 54 | MAFPVGFGWGAATAAYQVEGGWDAD |
| | | canadensis | | GRGPCVWDTFTHQGGDRVFKNQTGDV |
| | | | | ACGSYTLWEEDLKCIKQLGLTHYRFSLS |
| | | | | WSRLLPDGTTGFINQKGIDYYNKIIDDLL |
| | | | | ANGVKPIVAIYHFDLPQALEDQGGWLSE |
| | | | | AIIEVEDKYSQFCESTFGDRVKQWITINE |
| | | | | PNTLATMAYDFGIFAPGVPHIGTGGYQA |
| | | | | AHNMIKAHAKSWHSYDSLFRKEQKGM |
| | | | | VSLSLFVCWLEPADPNSKPDQEAAKRAI |
| | | | | NFQLDFFAKPIFIDGDYPELVKSQIAYMS |
| | | | | KKQGYPSSRLPEFTEEEKKMIKGTADFF |
| | | | | AVQYYTSRLVKHQESNKGELGFLQDVG |
| | | | | IEYFPDPSWKGVGWIYVVPWGIRKLLKY |
| | | | | IKDMYNSPVIYITENGFPQCDPPSLDDTQ |
| | | | | RWEYFRQTFQELFKAIHVDKVNLQLYC |
| | | | | AWSLLDNFEWNNGYSRRFGLFHVDFED |
| | | | | PARPRVPYRSAKEYAKIIKSNGLEGPL |
| CporCyBgl | P97265 | Cavia | 55 | MAFPADLVGGLPTAAYQVEGGWDADG |
| | | porcellus | | RGPCVWDTFTHQGGERVFKNQTGDVAC |
| | | | | GSYTLWEEDLKCIKQLGLTHYRFSISWS |
| | | | | RLLPDGTTGFINQKGVDYYNKIIDDLLT |
| | | | | NGVTPVVTLYHFDLPQALEDQGGWLSE |
| | | | | ALIEVFDKYAQFCFSTFGNRVRQWITINE: |
| | | | | PNVLCAMGYDLGFFAPGVSQIGTGGYQ |
| | | | | AAHNMIKAHARAWHSYDSLFREKQKG |
| | | | | MVSLSLFCIWPQPENPNSVLDQKAAERA |
| | | | | INFQFDFFAKPIFIDGDYPELVKSQIASMS |
| | | | | EKQGYPSSRLSKFTEEEKKMIKGTADFF |
| | | | | AVQYYTTRFIRHKENKEAELGILQDAEIE |
| | | | | LFSDPSWKGVGWVRVVPWGIRKLLNYI |
| | | | | KDTYNNPVIYITENGFPQDDPPSIDDTQR |
| | | | | WECFRQTFEELFKAIHVDKVNLQLYCA |
| | | | | WSLLDNFEWNDGYSKRFGLFHVDFEDP |
| | | | | AKPRVPYTSAKEYAKIIRNNGLERPQ · |
| OpriCyBgl | | | 56 | MAFPAGFGWGAGTAAYQIEGGWDADG |
| | | | | RGPCVWDTFTHQGGDRIFKNQTGDVAC |
| | | | | NSYTLWEEDLKCIKQLGLTHYRFSLSWS |
| | | | | RLLPDGTTGFINQKGVDYYNKIIDDLLK |
| | | | | NKIIPIVTLFHFDLPQALEDRGGWLSEATI |
| | | | | DIFDQYACFCFRTFGDRVKHWITINEAN |
| | | | | GFAILTYDLGFFAPGVPHIGTGGYQAAH |
| | | | | NLIKAHARAWHSYNSLFRKEQKGLVSLS |
| | | | | FFSVWLEPADPNSASDKKASERALAFEL |
| | | | | GTFAKPIFIDGDYPEVVKSQVASMSQRQ |
| | | | | GYPSSRLPEFTEEEKKMIKGTADFFAIQY |
| | | | | YTTRLIKHKENKKGELGFLQDVEIDCST |
| | | | | DPSWKGENWVCVVPWGLRKLLKHVKD |
| | | | | TYNNPVIYITENGFPQRDPPSLDDTQRW |
| | | | | ECFRQTFQELSKAIQVDKVNVQVYCAW |
| | | | | SLLDNFEWNDGYNTRFGLYHVDFEDPA |
| | | | | RPRVPYTSAKEYAKVIRNNGLEEKP |
| CasinPRI | A0A2R6RAC3 | Actinidia | 57 | MAQISSFNRTSFPDGFVFGIASSAYQFEG |
| | | chinensis | | AAKEGGKGPNIWDTFTHEFPGKISNGST |
| | | var. | | GDVADDFYHRYKEDVKVLKFIGLDGFR |
| | | chinensis | | MSISWARVLPRGKLSGGVNKEGIAFYNN |
| | | | | VINDLLSKGIQPFITIFHWDLPQALEDEY |
| | | | | GGFLSPHIVNDFRDFAELCFKEFGDRVK |
| | | | | HRITMNEPWSYSYGGYDAGLLAPGRCS |
| | | | | AFMAFCPKGNSGTEPYIVTHNLLLSHAA |
| | | | | AVKLYKEKYQAYQKGQIGITLVTYWMI |
| | | | | PYSNSKADKDAAQRALDFMLGWFIEPLS |
| | | | | FGEYPKSMRRLVGKRLPRFTKEQAMLV |
| | | | | KGSFDFLGLNYYIANYVLNVPTSNSVNL |
| | | | | SYTTDSLSNQTAFRNGVAIGRPTGVPAFF |
| | | | | MYPKGLKDLLVYTKEKYNDPVIYITENG |
| | | | | MGDNNNVTTEDGIKDPQRVYFYNQHLL. |
| | | | | SLKNAIAAGVKVKGYFTWALLDNFEWL |
| | | | | SGYTQRFGIVYVDFKDGLKRYPKDSAL |
| | | | | WFKK |
| CcelBgl | B815U2 | Ruminiclostridium | 58 | MAFKEGFVWGTATASYQIEGAVNEGGR |
| | | cellulolyticum | | GESVWDEFCRMKGKIDDDDNGDSACDS |
| | | | | YHRYSEDIQLMKEIGIKAYRFSISWTRILP |
| | | | | DGIGEINMEGVNYYNNLINGLLENGIEP |
| | | | | YVTLFHWDYPMELQYKGGWLNPESPL |
| | | | | WFENYAAICSRLFSDRVKYWITSNESQC |
| | | | | YIGFGYGTGWHAPGFKLPVNQVVRAW |
| | | | | HHNLKGLGLAAKAIRENAKGEVKVGLV |
| | | | | ACGEVGIPASDSEADMQAARNVLFDRE |
| | | | | HSEDSIDFGYGDLFEPALKGEYPKSLIPY |
| | | | | LPKGWQEDMKDICVPLDFLGVNAYIGSI |
| | | | | VEACENKKYRHLKLPVGIGKTSMEWPF |
| | | | | KPETLYWVTRFISERYKLPVYITENGMA |
| | | | | NNDWISTDGKINDTQREDYLNQYLSALS |
| | | | | KSIDDGADVRGYFYWSLLDNFEWAYGY |
| | | | | AKRFGLVYVDYSNFSRTLKQSALRYKKI |
| | | | | IELNGEVLK |
| TnonBgl | | | 59 | MTENAEKFLWGVATSAYQIEGATQEDG |
| | | | | RGPSIWDTFARRPGAIRDGSTGEPACDH |
| | | | | YHRYEEDIALMQSLGVGVYRFSVAWPRI |
| | | | | LPEGRGRINPKGLAFYDRLVDRLLAAGI |
| | | | | TPFLTLYHWDLPQALEDRGGWRSRETA |
| | | | | FAFAEYAEAVARALADRVPFFATLNEP |
| | | | | WCSAFLGHWTGEHAPGLRNLEAALRAA |
| | | | | HHLLLGHGLAVEALRAAGARRVGIVLN |
| | | | | FAPAYGEDPEAVDVADRYHNRYFLDPIL |
| | | | | GRGYPESPFQDPPPAPILSRDLEAIARPLD |
| | | | | FLGVNYYAPVRVAPGTGPLPVRYLPPEG |
| | | | | PVTAMGWEVYPEGLYHLLKRLGREVP |
| | | | | WPLYITENGAAYPDLWTGEAVVEDPER |
| | | | | VAYLEAHVEAALRAREEGVDLRGYFV |
| | | | | WSLMDNFEWAFGYTRRFGLYYVDFPSQ |
| | | | | RRIPKRSALWYRERIARAQTGGSAH |
| TourBgl | DIA786 | Thermomonospora | 60 | MAFTADFRWGVATAAYQIEGAVTEDGR |
| | | curvata | | GASVWDTFCHESGRIAGGHTGDVACDH |
| | | | | YHRWPEDLALMADLGVDAYRFSIAWPR |
| | | | | VQPGGRGPANPKGLDFYERLVDGLLER |
| | | | | GITPFVTLFHWDLPQALEDAGGWLSRDT |
| | | | | AHRFADYAALVAGRLGDRVEHWITLNE. |
| | | | | PVVVTAYGYAFGVYAPGRTLLLDALPT |
| | | | | AHHQLLGHGLAVAALREHGRRQKIGLA |
| | | | | NHYSPAWAQDESSPADRRAAQIFDLFM |
| | | | | NRLFTDPVLHGTLPDLSALGGPDPASYV |
| | | | | RDGDLAAIAAPIDFLGVNYYQPTRLQAP |
| | | | | PAGGPLPFEIVPITGHPVTGMGWPVVPD |
| | | | | ALLSLLRDLRRTHGDALPPILITENGCSY |
| | | | | DDAPGPDGTVDDPERIDFLRAHLQAVET |
| | | | | ALAEGIDVRGYFVWSLMDNFEWSEGYG |
| | | | | PRFGLVHIDYDTQRRTPKTSFAWYRDHI |
| | | | | ARARRTS |
| TbisBgl | D6Y5B2 | Thermobispora | 61 | MTAAEQRPLAPGAFPEGFVWGTATSAY |
| | | bispora | | QIEGAVDADGRGPSIWDVFCRVPGAIAR |
| | | | | GESGDHACDHYHRWREDVALMSELGV |
| | | | | GAYRESVAWPRVLPEGAGRVEQRGLDF |
| | | | | YRRLVDELRARDIEPFVTLYHWDLPQAL |
| | | | | EDRGGWRVRDTAERFADYAEVVAGAL |
| | | | | GDRVRYWITLNEPYCSAIAGYAEGRHAP |
| | | | | GAREGHGALAAAHHLLLGHGLATERLR |
| | | | | GRPGLRVGITLNMSPAVPAGPAPEDAAA |
| | | | | ARRMDLLVNRQFTDPLLGRRYPEDMAE |
| | | | | TFGAITDFSFRREGDLEIIGAPLDFLGVN |
| | | | | YYYRIHAAAAPYEQPDPARRTAADIGAR |
| | | | | TVVPEGVRTSGLGWPVEPEGLHQTLTW |
| | | | | LARRYPGLPPIYITENGYGDDGTLQDDG |
| | | | | RIAYLRDHLAALADAIADGVDVRGWFC |
| | | | | WSLLDNFEWARGYAARFGLVHVDYAT |
| | | | | QARTPKASFHWLRAFLREHAPAGPDQR |
| | | | | SGSPSSTR |
| DdesBgl | CICXP6 | Deinococeus | 62 | MTLTRKDFPNGFIFGTATSSYQIEGAASE |
| | | deserti | | DGRGPSIWDTFCRQPGRIQDGTSGDVAC |
| | | | | DHYHLWPEDLDLLRELGVDAYRFSLAW |
| | | | | PRIQPSGSGAVNEKGLEFYDRLVDGLLE |
| | | | | RGIQPYATLYHWDLPQPLQDIGGWANR |
| | | | | EVAHHFADYAALVAGRLGDRVRSIATL |
| | | | | NEPWCSSFLSYDIGEHAPGLRDRRLALA |
| | | | | AAHHLLLGHGQAVQAMRALGKPAELG |
| | | | | LVLNLTPAYPASQSAEDARATQYADGY |
| | | | | ANRWFLDPVFRGAYPQDMWDAFGQDV |
| | | | | PDVQDGDLALIREPLDFLGVNYYTRSLV |
| | | | | SAQGPVRPQDAEYTHMHWEVYPQGLT |
| | | | | DLLLRLQREYPVPPMYITENGAAYPDER |
| | | | | GHADIVHDPERLAYYQRHLAAVIEATRQ |
| | | | | GADVRGYFAWSMLDNFEWAYGYSRRF |
| | | | | GLFYVDYQTQERTWKDSGRWFQGLMA |
| | | | | RTPVAAD |
| CflaBgl | D5ULE7 | Cellulomonas | 63 | MTSTTRPSGRAFPADFLWGSATASYQIE |
| | | flavigena | | GAVAEDGRAPSIWDTESHTPGKVLDGD |
| | | | | TGDVAVDHYHRVPQDVAIMQDLGLQA |
| | | | | YRFSISWSRVLPAGTGEVNQAGLDFYSD |
| | | | | LVDRLIAADIKPVVTLYHWDLPQTLEDA |
| | | | | GGWTNRATAEAFAAYARVVARALGDR |
| | | | | VHLWTTLNEPWCSAFLGYGSGVHAPGV |
| | | | | TDPAAALAAVHHLNLAHGLAATAIREE |
| | | | | LGAATPVSITLNLHVTRAASPAPADVEA |
| | | | | KRRIDTIANEVFLGPLLEGAYPERVFADT |
| | | | | AAISDWSFVQEGDLELIRVPIDLLGVNY |
| | | | | YSTGRVQHGTPPVGDGTPGPDGHRSSV |
| | | | | VSPWIGADNVEWLPQPGPHTAMGWNIE |
| | | | | PQGLVDLLLELHERYPELPLAITENGAAF |
| | | | | YDTVTDDGRVHDPDRVAYLHDHVDAV |
| | | | | GEARDKGVDVRGYFVWSLFDNFEWAY |
| | | | | GYDRRFGVVHVDYDTQVRTLKDSARW |
| | | | | YRELVRTGTIPTPESAASL |
| BbreBgl | P94248 | Bifidobacterium | 64 | MTMIFPKGFMFGTATAAYQIEGAVAEG |
| | | breve | | GRTPSIWDTFSHTGHTLNGDTGDVADDF |
| | | | | YHRWEDDLKLLRDLGVNAYRFSIGIPRV |
| | | | | IPTPDGKPNQEGLDFYSRIVDRLLEYGIA |
| | | | | PIVTLYHWDLPQYMASGDGREGGWLER |
| | | | | ETAYRIADYAGIVAKCLGDRVHTYTTLN |
| | | | | EPWCSAHLSYGGTEHAPGLGAGPLAFR |
| | | | | AAHHLNLAHGLMCEAVRAEAGAKPGLS |
| | | | | VTLNLQICRGDADAVHRVDLIGNRVFLD |
| | | | | PMLRGRYPDELFSITKGICDWGFVCDGD |
| | | | | LDLIHQPIDVLGLNYYSTNLVKMSDRPQ |
| | | | | FPQSTEASTAPGASDVDWLPTAGPHTEM |
| | | | | GWNIDPDALYETLVRLNDNYPGMPLVV |
| | | | | TENGMACPDKVEVGTDGVKMVHDNDR |
| | | | | IDYLRRHLEAVYRAIEEGTDVRGYFAWS |
| | | | | LMDNFEWAFGYSKRFGLTYVDYESQER |
| | | | | VKKDSFDWYRRFIADHSAR |
| TfusBgl | | | 65 | MTSQSTTPLGNLEETPKPDIRFPSDFVWG |
| | | | | VATASFQIEGSTTADGRGPSIWDTFCATP |
| | | | | GKVENGDTGDPACDHYNRYRDDVALM |
| | | | | RELGVGAYRFSIAWPRIQPEGKGTPVEA |
| | | | | GLDFYDRLVDCLLEAGIEPWPTLYHWD |
| | | | | LPQALEDAGGWPNRDTAKREADYAEIV |
| | | | | YRRLGDRITNWNTLNEPWCSAFLGYAS |
| | | | | GVHAPGROEPAAALAAAHHLMLGHGL |
| | | | | AAAVMRDLAGQAGRSVRIGVAHNQTT |
| | | | | VRPYTDSEADRDAARRIDALRNRIFTEPL |
| | | | | VKGRYPEDLIEDVAAVTDYSFVQDGDL |
| | | | | KTISANLDMMGVNFYNPSWVSGNRENG |
| | | | | GSDRLPDEGYSPSVGSEHVVEVDPGLPV |
| | | | | TAMGWPIDPTGLYDTLTRLANDYPGLPL |
| | | | | YITENGAAFEDKVVDGAVHDTERIAYLD |
| | | | | SHLRAAHAAIEAGVPLKGYFAWSFMDN |
| | | | | FEWALGYGKRFGIVHVDYESQTRTVKD |
| | | | | SGWWYSRVMRNGGIFGQE |
| TterBgl | DICGH4 | Thermobaculum | 66 | MSQPRTDLAPGRFPADFTWGTATAAYQI |
| | | terrenum | | EGAVREDGRGVSIWDRFSHTPGKTHNG |
| | | | | DTGDVACDHYHRWQGDIELMRRLHVN |
| | | | | AYRFSIAWPRILPEGWGRVNPPGLDFYD |
| | | | | RLVDGLLAAGITPWVTLYHWDLPQALE |
| | | | | DRGGWPNPDTSKAFAEYADVVTRRLGD |
| | | | | RVKHWITLNEPWVVAFLGYFTGEHAPG: |
| | | | | RKEPESYLPVVHNLLLAHGLAVPVIREN |
| | | | | SRDSQVGITLNLTHAYPAGDSAEDEAAA |
| | | | | KRLDGFMNRWFLDPLFTGGYPRDMIDV |
| | | | | FGSWVPSFDESDLGVIGAPLDFLGVNYY |
| | | | | SPSFVQHSEGNPPLHVEQVRVDGEYTD |
| | | | | MGWLVYPQGLYDLLTRLHRDYSPAAIVI |
| | | | | TENGAAYPDEPPVEGRVHDPKRVEYYA |
| | | | | SHLDAAQRAIRDGVPLRGYFAWSLMDN |
| | | | | FEWAFGYSKRFGLYYVDYETLERTIKDS |
| | | | | GLWYSRVVAEGQLVPTESVA |
| SdegBgl-2 | Q21KX3 | Saccharophagus | 67 | MNRLTLPPSSRLRSKEFTFGVATSSYQIE |
| | | degradans | | GGIDSRLPCNWDTFCEQPNTIIDNINGAI: |
| | | | | ACDHINRWQDDIELIANLGVDAYRFSIA |
| | | | | WGRVINLDGSLNNEGVTFYKNILTKLRE |
| | | | | KNLKAYITLYHWDLPQHLEDAGGWLNR |
| | | | | DTAYKFRDYVNLITQALDDDVFCYTTL |
| | | | | NEPFCSAYLGYEIGVHAPGIKDLASGRK |
| | | | | AAHHLLLAHGLAMQVLRKNCPNSLSGI |
| | | | | VLNMSPCYAGSNAQADIDAAKRADDLL |
| | | | | FQWYAQPLLTGCYPDAINSLPDNAKPPI |
| | | | | CEGDMALISQPLDYLGLNYYTRAVFFAD |
| | | | | GNGGFTEQVPEGVELTDMGWEVYPQGL |
| | | | | TDLLIDLNQRYTLPPLLITENGAAMVDE |
| | | | | LVNGEVNDIARINYFQTHLQAVHNAIEQ |
| | | | | GVDVRGYFAWSLMDNFEWALGYSKRF |
| | | | | GITYVDYQTQKRTLKASGHAFAEFVSSR |
| | | | | S |
| VvulBgl | Q7MG41 | Vibrio | 68 | MNKYQLPQDSQLRQADFLFGVATSSYQI |
| | | vulnificus | | EGGAQLGGRTPSIWDTFCNQPGAVDNM |
| | | | | DNGDVACDHFHLWQQDIELIQGLGVDA |
| | | | | YRLSMAWPRILPKDGQVNQQGLEFYERI |
| | | | | IDECHARGLKVFVTLYHWDLPQYLEDK |
| | | | | GGWLNRETAYKFAEYAEVVSGYFGNKI |
| | | | | DSYATLNEPFCSAYLGYRWGIHAPGKK |
| | | | | GEREGFLSAHHLMLAHGLAMPIMRKNA |
| | | | | PQSMHGCVFNATPAYPYSEQDVAAAEY |
| | | | | SDAEGFHWFIDPVLKGEYPQSVLEHQAH |
| | | | | NMPMILDGDLDIIRGDLDFIGINFYTRCV |
| | | | | VREDANGELESMPQPDAEHTYIGWEIYP |
| | | | | QALTDLLLRLKQRYPNLPPVYITENGAA |
| | | | | GEDACINGEVNDEQRVRYFQSHLLALDE |
| | | | | AIRAGVNVQGYFAWSLMDNFEWAYGY |
| | | | | KQRFGIVHVDYATQKRTLKQSAIAYRNT |
| | | | | LLARAEEKQ |
| HoreBgl | B8CYA8 | Halothermothrix | 69 | MAKIIFPEDFIWGAATSSYQIEGAFNEDG |
| | | orenii | | KGESIWDRESHTPGKIENGDTGDIACDH |
| | | | | YHLYREDIELMKEIGIRSYRFSTSWPRILP |
| | | | | EGKGRVNQKGLDFYKRLVDNLLKANIR |
| | | | | PMITLYHWDLPQALQDKGGWTNRDTA |
| | | | | KYFAEYARLMFEEFNGLVDLWVTHNEP |
| | | | | WVVAFEGHAFGNHAPGTKDFKTALQV |
| | | | | AHHLLLSHGMAVDIFREEDLPGEIGITLN |
| | | | | LTPAYPAGDSEKDVKAASLLDDYINAW |
| | | | | FLSPVFKGSYPEELHHIYEQNLGAFTTQP |
| | | | | GDMDIISRDIDFLGINYYSRMVVRHKPG |
| | | | | DNLFNAEVVKMEDRPSTEMGWEIYPQG |
| | | | | LYDILVRVNKEYTDKPLYITENGAAFDD |
| | | | | KLTEEGKIHDEKRINYLGDHFKQAYKAL |
| | | | | KDGVPLRGYYVWSLMDNFEWAYGYSK |
| | | | | RFGLIYVDYENGNRRFLKDSALWYREVI |
| | | | | EKGQVEAN |
| CtheBgl | P26208 | Acetivibrio | 70 | MSKITFPKDFIWGSATAAYQIEGAYNED |
| | | thermocelluis | | GKGESIWDRFSHTPGNIADGHTGDVACD |
| | | | | HYHRYEEDIKIMKEIGIKSYRFSISWPRIF |
| | | | | PEGTGKLNQKGLDFYKRLTNLLLENGIM |
| | | | | PAITLYHWDLPQKLQDKGGWKNRDTTD |
| | | | | YFTEYSEVIFKNLGDIVPIWFTHNEPGVV |
| | | | | SLLGHFLGIHAPGIKDLRTSLEVSHNLLL |
| | | | | SHGKAVKLFREMNIDAQIGIALNLSYHY |
| | | | | PASEKAEDIEAAELSFSLAGRWYLDPVL |
| | | | | KGRYPENALKLYKKKGIELSFPEDDLKLI |
| | | | | SQPIDFIAFNNYSSEFIKYDPSSESGFSPA |
| | | | | NSILEKFEKTDMGWIIYPEGLYDLLMLL |
| | | | | DRDYGKPNIVISENGAAFKDEIGSNGKIE |
| | | | | DTKRIQYLKDYLTQAHRAIQDGVNLKA |
| | | | | YYLWSLLDNFEWAYGYNKRFGIVHVNF |
| | | | | DTLERKIKDSGYWYKEVIKNNGF |
| BacGBgl | AOALIOZQ | Cohnella | 71 | MASIQFPKDFVWGTATASYQIEGAYNED |
| | D8 9BACL | sp. OV330 | | GRGMSIWDTFSRTPGKVVNGDTGDIAC |
| | | | | DSYHRYEEDIALLKNLGVKAYRFSIAWP |
| | | | | RIYPDGDGELNQKGLDYYAKVIDGLLA |
| | | | | AGIEPCVTLYHWDLPQALQDKGGWDNR |
| | | | | DTIRAFVRYAETAFKAFGGKVKQWITFN |
| | | | | ETWCVSFLSNYIGAHAPGNTDLQLAVN |
| | | | | VAHNCMVAHGEAVKAFRALGISGEIGT |
| | | | | THNLYWFEPYTTKPEDVAAAHRNRAYN |
| | | | | NEWFMDPTFKGQYPQFMVDWFKGKGV |
| | | | | EVPIQPGDMETIAQPIDFIGVNFYSGGFG |
| | | | | RYKEGEGLEDCEEVQVGFDKTFMDWN |
| | | | | VYAEGLYKVLSWVHEEYGDVPIYITENG |
| | | | | ACYEDELTQEGRVHDAKRADYFKKHFI |
| | | | | QCHRLIESGVPLKGYFAWSLLDNFEWAE |
| | | | | GYVKRFGIVYTDYKTLKRYPKDSYRFIQ |
| | | | | SVIENDGFEA |
| BhalBgl | Q9KBK3 | Halalkali | 72 | MSIIQFPKEMKWGVATASYQIEGAINAG |
| | | bacterium | | GRGASIWDVFAKTPGKVKNGDNGDVA |
| | | halodurans | | CDSYHRYEEDIEIMKDLGVDMYRESVA |
| | | | | WPRIFPNGTGEVSREGLDYYHRLVDRLT |
| | | | | ENGIQPMCTLYHWDLPQALQEKGGWD |
| | | | | NRDTIDAFVRYAEVMFKEFGDKINHWIT |
| | | | | FNELWCVSFLSNYIGVHAPGNTDLQLAT |
| | | | | NVAHHLLVAHGKAVQSYRKMGLDGQI |
| | | | | GYAPNVEWNEPFSNQMEDAEACKRGN |
| | | | | GWFIEWFMDPVFKGAYPSFLVEWFEKK |
| | | | | GITVPIEAGDMETIQQPIDFLGINYYTGSV |
| | | | | ARYKENEGLFDLEKVDAGYEKTDIGWN. |
| | | | | IYPEGFYKVLYYITEQYGQIPIYITENGSC |
| | | | | YNDEPVNGQVKDEGRIRYLSQHLTALK |
| | | | | RSMESGVNIKGYMAWSLLDNFEWAEG |
| | | | | YSMRFGIVHVNYRTLERTKKDSFYWYK |
| | | | | QMIANOFFEL |

In some embodiments, the glycosidase can be a rutinosidase. In one embodiment, rutinosidase can include one or more enzymes from Table 3. In one embodiment, the compositions of the disclosure can include a rutinosidase having about 50%, 55%, 60%, 65%, 70%, 75%, 80%, 85%, 90%, 91%, 92%, 93%, 94%, 95%, 96%, 97%, 98%, 99%, or 100% to the sequences in Table 3. In one embodiment, the sequences in Table 3 can further be mutated to tune the enzymatic activity of the sequences. In some embodiments, the rutinosidase is AoryRut derived from UniProt ID: A0AIS9DRB1. In some embodiments, the rutinosidase is CtroEXG derived from UniProt ID: C5ME42. In some embodiments, the rutinosidase is CmalEXG derived from UniProt ID: M3IJY9. In some embodiments, the rutinosidase is AcreRut derived from UniProt ID: A0A286JZ59. In some embodiments, the rutinosidase is AniRut derived from UniProt ID: A0A6B9UJ04. In some embodiments, rutinosidases of the disclosure derived from UniProt sequences described herein does not include the native leader sequence or signal peptide sequence.

TABLE 3
Rutinosidase sequences

| Name: | Organism | SEQ ID | Sequence |
|---|---|---|---|
| AoryRut | Aspergillus | 73 | MAPHPRVQSPEYVNWTTFKANGVNLGGWLVQ |
| | oryzae | | ESTIDSQFWGTYSGGADDEWGLCEHLGSRCGPV |
| | | | LEHRYATYITERDIDKLASVGVGVLRIPTTYAA |
| | | | WIKLPGSQLYSGNQTAYLKQIADYAITKYGMHII |
| | | | VDVHSLPGGTNGLTIGEASGHWGWYYNETAFD |
| | | | YSMQVIDAVISFVQNSGSPQSYTIEPMNEPTDNP |
| | | | DMSVFGTPAALSDRGATWVLKYIRAVIDRVAS |
| | | | VNPNIPVMFQGSFKPEQYWSNQLPADANLVFD |
| | | | VHTYYFERNVTSETLPARLYADAQSKAGDGKFP |
| | | | VFTGEWAIQTLYQNSFALRERNVNAGLDAMYK |
| | | | YSQGSCYWTAKFSGNATVNGQGTQADYWNFE |
| | | | YFIDHGYIDLTRFHDTK |
| CtroEXG | Candida | 74 | MISNPSKSNGVKFKRGGNVAWDYENDIVRGVN |
| | tropicalis | | LGGWFVLEPYMNPSLFEPFKNGNDESGVPVDEY |
| | | | HWTQTLGKETASKILEDHWAKWITEWDFQQMS |
| | | | NLGLNLVRIPIGYWAFQLLDNDPYVQGQVAFLD |
| | | | EALEWARNHNIKVWIDLHGAPGSQNGFDNSGL |
| | | | RDSLEFQNGDNTQVTLNVLAEIFQKYGTSDYDD |
| | | | VVVGIELVNEPLGPSLDMDALKKFYMDGYSSLR |
| | | | NTEGSVTPLIIHDAFQVSGYWDNFLTVAGGQW |
| | | | NVVLDHHHYQVFSAGELSRDIDQHISVACNWG |
| | | | WSAKNEYHWTVTGEWSAALTDCAYWLNGVN |
| | | | RGARWEGAYDGSPYYGSCEPYLQFSSWTDEHK |
| | | | TNVRRYIEAQLDAFEETGGWIFWSWKTENAID |
| | | | WDFQKLTDNGIFPQPLDDRQFPNQCGFN |
| CmalEXG | Candida | 75 | MITNPQNNNNNNNVKFKRGGTVAWDYDNDTIR |
| | maltosa | | GVNLGGWFVLEPYMNPSLFQPFSSGNGDVGIPL |
| | | | DEYHETQTLGKDAASEILQKHWSTWITEDDFQQ |
| | | | MSSLGLNFARIPIGYWAFELLSNDPYVQGQVEY |
| | | | LDQALEWARNSNIKVWIDLHGAPGSQNGFDNS |
| | | | GLRDSLQFQNGDNTQATLNALAKIFQKYGGAN |
| | | | YSDVVIGIELLNEPLGPSLDMSALQQFFVEGYWS |
| | | | LRNTDGSVTPVIIHDAFQPFGYWDNFLTVANGE |
| | | | WNVVIDHHHYQVFSPGELSRDINQHISVACNWG |
| | | | WDAKKEYHWNIAGEWSAALTDCATWLNGVGR |
| | | | GARWEGAYDGSQYFGSCQPYLQFETWPEDYKT |
| | | | NVRKYVEAQLDAFEYTGGWVFWSWKTENAIE |
| | | | WDFQKLTANGIFPQPLTDRWYPNQCGFN |
| AcreRut | Acremonium | 76 | MAPQAAYLDWKAFRANGVNLGGWLHQEAVID |
| | sp. DSM | | PVWWSENGGDGIPDEWGLCAKLGRLCGPRLEQ |
| | 24697 | | RYASYITTQDIDEMAEAGINVLRIPTGYNAWVK |
| | | | VPGSQLYTGNQVRFLRSISDYAIRKYGMHIIVDI |
| | | | HSAPGGLNGMGLGGREGGYGWFQNETALDYSF |
| | | | RAVDAAIAFIQSSSHPESFTLEPLNEPVDNRNMA |
| | | | EFGTPAALTPEGVAWVLKYFRGVLSRVQKVDA |
| | | | RIPVMLQGSFKGEDFWSPYFAATDNIVFDVHHY |
| | | | YFAGRPTTSANLPEWICTDAKGAVGDGVFPVFT |
| | | | GEWSIQAATANTFASRALNLNTGLKVFGEYSRG |
| | | | SAYWTWKFSGNVPVEGEGVQGDYWSYEKFFE |
| | | | AGYINPSEGVSCQ |
| AniRut | Aspergillus | 77 | MAPLASPPNSSYIDWRTFKGNGVNLGGWLEQE |
| | niger | | STIDSLFWDKYSGGASDEWGLCEHLGSQCGPVL |
| | | | EHRYATLITKADIDKLASGGITVLRIPTTYAAWI |
| | | | DLPSSQLYSGNQTAYLKEIADYAIKTYNMHIIID |
| | | | THSLPGGVNGLTIGEATGHWYWFYNETHFNYS |
| | | | MQVIDQVINFIQTSGSPQSYTLEPINEPADNNTN |
| | | | MVVFGTPLALTDHGAAWVLKYIRAVVQRVESV |
| | | | NPNIPVMFQGSFKYPQYWEGDFPASTNLVFDTH |
| | | | HYYYEHMDSSSENLPEYILADAREKSGTGKFPV |
| | | | FVGEWAIQATYNNTLALRKRNVLAGLETWSSFS |
| | | | QGSSYWTAKFTGNTSVAGQGEQKDYWCYETFI |
| | | | DEGYFN |
| MC56 | — | 78 | MAPHPRVQSPEYVNWTTFKANGVNLGGWLVQ |
| | | | ESTIDSQFWGTYSGGADDEWGLCEHLGSRCGPV |
| | | | LEHRYATYITERDIDKLASVGVGVLRIPTTYAA |
| | | | WIKLPGSQLYSGNQTAYLKQIADYAITKYGMHII |
| | | | VDVHSLPGGVNGLTIGEASGHWGWYYNETAFD |
| | | | YSMQVIDAVISFVQNSGSPQSYTIEPINEPTDNPD |
| | | | MSVFGTPAALSDRGATWVLKYIRAVIDRVASV |
| | | | NPNIPVMFQGSFKPEQYWSNQLPADANLVFDV |
| | | | HTYYFERNVTSETLPARLYADAQSKAGDGKFPV |
| | | | FTGEWAIQTLYNNSFALRERNVNAGLDAMYKY |
| | | | SQGSCYWTAKFSGNATVNGQGTQADYWNFEY |
| | | | FIDHGYIDLTRFHDTK |

In one embodiment, the compositions of the disclosure can include a rutinosidase which has an amino acid sequence with a mutation at one or more positions. In one embodiment, the compositions of the disclosure can include a rutinosidase which has an amino acid sequence with a mutation at one or more positions of SEQ ID NO: 73. In some embodiments, the mutation can be a conservative or a non-conservative amino acid mutation. In one embodiment, the compositions of the disclosure can include a rutinosidase which has an amino acid sequence with a mutation at one or more of position 141, 190, 279, 307, 38, 39, 41, 87, 94, 145, 156, 168, 181, 183, 184, 214, 270, 276, 297, 324, 328, and/or 342 of SEQ ID NO: 73. In one embodiment, the composition can include a rutinosidase with a mutation at one or more positions such as, but not limited to position 141, 190, and/or 279 of SEQ ID NO: 73. In one embodiment, the composition can include a rutinosidase of SEQ ID NO: 78. In one embodiment, the composition can include a rutinosidase with a mutation at one or more positions such as, but not limited to position 141, 190, and/or 307 of SEQ ID NO: 73. In one embodiment the mutations include one or more of T141V M190I,Q307N, T297V,Q38D, F39W, G4IN, G87N, T94N, T141I, T145V, Y156F, V168M, S181Y, Q183W, S184F, T214A, N270R, L276K, R279H, M324W, S328T, and/or A342F relative to SEQ ID NO: 73. In one embodiment, the mutations can include one or more of T141V, M190I, and/or R279H relative to SEQ ID NO: 73. In one embodiment, the mutations can include one or more of T141V, M190I, and/or Q307N relative to SEQ ID NO: 73.

In one embodiment, the compositions of the disclosure can include glycosidases at a concentration of about 0.001 mg/mL, 0.002 mg/ml, 0.003 mg/ml, 0.004 mg/ml, 0.005 mg/mL, 0.006 mg/ml, 0.007 mg/ml, 0.008 mg/ml, 0.009 mg/ml, 0.01 mg/mL, 0.02 mg/mL, 0.03 mg/ml, 0.04 mg/mL, 0.05 mg/ml, 0.06 mg/ml, 0.07 mg/ml, 0.08 mg/mL, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 2 mg/ml, 2.5 mg/ml, 3 mg/ml, 3.5 mg/ml, 4 mg/ml, 4.5 mg/ml, 5 mg/ml, 5.5 mg/ml, 6 mg/ml, 6.5 mg/ml, 7 mg/ml, 7.5 mg/ml, 8 mg/ml, 8.5 mg/ml, 9 mg/ml, 9.5 mg/ml, 10 mg/ml, 10.5 mg/ml, 11 mg/ml, 11.5 mg/ml, 12 mg/ml, 12.5 mg/ml, 13 mg/ml, 13.5 mg/ml, 14 mg/ml, 14.5 mg/ml, 15 mg/ml, 15.5 mg/ml, 16 mg/ml, 16.5 mg/ml, 17 mg/ml, 17.5 mg/ml, 18 mg/ml, 18.5 mg/ml, 19 mg/ml, 19.5 mg/ml, 20 mg/ml, 25 mg/ml, 30 mg/ml, 40 mg/ml, 45 mg/ml, 50 mg/ml or more. In one embodiment, the compositions of the disclosure can include glycosidases at a concentration of about 0.1 to 1 mg/ml, 0.2 mg/ml to 1.2 mg/ml, 0.4 mg/ml to 5 mg/ml, 0.5 to 5 mg/ml, 1 to 10 mg/ml, 5 to 15 mg/ml, 10 to 20 mg/ml, 15 to 25 mg/ml, 20 to 30 mg/ml, 25 to 35 mg/ml, 30 to 40 mg/ml, 35 to 45 mg/ml, or 40 to 50 mg/ml, or more.

In one embodiment, the compositions of the disclosure can include glycosidases at a concentration of about 0.001 mg/ml to 50 mg/ml, for example, 0.001 mg/ml to 0.01 mg/ml, 0.005 mg/ml to 0.05 mg/ml, 0.01 mg/ml to 0.1 mg/ml, 0.05 mg/ml to 0.5 mg/ml, 0.1 to 1 mg/ml, 0.2 mg/ml to 1.2 mg/ml, 0.4 mg/ml to 5 mg/ml, 0.5 to 5 mg/ml, 1 to 10 mg/ml, 5 to 15 mg/ml, 10 to 20 mg/ml, 15 to 25 mg/ml, 20 to 30 mg/ml, 25 to 35 mg/ml, 30 to 40 mg/ml, 35 to 45 mg/ml, or 40 to 50 mg/ml, or more.

In one embodiment, the compositions of the disclosure can include glucoside and/or the gentiobioside hydrolyzing enzymes at a concentration of about 0.001 mg/mL, 0.002 mg/ml, 0.003 mg/ml, 0.004 mg/ml, 0.005 mg/mL, 0.006 mg/ml, 0.007 mg/ml, 0.008 mg/ml, 0.009 mg/ml, 0.01 mg/mL, 0.02 mg/mL, 0.03 mg/ml, 0.04 mg/mL, 0.05 mg/ml, 0.06 mg/ml, 0.07 mg/ml, 0.08 mg/mL, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 2 mg/ml, 2.5 mg/ml, 3 mg/ml, 3.5 mg/ml, 4 mg/ml, 4.5 mg/ml, 5 mg/ml, 5.5 mg/ml, 6 mg/ml, 6.5 mg/ml, 7 mg/ml, 7.5 mg/ml, 8 mg/ml, 8.5 mg/ml, 9 mg/ml, 9.5 mg/ml, 10 mg/ml, 10.5 mg/ml, 11 mg/ml, 11.5 mg/ml, 12 mg/ml, 12.5 mg/ml, 13 mg/ml, 13.5 mg/ml, 14 mg/ml, 14.5 mg/ml, 15 mg/ml, 15.5 mg/ml, 16 mg/ml, 16.5 mg/ml, 17 mg/ml, 17.5 mg/ml, 18 mg/ml, 18.5 mg/ml, 19 mg/ml, 19.5 mg/ml, 20 mg/ml, 25 mg/ml, 30 mg/ml, 40 mg/ml, 45 mg/ml, 50 mg/ml or more.

In one embodiment, the compositions of the disclosure can include glucoside and/or the gentiobioside hydrolyzing enzymes at a concentration of about 0.001 mg/ml to 50 mg/ml, for example, 0.001 mg/ml to 0.01 mg/ml, 0.005 mg/ml to 0.05 mg/ml, 0.01 mg/ml to 0.1 mg/ml, 0.05 mg/ml to 0.5 mg/ml, 0.1 to 1 mg/ml, 0.2 mg/ml to 1.2 mg/ml, 0.4 mg/ml to 5 mg/ml, 0.5 to 5 mg/ml, 1 to 10 mg/ml, 5 to 15 mg/ml, 10 to 20 mg/ml, 15 to 25 mg/ml, 20 to 30 mg/ml, 25 to 35 mg/ml, 30 to 40 mg/ml, 35 to 45 mg/ml, or 40 to 50 mg/ml, or more.

In one embodiment, the compositions of the disclosure can include rutinosidases at a concentration of about 0.001 mg/mL, 0.002 mg/ml, 0.003 mg/ml, 0.004 mg/ml, 0.005 mg/mL, 0.006 mg/ml, 0.007 mg/ml, 0.008 mg/ml, 0.009 mg/ml, 0.01 mg/mL, 0.02 mg/mL, 0.03 mg/ml, 0.04 mg/mL, .05 mg/ml, 0.06 mg/ml, 0.07 mg/ml, 0.08 mg/mL, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 2 mg/ml, 2.5 mg/ml, 3 mg/ml, 3.5 mg/ml, 4 mg/ml, 4.5 mg/ml, 5 mg/ml, 5.5 mg/ml, 6 mg/ml, 6.5 mg/ml, 7 mg/ml, 7.5 mg/ml, 8 mg/ml, 8.5 mg/ml, 9 mg/ml, 9.5 mg/ml, 10 mg/ml, 10.5 mg/ml, 11 mg/ml, 11.5 mg/ml, 12 mg/ml, 12.5 mg/ml, 13 mg/ml, 13.5 mg/ml, 14 mg/ml, 14.5 mg/ml, 15 mg/ml, 15.5 mg/ml, 16 mg/ml, 16.5 mg/ml, 17 mg/ml, 17.5 mg/ml, 18 mg/ml, 18.5 mg/ml, 19 mg/ml, 19.5 mg/ml, 20 mg/ml, 25 mg/ml, 30 mg/ml, 40 mg/ml, 45 mg/ml, 50 mg/ml or more.

In one embodiment, the compositions of the disclosure can rutinosidases at a concentration of about 0.001 mg/ml to 50 mg/ml, for example, 0.001 mg/ml to 0.01 mg/ml, 0.005 mg/ml to 0.05 mg/ml, 0.01 mg/ml to 0.1 mg/ml, 0.05 mg/ml to 0.5 mg/ml, 0.1 to 1 mg/ml, 0.2 mg/ml to 1.2 mg/ml, 0.4 mg/ml to 5 mg/ml, 0.5 to 5 mg/ml, 1 to 10 mg/ml, 5 to 15 mg/ml, 10 to 20 mg/ml, 15 to 25 mg/ml, 20 to 30 mg/ml, 25 to 35 mg/ml, 30 to 40 mg/ml, 35 to 45 mg/ml, or 40 to 50 mg/ml, or more.

In some embodiments, the compositions of the disclosure can include at least one glycosidase enzyme. As a non-limiting example, the glycosidases (also herein glycoside hydrolyzing enzyme) include an amino acid sequence of SEQ ID NO: 1-72. As an example, the glycosidases can include an amino acid sequence of SEQ ID NO: 4-13.

In one embodiment, the compositions of the disclosure can include at least one glucoside and/or gentiobioside hydrolyzing enzyme and at least one rutinosidase. In one embodiment, the compositions can include a glucoside and/or a gentiobioside hydrolyzing enzyme having an amino acid sequence with at least 85% sequence identity to SEQ ID NO: 1-72; and a rutinosidase having an amino acid sequence with at least 85% sequence identity to SEQ ID NO: 73-78. In one embodiment, the compositions of the disclosure can include the glucoside and/or a gentiobioside hydrolyzing enzyme of SEQ ID NO: 1 and the rutinosidase of SEQ ID NO: 78. In one embodiment, the compositions of the disclosure can include two, three, four, five, six, seven, eight, nine, ten or more glucoside and/or gentiobioside hydrolyzing enzyme. In one embodiment, the compositions of the disclosure can include two, three, four, five, six, seven, eight, nine, ten or more rutinosidase.

As a non-limiting example, the compositions of the disclosure can include the glucoside and/or the gentiobioside hydrolyzing enzyme CbBg1B-1 (MBR2796233.1; SEQ ID NO: 1) and the rutinosidase AoryRut (A0A1S9DRB1; SEQ ID NO: 73).

As a non-limiting example, the compositions of the disclosure can include the glucoside and/or a gentiobioside hydrolyzing enzyme CbBg1B-1 (MBR2796233.1; SEQ ID NO: 1) and the rutinosidase of SEQ ID NO: 78.

Also provided herein are polynucleotides encoding the glycosidases described herein.

In some embodiments, the present disclosure also provides cells engineered to express (i) a glucoside and/or a gentiobioside hydrolyzing enzyme with an amino acid sequence with at least 85% sequence identity to SEQ ID NO: 1-72; and/or (ii) a rutinosidase with an amino acid sequence with at least 85% sequence identity to SEQ ID NO: 73-78. The cell may be a eukaryotic cell or a prokaryotic cell. In some embodiments, the prokaryotic cell may be a bacterial cell e.g., E. coli. In some embodiments, the eukaryotic cells may be yeast cells, insect cells, and/or mammalian cells.

In some embodiments, the present disclosure provides methods for hydrolyzing volatile phenolics from phenolic glycosides. In some embodiments, the methods are for hydrolyzing volatile phenolics from phenolic glycosides in a fruit product or a fermented product thereof.

In some embodiments, the methods of the disclosure can involve incubating the fruit product or a fermented product thereof with the compositions described herein. In some embodiments, the fruit product or the fermented fruit product can be smoke-exposed.

In some embodiments, the methods of the disclosure are performed for about 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days or more,

In some embodiments, the methods of the disclosure are performed at room temperature. In some embodiments, the methods of the disclosure are performed at about 37 degrees C. In some embodiments, the methods of the disclosure are performed at about 30° C., 31° C., 32° C., 33° C., 34° C., 35° C., 36° C., 37° C., 38° C., 39° C., 40° C. or more. In one embodiment, the methods of the disclosure are performed at less than 37° C. In some embodiments, the methods of the disclosure are performed at greater than 37° C.

In some embodiments, the methods of the disclosure are performed at the pH of the fruit product or fermented product thereof. In some embodiments, the pH can be about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9,6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8. In some embodiments, the fruit product is derived from any fruit. In some embodiments, the fruit is a berry. Non-limiting examples of fruit include grapes, apples, blueberries, blackberries, raspberries, currants, strawberries, cherries, pears, peaches, nectarines, oranges, pineapples, mangoes, and/or passionfruit, In some embodiments, the fruit product may be derived from two or more different fruits. In some embodiments, the fruit is a grape. In some embodiments, the fruit product may be derived from one or more varieties of grapes. Non-limiting examples of grape varieties include, Cabernet Sauvignon, Alicante Henri Bouschet, Barbera, Bobal, Cabernet Franc, Carignan, Cinsaut, Malbec, Douce noir, Gamay, Grenache, Isabella, Merlot, Montepulciano, Mourvedre, Pinot noir, Sangiovese, Syrah, Tempranillo, Zinfandel, Aglianico, Blaufrankisch, Bordo, Carmenere, Castelão, Concord, Corvina Veronese, Criolla Grande, Croatina, Dolcetto, Dornfelder, Marufo, Mencia, Black Muscat, and/or Nebbiolo. In some embodiments, the fruit product can include fruit homogenate, a fruit juice, a fruit pulp, a fruit skin, a fruit peel, a fruit seed, a fruit concentrate, or combinations thereof.

In one embodiment, the methods of the disclosure can be applied to fermented fruit products, In one embodiment, the fruit product can be fermented after the methods of the disclosure are applied to the fruit product. In some embodiments, the fermented fruit product is a fermented beverage. In some embodiments, the fermented beverage is wine. In some embodiments, the wine can be table wine, dessert wine, fortified wine, sparkling wine, beer, spirits, cider, mead, liqueurs, sake, or brandy. In some embodiments, the table wine is red wine, white wine, a rose wine. In some embodiments, the red wine is Cabernet Sauvignon, Alicante Henri Bouschet, Barbera, Bobal, Cabernet Franc, Carignan, Cinsaut, Malbec, Douce noir, Gamay, Grenache, Isabella, Merlot, Montepulciano, Mourvedre, Pinot noir, Sangiovese, Syrah, Tempranillo, Zinfandel, Aglianico, Blaufrankisch, Bordo, Carmenere, Castelão, Concord, Corvina Veronese, Criolla Grande, Croatina, Dolcetto, Domfelder, Marufo, Mencia, Black Muscat, and/or Nebbiolo. In some embodiments, the white wine is Chardonnay, Sauvignon Blanc, Pinot Grigio, Moscato, Riesling, and/or Chenin Blanc. In some embodiments, the rosé wine is Provence Rosé Fresh, Grenache Rosé, Sangiovese Rosé, Syrah Rosé, Zinfandel Rosé, and/or Cabernet Sauvignon Rosé.

In some embodiments, the methods described herein may involve removing one or more volatile phenols from apparatus and containers involved in the wine making process or fruit fermentation process. Examples of apparatus and containers involved in the wine making process or fruit fermentation process include crushers/destemmers, fermentation vessels (stainless steel tanks, oak barrels, concrete tanks), presses (basket press, bladder press), pumps, airlocks and fermentation locks, hydrometers, refractometers, thermometers, primary fermenters (plastic food-grade buckets, glass carboys), secondary fermenters (glass carboys, stainless steel vessels), bottles, barrels, demijohns, kegs, fermentation buckets, and corks.

Any of the methods described herein may involve removing one or more volatile phenols from the fruit product or fermented fruit product. In some embodiments, removing or reducing the level of volatile phenols in the fruit product or fermented fruit product involves subjecting the fruit product or fermented fruit product to one or more additional processes, such as filtering (e.g., reverse osmosis), contacting the fruit product or fermented fruit product with a fining agent or other adsorbant/affinity agent (e.g., molecularly imprinted polymer), or modifying the volatile phenols (e.g., chemical modification such as methylation).

In some embodiments, the methods involve subjecting the fruit product or fermented fruit product to a filtration process. Filtration methods suitable for removal of volatile phenols from a fermented product are known in the art. In some embodiments, the filtration process is reverse osmosis, which involves passing the fruit product or fermented fruit product through a membrane (filter) having a molecular weight cut-off sufficient to remove volatile phenols from the fermented product.

In some embodiments, the methods involve contacting the fruit product or fermented fruit product with a fining or affinity agent. Examples of these agents for removal of smoke taint include activated carbon, molecularly imprinted polymers and cyclodextrin polymers.

In some embodiments, removing or reducing the level of volatile phenols in the fruit product or fermented fruit product involves subjecting the fruit product or fermented fruit product to an enzymatic process to modify the volatile phenol, for example contacting the fermented product with an enzyme capable of removing the undesired phenol or converting the undesired volatile phenol into a neutral or more desirable form.

The present disclosure also provides methods of quantifying the volatile phenolic and/or a phenolic glycoside in a fruit product or a fermented fruit product. The methods can include incubating the fruit product or fermented fruit product with the compositions of the disclosure. The levels are of the volatile phenolic and/or phenolic glycoside are then measured using mass spectrometry. In some embodiments, the mass spectrometry can be gas chromatography mass spectrometry or liquid chromatography mass spectrometry.

Presented below are examples discussing the utility of compounds of the invention contemplated for the discussed applications. The following examples are provided to further illustrate the embodiments of the present invention but are not intended to limit the scope of the invention, While they are typical of those that might be used, other procedures, methodologies, or techniques known to those skilled in the art may alternatively be used.

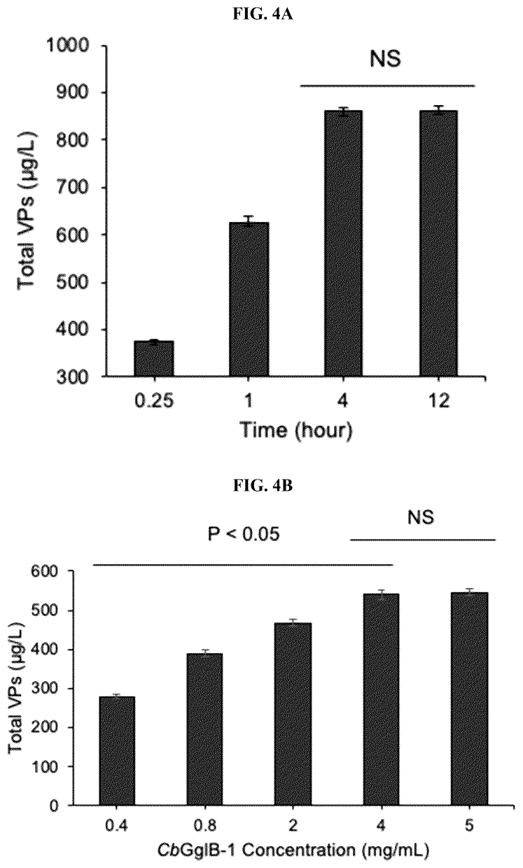

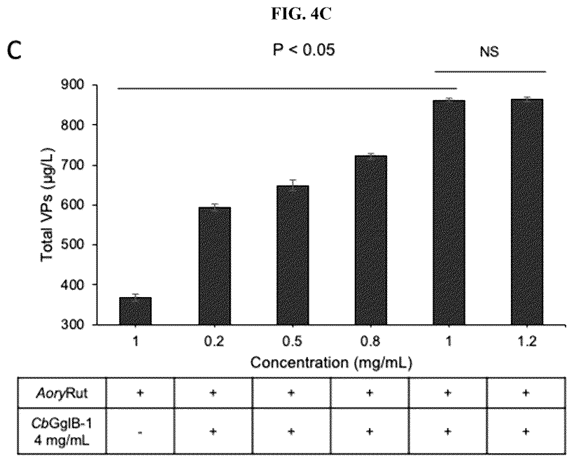

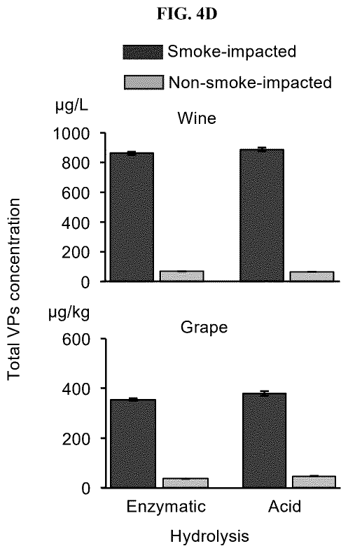

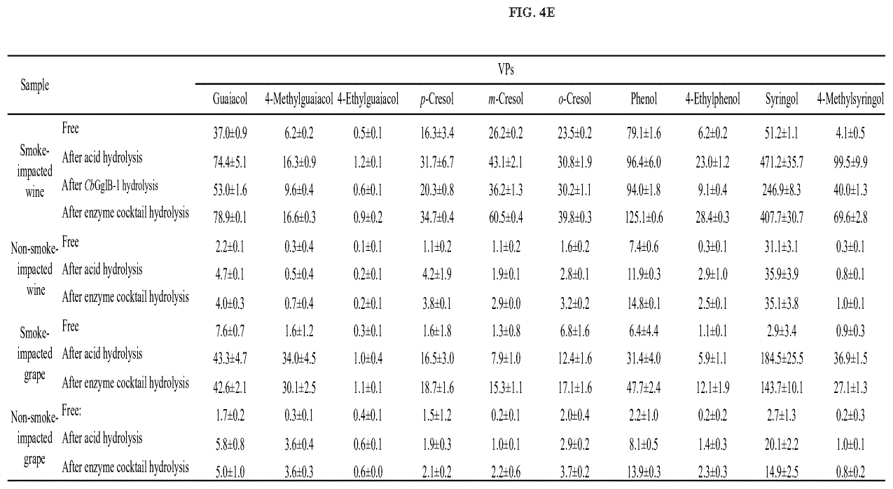

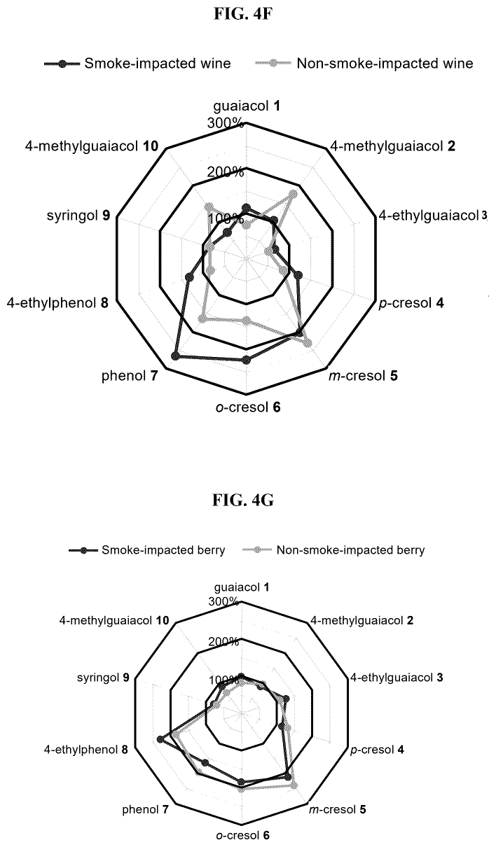

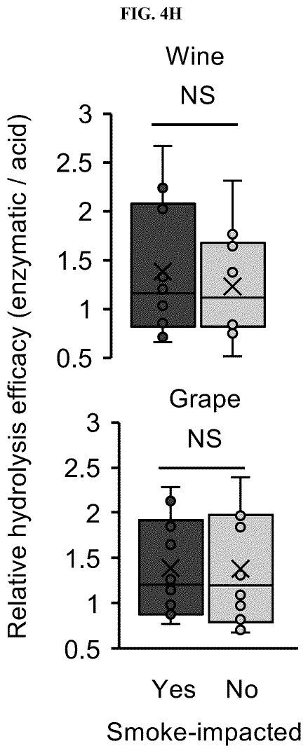

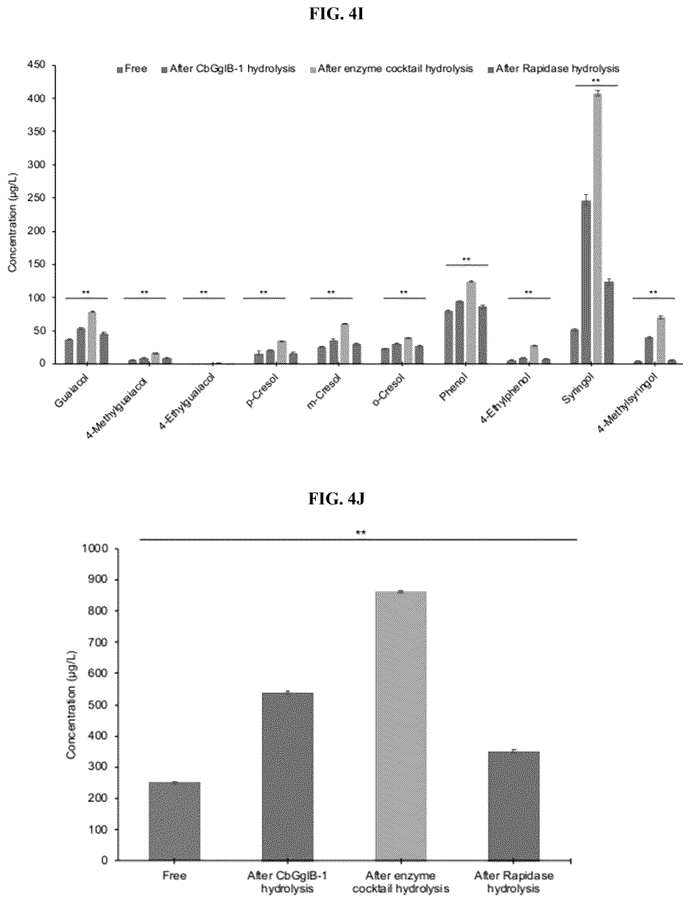

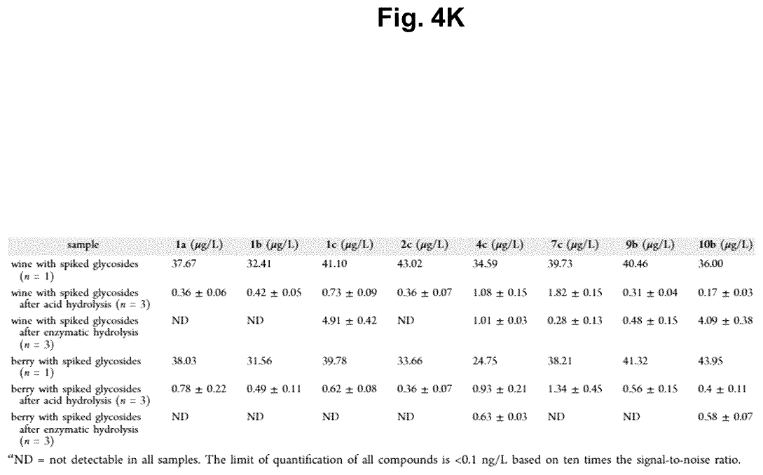

Smoke-associated volatiles levels have been identified, for example, after treatment with the enzymes described herein, for example, as described below. See, e.g., FIG. 4E.

### EXAMPLES

### Example 1

### Identification of Active Glycosidases on Guaiacol Glycosides Through Genome Mining

To identify enzymes with the ability to cleave glycosidic bonds in bound volatile phenols (VPs), the sequence space of the glycosidase 1 (GHI) enzyme family was explored through genome mining in a gene sequence database, UniProt and NCBI GenBank.

The approach involved collecting and characterizing an assortment of representatives from the gene sequence database that would capture a considerable amount of sequence diversity within the targeted enzyme family. GHIs catalyze the hydrolysis of B1-4 bonds and the GHI enzyme family is widely distributed in archaea, eubacteria, and eukaryotes. The GHI family was chosen as the primary target because GHIs have diverse substrate specificities on both conjugated sugars and aglycons. Recently, a comprehensive examination of the functional variety within this group of enzymes further validates GHI substrate promiscuity and its suitability for industrial purposes.

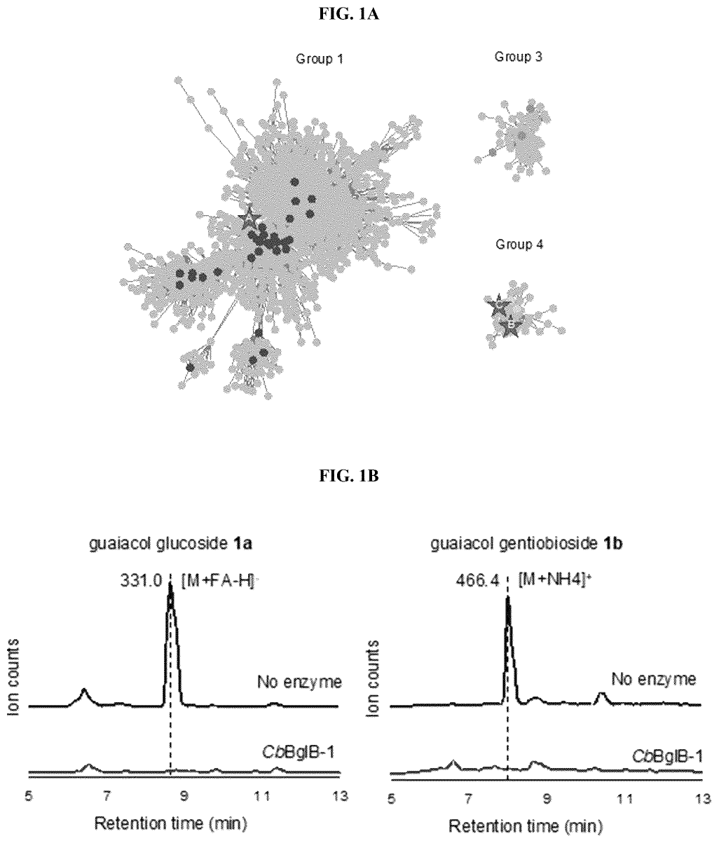

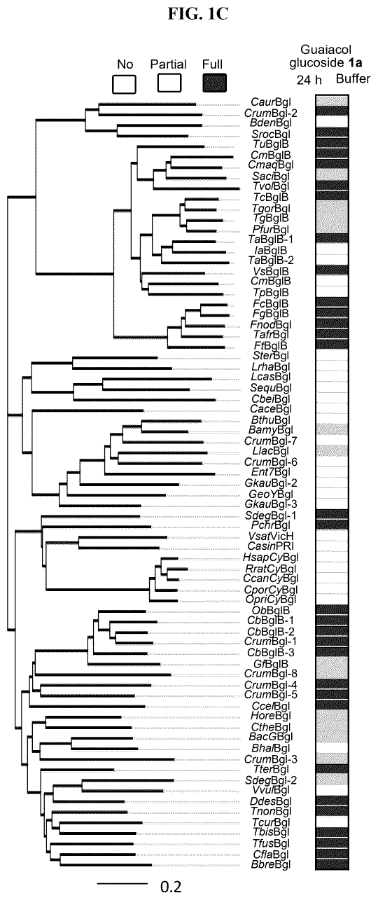

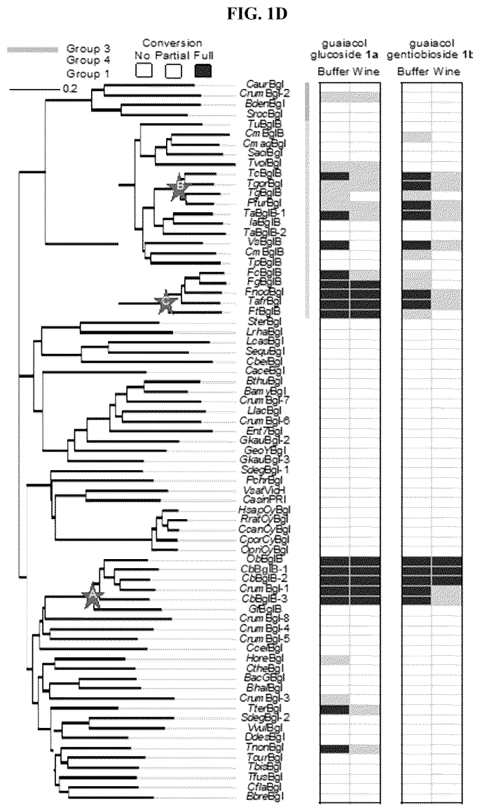

A total of approximately 80,000 genes presumably annotated as the GHI family were visualized via sequence similarity network (SSN) based on their phylogenetic relationships, in which all sequences sharing 75% or more identity were grouped into a single meta node (Rep node). A set of 73 synthetic genes encoding naturally occurring proteins were procured (FIG. 1A). Only the groups containing the tested sequences are depicted in FIG. 1A. The 73 genes were distributed within the clusters of group 1 (49/73), group 3 (4/73) and group 4 (20/73), ranked by the total number of genes represented, and the three groups accounted for more than 70% of sequences in GHI family. The most active GHs located in representative nodes A in group 1 and B, C in group 4. The collection of genes represents a considerable diversity in sequence space with an average identity of 30% to each other.

Synthetic genes encoding the 73 proteins were purchased, cloned into a pET29b+vector with a C-terminal 6× histidine tag (SEQ ID NO: 79), and overexpressed in E. coli. The corresponding proteins were purified by IMAC and analyzed by SDS-PAGE. The obtained enzymes underwent stepwise testing to evaluate the ability to release VPs and the activity was semi-quantitatively assessed based on the degree of substrate disappearance post-reaction by LC-MS (FIG. 1B). This figure demonstrates the application of this method using ChBg1B-1 as a. representative example. The semi-quantitative activity was evaluated by comparing ion counts in MS between samples with the added enzyme and those without it.

Initial proof of concept studies were acetic acid buffer conditions at pH 3.5 with 4.5 mg/L guaiacol glucoside (compound 1a) as the substrate at 37° C. over a 24-hour period. 45/73 enzymes were found to be active towards compound 1a while the other 28 enzymes were either inactive or not expressed in a soluble form (FIG. 1C).

The enzymes were then tested under acetic acid buffer conditions at pH 3.5 and baseline Cabernet Sauvignon (no pH adjustment) and a 4-hour incubation time. The enzyme activity in both Systems were compared because it is well known that the chemical compounds in wines, especially in red wines, such as ethanol, glucose, tannins, and metals can inhibit GHs, and the side-by-side comparison can provide the necessary information to determine whether the lack of activity in wine was due to inhibition. For guaiacol glucoside (compound 1a), 22 enzymes exhibited glycosidase activity out of which 15 were capable of completely catalyzing the release of guaiacol in an acetic acid buffer (FIG. 1D). Candidates such as CbBg1B-1 were mixed with baseline wine which had been spiked with 4.5 mg/L each of compounds 1a and 1b. The reaction was at 37° C. for 4 hours' duration. As for guaiacol gentiobioside (compound 1b), 18 enzymes were active, with 12 of them able to fully catalyze the liberation of guaiacol in an acetic acid buffer. It was noted that the activity is focused on the enzymes in Ref50 clusters (highlighted as stars) of AOA4P2Q3W9 in group 1, P22498 and A0A1E3G457 in group 4.

Inhibition in Cabernet Sauvignon was clearly observed for both substrates. Among the 12 enzymes that can fully utilize compound 1a in acetic acid buffer, 9 enzymes maintained complete functionality. However, in the case of compound 1b, only 3 enzymes completely catalyzed the release of guaiacol in Cabernet Sauvignon, namely Bg1b from Oscillospiraceae bacterium (ObBg1B), Bg1B-1 (CbBg1B-1) and Bg1B-2 (CbBg1B-1) from Clostridia bacterium. These three enzymes also demonstrated shared activity towards compound 1a, indicating a potential functional overlap in their ability to catalyze the release of volatile phenols. All three enzymes are from Clostridía bacteria class in ruminant gastrointestinal microbiome and share about 70% sequence identity to each other. This represents the first instance where these three enzymes have been characterized against smoke associated phenolic glycosides.

### Example 2

### Characterization of CbBg1B-1

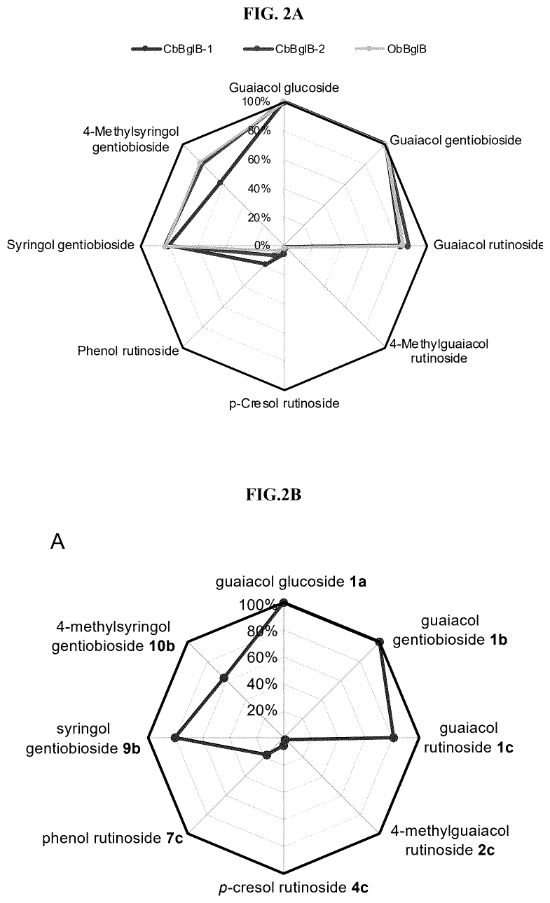

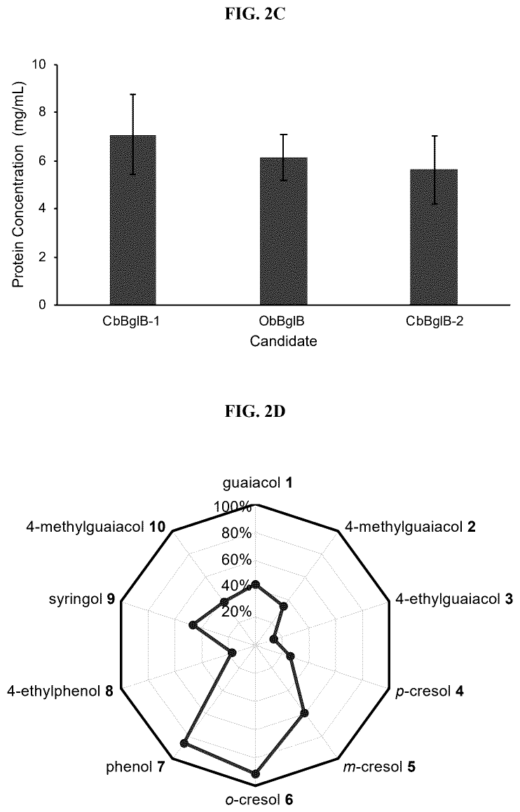

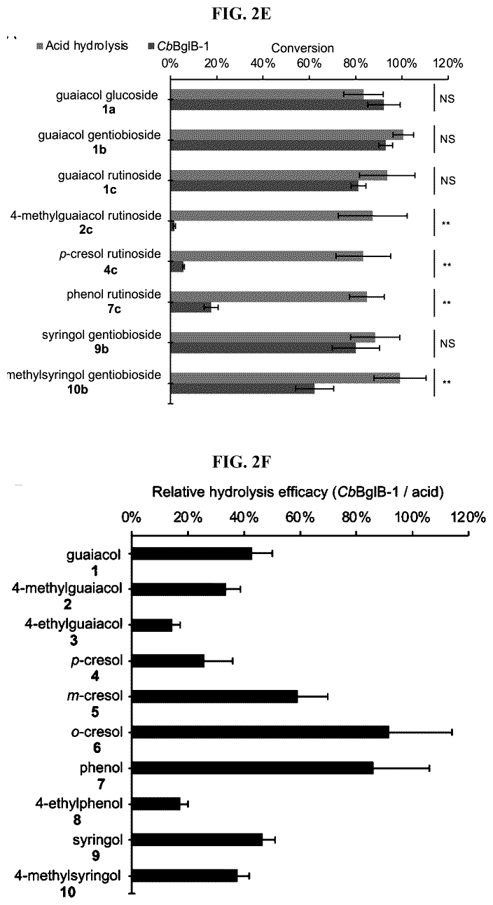

To select the best candidate among the three outstanding enzymes in the initial screening, the actives and substrate scopes of the enzymes were compared with fortification experiments. 8 commercially available P-D-glycosides namely guaiacol glucoside (compound 1a), guaiacol gentiobioside (compound 1b), guaiacol rutinoside (compound 1c), 4-methylguaiacol rutinoside (compound 2c), p-cresol rutinoside (compound 4c), phenol rutinoside (compound 7c), syringol gentiobioside (compound 9b), 4-methylsyringol gentiobioside (compound 10b) with diverse VP aglycons and sugar moieties were spiked in baseline Cabernet Sauvignon with a more realistic concentration of 40 μg/L at 37° C. for 4 hours. The conversion value is calculated by subtracting the final concentration of each VP in baseline wine from those after enzymatic hydrolysis, then dividing by the theoretical mass of each VP. The conversion rate is determined based on the concentration of VPs recovered through enzymatic hydrolysis, as quantified by GC-MS. Similar substrate scope and activity profiles were observed for ObBg1B and CbBg1B-2. All three enzymes can utilize more than 80% of guaiacol glycosides namely compound 1a, compound 1b and compound 1e as expected and about 80% of compound 9b (FIG. 2A). All three enzymes displayed a strong preference on gentiobioside b. Whereas ObBg1B and CbBg1B-2 resulted in higher compound 10b conversion, CbBg1B-1 could utilize compound 7c exclusively (FIG. 2B and FIG. 2E). The result showed that CbBg1B-1 displayed minor activities towards VPs rutinosides. All proteins were expressed in E. coli in 500 mL Terrific Broth culture, purified through cobalt IMAC and quantified through A280. CbBg1B-1 shows markedly higher expression level than ObBg1B and CbBg1B-2, which is potentially beneficial for industrial applications (FIG. 2C). Therefore CbBg1B-1 was selected for as the protein of interest for subsequent testing and optimization.

To evaluate performance of CbBg1B-1 in a previously validated sample of smoke-tainted wine, a direct comparison was performed between acid hydrolysis and CbBg1B-1 mediated enzyme hydrolysis from phenolic glycosides in a smoke-tainted Cabernet Sauvignon. Using the levels of phenolic glycosides generated by acid hydrolysis as a benchmark, we can calculate the ratio of each glycoside converted by enzymatic hydrolysis relative to acid hydrolysis. The ratio for each VP was calculated by dividing the total VP release measured after enzymatic hydrolysis by that of acid hydrolysis. A value greater than 100% would imply that enzymatic hydrolysis is more accurate of total VP in the matrix than acid hydrolysis, while a value less than 100% would suggest the opposite. Triplicate data were collected, and averages reported, all standard deviations were <10%. Enzymatic hydrolysis achieved less than 90% conversion for the majority of the measured VPs compared to acid hydrolysis, with the majority of VPs between 20% to 50% of the conversion yields observed in acid hydrolysis (FIG. 2D and FIG. 2F). CbBg1B-1's activity levels were also found to be sensitive to the type of aglycon present. This was illustrated by the enzyme's high activity on compound 1c, contrasted with its significantly lower activity on compounds 2c, 4c, and 7, despite the tested compounds (1c, 2c, 4c, and 7c) sharing the same rutinoside motif. Comparing this data to the high-yield observed in simulated smoke-taint data indicated that while CbBg1B-1 is efficacious at releasing glucosides and gentiobiosides, it has a low efficacy in releasing rutinosides. Therefore, additional genome mining efforts would be required to find a synergistic enzyme capable of releasing rutinoside-bound VPs. The direct comparison between acid hydrolysis and CbBg1B-1 showed that although similar efficacy on guaiacol glycosides 1a, 1b and 1c was observed in fortified samples (FIG. 2E), a lower efficacy of CbBg1B-1 compared to acid hydrolysis was noted in real-world samples (FIG. 2F). This may be attributed to the presence of other guaiacol glycosides as well as potential substrate and product inhibition.

### Example 3

### Identification of Active Rutinosidases on Phenolic Rutinosides Through Genome Mining

The 6-O-a-L-rhamnopyranosyl-b-D-glucosidases (rutinosidases; EC 3.2.1.168) belong to the GH 5 subfamily 23 and specifically act on the flavonoid diglucoside, including compounds like quercetin 3-O-rutinoside, hesperetin 7-O-rutinoside, Kaempferol-3-O-rutinoside, and naringenin 7-O-neohesperidoside. Notable rutinosidases have been reported from several species, including Acremonium sp. DSM 24697, Actinoplanes missouriensis, Aspergillus niger K2, and Aspergillus oryzae Rfl340. Advancements have been made recently in understanding the properties of these enzymes and the crystal structures of rutinosidase from Aspergillus niger K2 (AniRut), and rutinosidase from Aspergillus oryzae RIB40 (AoryRut) were deciphered to shed light on the substrate specificity. Remarkably, AoryRut is capable of accommodating various flavonoids including both 7-O-linked and 3-O-linked flavonoids, possibly contributed by the flexible loop located at the substrate entrance. While there's considerable interest in its application within the food industry, the exploration of the enzymes' substrate scope beyond flavonoid glycosides remains limited. Genome mining was performed in non-exhaustive manner with a particular emphasis on identifying rutinosidase activity against 4-methylguaiacol rutinoside compound 2c among the collection of selected proteins.

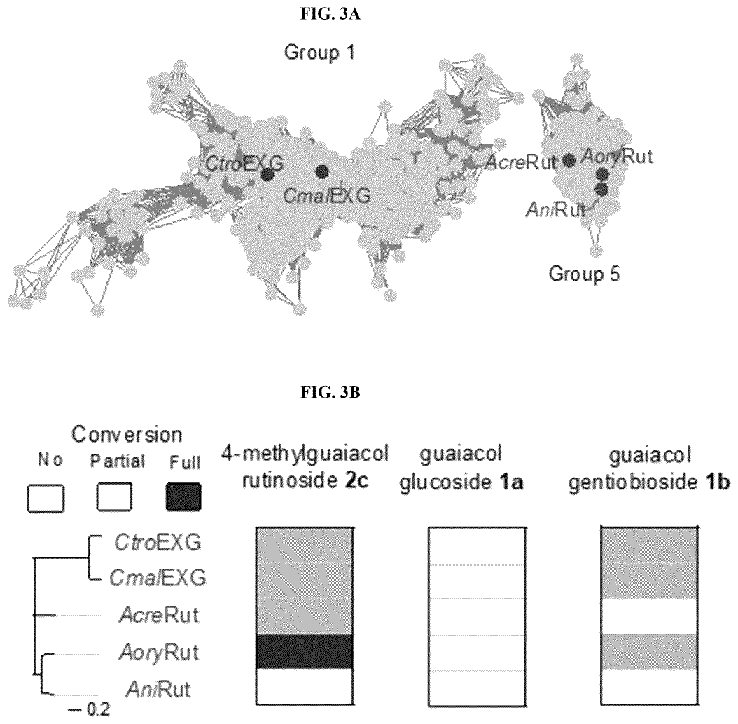

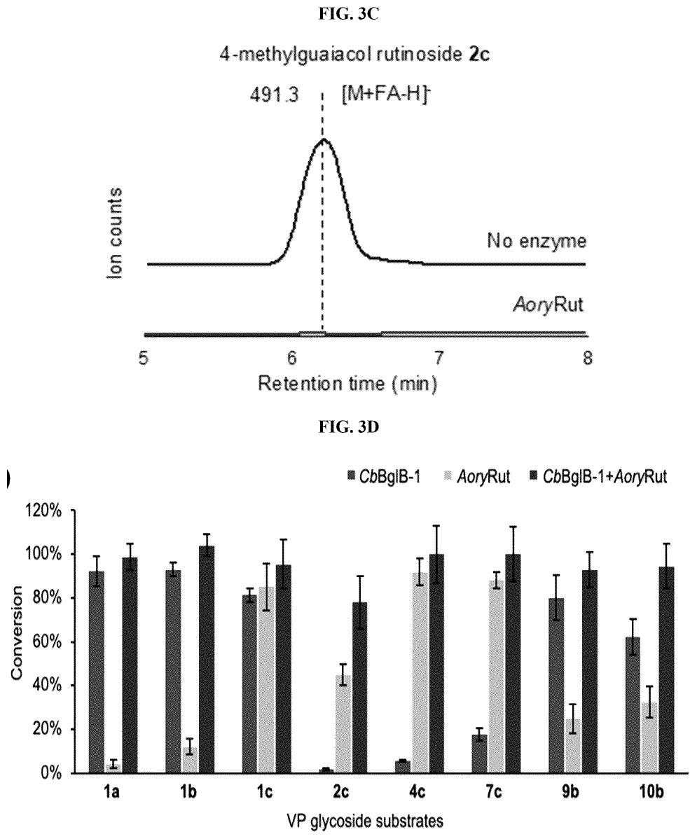

GH5 SSN composed of about 67,000 genes was built and previously identified rutinosidases such as AoryRut and AniRut centered on group 5. A higher preference was assigned to enzymes situated in group 1 and group 5 to ensure that the chosen representatives spanned across a wide sequence space, while also leveraging the accessible knowledge base (FIG. 3A). The genes encoding CtroEXG, CmaJEXG, AcreRut, AoryRut and AniRut with average sequence identity around 50% were selected, and their corresponding proteins expressed in E. coli were purified. Candidates were mixed with baseline wine which had been spiked with 4.5 mg/mL of compound 2c. The reaction is at 37° C. for 4 hours' duration and their semi-quantitative performance on compound 2c were evaluated by LC-MS. While 4 out of 5 showed activity, AoryRut was the sole enzyme that could fully use compound 20 (FIG. 3B, FIG. 3C). Their ability to utilize compound 1a and compound 1b was also examined, and the result showed that 3 out of 5 were active towards compound 1b but none of them were active on compound 1a (FIG. 3C). The result was consistent with previous report that AoryRut demonstrated different substrate promiscuity to AniRut and the specificity is determined by both glycome types in flavonoid glycosides and the aglycone moiety, and generally prefers disaccharide glycosides to monosaccharide glycosides. AoryRut could completely degrade compound 2c indicated by the disappearance of the corresponding peak in MS traces.

CbBg1B-1 is annotated as a GHI enzyme family in which the enzymes typically exhibit exacting activity with the progressive release of monosaccharides from these linkages. AoryRut has been classified as a GH5 diglycosidase and can cleave the entire disaccharide moiety from the aglycone. The obtained activity profile of AoryRut underscores that AoryRut can serve as an effective complement to CbBg1B-1 for the purpose of maximizing the release of phenolic glycosides. When the enzyme cocktail of CbBg1B-1 and AoryRut was employed, the synergetic effects led to the additive enhancement on harnessing the full spectrum of glycosides (FIG. 3C). Remarkably, the combination achieved more than 90% conversion on nearly all tested glycosides, except for compound 2c, which is around 80% conversion. By strategically combining enzymes of CbBg1B-1 and AoryRut with verified modes of action, it became possible to target a broader range of glycosidic bonds and is likely to yield diversified glycosidic bond cleavage in smoke-derived VP glycosides (FIG. 3D; in FIG. 3D, first bar for each glycoside is the sample treated with CbBg1B-1, the second bar for each glycoside is sample treated with AoryRut and the third bar is the combination of enzymes), Thus, the enzyme cocktail is a promising candidate for comparison against the conventional acid hydrolysis approach.

### Example 4

### Hydrolysis Efficacy Comparison Between Enzymatic Hydrolysis and Acid Hydrolysis

To establish the optimal parameters for enzymatic hydrolysis, that directly affect the process of enzymatic hydrolysis two notable parameters were examined, namely, incubation time and enzyme loading. To fine-tune the incubation time, various reaction durations including 0.25 hours, 1 hour, 4 hours and 24 hours were tested. Time-course experiment indicated that the reaction achieved equilibrium in 4 hours and the extension of reaction time would not necessarily yield more VPs (FIG. 4A). To determine the best enzyme loading value, the high smoke-impacted Cabernet Sauvignon was mixed with varying ratios and concentrations of constituent enzymes in the cocktail. CbBgIB was first assessed with five different loading amounts, resulting in five varying final enzyme concentrations of CbBg1B-1 (0.4 mg/mL, 0.8 mg/mL, 2 mg/mL, 4 mg/mL., 5 mg/mL) and compared the outcomes of total VPs. While the higher concentration of CbBg1B-1 up to 4 mg/mL resulted in increasing summed amount of VPs, there was no significant difference when comparing the results using 4 mg/mL and 5 mg/mL enzyme (FIG. 4B). 4 mg/mL of CbBg1B-1 was thus applied to the following experiments with the assumption that loading more than 4 mg/mL of ChBg1 B-1 would not generate more VPs in the matrix of present smoke-tainted wine. Various concentrations of AoryRut: 0.2 mg/mL, 0.5 mg/mL, 0.8 mg/mL, 1.0 mg/mL and 1.2 mg/mL were tested in combination with 4 mg/ml of CbBg1B-1. The quantity of total VPs increased along with the concentration of AoryRut, up to maximum of 1.0 mg/mL. Higher concentration of AoryRut than 1.0 mg/mL did not make a significant difference in total VP levels (FIG. 4C). Overall, the enzyme cocktail operated when the incubation time was at least 4 hours and the concentrations of CbBg1B-1 and AoryRut were 4 mg/mL and 1 mg/mL, respectively.

A comparative study of hydrolysis using glycosidase 2 (Rapidase Revelation Aroma), CbBg1B-1, and AoryRut was done (FIG. 4I and FIG. 4J). FIG. 4I denotes individual VP concentration before (Free) and after enzymatic hydrolysis of high smoke-impacted wine. FIG. 4J depicts the sum of VPs concentration before (Free) and after enzymatic hydrolysis of high smoke-impacted wine. Biological triplicates were performed. In FIG. 47, rapidest indicates DSM Rapidase Revelation Aroma with final concentration of 0.03 g/L in samples. In FIG. 4I and FIG. 4J** denotes statistically significant with p-value <0,05. While glycosidase 2 increased the concentration of all free VPs, its activity was significantly lower than that of CbBg1B-1, with the total VP concentration reaching only about 65% of that produced by ChBg1B-1-catalyzed reactions. The final accumulated concentration of VPs catalyzed by glycosidase 2 was approximately 40% of that achieved by a cocktail of CbBg1B-1 and AoryRut. Thus, glycosidase 2 exhibited suboptimal activity for VP glycoside quantification and might not be directly used for this purpose without additional optimization.

To further corroborate the efficacy of the enzyme cocktail, a direct quantification strategy for VP glycosides in wine and berries was implemented, Nonsmoke-affected samples were mixed with known VP glycoside substrates and then conducted LC-MS/MS analysis both before and after subjecting them to enzymatic and acid hydrolysis. This method allowed the measurement of the conversion of VP glycosides accurately. The results confirmed that both acidic and enzymatic hydrolysis successfully converted all VP glycosides (FIG. 4K). In wine, enzymatic hydrolysis showed slightly enhanced effectiveness over acid hydrolysis for substrates 1a, 1b, 2c, 4c, and 7c, though it was less efficient for 1c, 8b, and 10b. Enzymatic hydrolysis achieved a minimum conversion rate of 88% in wine for all VP glycosides. For grape samples, enzymatic hydrolysis generally yielded higher conversion rates for almost all VP glycosides with 10b being the sole exception, The direct measurement of the depletion of VP glycosides was consistent with the formation of free VPs, thus reinforcing the validity of the approach.

Enzymatic hydrolysis catalyzed by the enzyme cocktail after formulation optimization was then carried out in Cabernet Sauvignon wines and grape berries that were divided into two categories: smoke-impacted and non-smoke-impacted. Both acid hydrolysis and enzymatic hydrolysis demonstrated significantly higher total VPs concentrations in smoke-impacted wine and grape than those in non-smoke-impacted samples. Reflected by the total concentration of VPs, both wine and grape samples impacted by smoke contained significantly elevated concentrations of phenolic glycosides compared to those samples unaffected by smoke, and the results validated the potential of hydrolysis method for binary and qualitative assessments of smoke impact (FIG. 4D, FIG. 4E). Among the phenolic glycosides, glycosides of syringol (compound 9a, compound 9b, compound 9c) calculated from the subtraction of Free 51.17 μg/L from Total (after hydrolysis) 407,7 ρg/L were the most abundant in smoked-impacted Cabernet Sauvignon with the concentration of 356.5 μg/L (FIG. 4E). Compound 9b was one of the predominant glycosides in high smoke-tainted Cabernet Sauvignon and our result is in accordance with prior studies. The concentrations of compound 3a, b, c and compound 8a, b and c in smoke-impacted wine after enzymatic hydrolysis were approximately 10-fold higher than those in the baseline, which showed that compound 3a, b, c and compound 5a, b, and c which are normally associated with Brettanomyces yeast growth, can also be present as a consequence of smoke exposure. Compound 1 a, b, and c and compound 2a, b, c which are typically regarded as markers of smoke taint exhibited a significant increase following enzymatic hydrolysis and their concentrations were clearly distinguishable between smoke-impacted samples and non-smoke-impacted samples.

A detailed analysis was conducted to compare the differences between enzymatic hydrolysis and acid hydrolysis in wine samples. The enzymatic hydrolysis led to a higher conversion of half of the bound VPs in both smoke-impacted and non-smoke-impacted wines, albeit for different VPs (FIG. 4F), Enzymatic hydrolysis significantly outperformed acid hydrolysis for compound 5, 6 and 7 (a, b, and c) with the range of 150%-300% higher conversion. The enzymatic hydrolysis displayed a comparable effectiveness for compound 1, 2, 4, 8, 9 and 10 (a, b, c) albeit varying ratios seen in the smoked and unsmoked wines. It's worth mentioning that aligned with the established literature, it was found that syringol compound 9 (a, b, and c) and 4-methylsyringol compound 10 (a, b, and c) were effectively released by both acid hydrolysis and enzymatic hydrolysis.

To alleviate the economic consequences of producing smoke-affected wines, it is useful to determine the quantities of both free and bound VPs in grapes prior to fermentation. As part of this initiative, enzymatic hydrolysis of smoke-impacted Cabernet Sauvignon grapes and control grapes was studied. This allowed us to assess the method's compatibility with grapes, which are more challenging to accurately determine VPs under acid hydrolysis conditions. Following a similar trend as observed in smoke-impacted wine, total VPs in post-hydrolysis of smoke-impacted grape berries were considerably higher than control grape, and compound 9 persisted as the most abundant VP after hydrolysis in smoke-impacted grape berries (FIG. 4E), Fermentation by yeast and the aging process can hydrolyze the bound VPs while the lack of glycosidase activity in grapes may slower the transfer of bound VPs from grapes into wine, indicating that smoke-exposed grape samples should theoretically contain a greater proportion of bound VPs and result in a higher ratio of bound to free VPs in grapes compared to wine. The findings herein supported this theory, as a notable increase in the ratio of bound to free VPs in smoke-impacted grapes than wines was observed.

Consistent with the performance in wine samples, enzymatic hydrolysis showed 150%-300% increase of conversion than acid hydrolysis for bound forms of compound 5, 6 and 7 (a, b, c) (FIG. 4G). Interestingly, enzymatic hydrolysis substantially excelled for the glycosides of compound 8 (a, b, c) in both types of grape samples, whereas its performance was only marginally superior in smoke-impacted wine samples. The conversion rate for all other VPs between enzymatic hydrolysis and acid hydrolysis were nearly identical despite minor increase of enzymatic hydrolysis for bound compounds 3 and 4 (a, b, c). It was noted that the ratios of enzymatic hydrolysis to acid hydrolysis for all phenolic glycosides exhibited less variation in smoke-impacted and non-smoke-impacted grapes than in wine samples, illustrating the operational stability in grapes. Finally, relative hydrolysis efficiencies of enzymatic to acid for individual bound VPs were mapped into box and whisker plots to summarize the value distribution across different sample types (FIG. 4H). The median and mean values of the relative efficacy for VP glycosides in both wine and grape samples are >1.0. The relative hydrolysis efficacy in both wine and grape samples are not statistically different, indicating the enzymatic hydrolysis method has consistently higher hydrolysis efficacy than acid hydrolysis regardless of the sample types. In FIG. 4H, NS denotes not significant (the two-tailed P value >0.5). Experiments were conducted in triplicate. The enzymatic hydrolysis method consistently demonstrated more effectiveness compared to acid hydrolysis across all tested bound VPs in both wine and berry samples with the approximate median of 1.2 and mean of 1.35. Moreover, the enzymatic hydrolysis method demonstrated near-identical performance regardless of the degree of smoke impact, showcasing the robustness and consistency of the enzymatic hydrolysis approach.

Utilizing enzymatic hydrolysis has the potential to bring several notable advantages. First, enzymatic hydrolysis surpasses acid hydrolysis in efficacy. Second, acid hydrolysis is well known to be sensitive to conditions and handling, making it difficult to standardize across laboratories. Conversely, enzymatic hydrolysis operates under milder conditions and avoids the use of harsh chemicals. This provides a safer work environment, a useful consideration in laboratory settings. Third, the reduced sample preparation such as pH titration, makes enzymatic hydrolysis an efficient choice for high-throughput. This high-throughput capability is particularly beneficial for grape growers and wine makers, allowing for prompt decision-making, especially during fire seasons. Fourth, the method is cost-effective and eliminates the need for high cost and low throughput LC-MS/MS based analytics.

### Example 5

### Materials and Methods:

Bacterial strains, plasmids, and chemical reagents

The bacterial strain used for cloning was Escherichia coli DH5a; the pET29 (+b) plasmids containing the protein encoding genes were expressed in E. coli BLR (DE3). All genes were purchased as synthetic genes optimized for E. coli codon usage with infusion of 6-histidine at the C-terminus. The sequences of genes encoding all glycosidases in the present work are listed in Table 2 and Table 3.

Grape and wine samples. The grapes used for this study were sourced from Vitis vinifera L. cv. Cabernet Sauvignon from California with a significant smoke impact in 2020. And the high-smoke-impacted Cabernet Sauvignon were obtained from simulated smoke exposed vinifera L. cv. Cabernet Sauvignon.

SSN and Sequence analysis

SSN was built by EFI-EST web-tool and visualized in Cytoscape. The Interpro IPR001360 collection of GHI enzyme sequences combined with JGI IMG Integrated Microbial Genomes & Microbiomes database annotated GHI enzymes were used as the input for EFI-EST analysis of GHI while Interpro IPR001547 annotated as rutinosidase were used as the input for GH5. For both of SSN, only Ref50 clusters were used. Sequence identity threshold of 45 was used as parameter for filtering the sequences into clusters in SSN and representative node networks with 70% identity were displayed.

### Protein Expression and Purification

E. coli was first grown overnight as the starter culture at 37° C. in Terrific Broth medium (1% tryptone, 0.5% yeast extract, 0.5% NaCl) supplemented with Kanamycin (50 μg/mL final concentration) and MgSO₄ (1 mM final concentration). The culture for protein expression was diluted by −50-fold to 500 mL from the starter culture. The cultures were then grown until OD600 to −0.6 at 37° C., and IPTG was supplemented to final concentration of 0.5 mM for induction at 16° C. for 24 h. At the end of induction, cells were centrifuged (4,700×g., 4° C., 10 min), supernatant was removed, cells were resuspended in 40 mL lysis buffer (50 mM HEPES, pH 7.0, 300 mM NaCl, 10% glycerol, 1 mM MgSO₄, 15 mM imidazole), and sonicated for 2 min at 4° C. Lysed cells were centrifuged at 4,700×g at 4° C. for 30 min to remove cell debris. Supernatant was loaded on a gravity flow column with 1 mL of cobalt slurry, which was pre-balanced with 30 mL of wash buffer (50 mM. HEPES, pH 7,0, 300 mM NaCl, 10% glycerol, 1 mM MgSO₄, 15 mM imidazole). The cobalt resin was then washed three times with 10 mL wash buffer, proteins were eluted with 0.6 mL of elution buffer (50 mM HEPES, pH 7.0, 300 mM NaCl, 10% glycerol, 1 mM MgSO₄, 1 mM TCEP, 200 mM imidazole). Protein samples were immediately buffer exchanged with spin concentrators into storage buffer (50 mM HEPES, pH 7.0, 300 mM NaCl, 10% glycerol, 1 mM MgSO₄) and stored at 4° C. until activity characterization. Protein concentrations were determined using a spectrophotometer by measuring absorbance at 280 nm using their calculated extinction coefficients. The protein samples were further analyzed by 12% SDS-PAGE gel

### Initial Activity Screening by Liquid Chromatography Mass Spectrometry (LC-MS)

Purified enzymes were added into both buffer and baseline wine samples with substrates 1a, 1b and 2c spiked in. The reaction mixture was kept at 37° C. for 24 hours or 4 hours. After cooling down on ice, the reactions were quenched by adding to 50% volume of acetonitrile then centrifuged. The supernatant was subjected to activity assay.

Reverse-phase high-performance liquid chromatography and mass spectrometry (LC-MS) for analysis were carried., The gas temperature was 350° C., drying flow was 13.0 L/min, and capillary voltage was 4300 V. Each sample was analyzed in triplicate. The mobile phase consisted of the following gradient: 70% H₂O with 0.1% formic acid as mobile phase A and 30% ACN with 0.1% formic acid as mobile phase B for 5 mins; 10% mobile phase A and 90% mobile phase B from 8 to 19 min; mobile phase A was decreased to 70% with 30% mobile phase B until 25 min. The HPLC flow rate was 0.5 mL/min and the injection volume was 3 μL. The parameter of the mass spectrum was adjusted accordingly for different glycosides as shown in FIGS. 2 and 4.

### Acid Hydrolysis and Enzymatic Hydrolysis

Sample prep for grape berries: Samples were removed from the freezer, then 65 g of berries were separated from cluster rachi, taking care to prevent berry cap and other non-berry debris from introduction into the sample container. Samples were thawed for 15-20 minutes at room temperature, 15 mL water was added to the sample, homogenized with a high-speed commercial blender for 1 min, paused for 1 min and then homogenized for a further 30 s.

Enzymatic hydrolysis: 4 g of the homogenized berry sample or 4 mL of wine were transferred into 20 mL GC vials purchased from Agilent. 16 μL of ethanolic d3-guaiacol (5 mg/L) internal standard was added to samples (final concentration of 20 μg/kg in berry homogenate or 20 g/L in wine), Glycosidase enzymes were then added to the samples. For enzymatic hydrolysis of real-world samples, the final concentrations of 4 mg/mL and 1 mg/mL of CbGg1B-1 and AoryRut were added, respectively. The reactions were conducted at 37° C. for 4 hours.

Acid hydrolysis: Samples were aliquoted into 20 mL glass tubes in 10 mL and the pH was adjusted to 1.0 with 4M HCl then spiked with 40 μL of ethanolic d3-guaiacol (5 mg/L) internal standard.

Samples were then transferred from the glass tubes to 17 mL Teflon tubes equipped with tightly fitted caps. Samples were incubated at 100° C. for 1 hour, then cooled over ice for 10 min before aliquoting 4 mL wine or 4 g grape homogenate into GC vials.

Quantitative HS-SPME GC-MS analysis.

HS-SPME: Smart SPME arrow 1.1 mm DVB/CarbonWR/PDMS (Agilent 5610-5861) was used by PAL3 robotic autosampler for sample injections. The SPME headspace settings: predesorption time: 4 min and temperature: 250° C. Sample incubation time: 4 min. Sample vial penetration depth: 35 mm. Inlet penetration depth: 40 mm. Inlet penetration speed: 100 mm/s.

Sample vial penetration Speed: 35 mm/s. Sample extraction time: 9 min and extraction temperature: 60° C. Heatex stirrer speed: 1,000 rpm and temperature: 40° C. Sample desorption time: 3 min.

GC-MS:. All samples in 20 mL GC-MS headspace vials ready to assay were added with 40% w/v NaCl. The GC-MS injection mode was splitless at 250° C. GC has a constant flow of 1.2 mL/min helium gas. The oven program was 120° C. (hold 1 min); 9° C./min to 250° C. (hold 0 min); 250° C./min to 280° C. (hold 0 min). The guard chip temperature was 200° C., bus temperature 280° C. and MSD transfer line 280° C.

### Statistical Analysis

All experiments were independently carried out in triplicate. The differences between samples were evaluated by student's t-test. The P values <0.05 indicates statistically significant difference.

### Example 6

### Removal of Volatile Phenols

Following the enzymatic hydrolysis reactions described in Examples 1-4, volatile phenols are removed from fruit products or fermented fruit products such as wine using methods known in the art. Volatile phenols can be removed by available techniques, such as using (i) activated carbon by filtration or reverse osmosis, (if) using yeast lees or cells walls, (iii) using enzymes, (iv) using cellulose, (v) using cyclodextrins polymers, and/or (vi) using molecularly imprinted polymers.

### Example 7

### Rutinosidase Enzyme Engineering for Increased Expression and Stability

The computational enzyme design software Rosetta suite, which includes algorithms for computational modeling and analysis of protein structures was applied. Residues distal to the active site (>8 Å) were targeted for mutations to avoid potential activity disruption due to engineering. Each position was designed by Rosetta using a position-specific substitution matrix (PSSM) constructed from sequence alignment of the entire rutinosidase enzyme family. Only mutations with a favorable PSSM score (=>0) were chosen as targets. The selected mutations were then subjected to in silico mutation and further evaluated using Rosetta score terms. The top 50 designs with the lowest total scores were selected as potential candidates for further evaluation. The structures of these 50 designs were built using Rosetta and visualized in PyMOL software. Evaluation involved chemical intuition to remove obviously unreasonable designs, focusing on those that presumptively increase protein packing (e.g., small residue to large residue, non-polar residues to polar residues to introduce new hydrogen bonds). Ultimately, 22 designs (MC4-MC25) were constructed and screened. Beneficial mutations for protein expression were then combined to obtain MC52-MC60 for further screening.

To identify AoryRut (SEQ ID NO: 73) was mutated and the resulting mutants were screened to identify mutations that increase expression and enzyme stability while maintaining enzymatic activity. Table 4 shows the mutants and combination of mutants selected for screening. The AoryRut mutants were introduced into Escherichia coli (E. coli) and expression of the enzymes was measured. Table 4 shows the expression level of the AoryRut mutants. AoryRut mutants MC8 (T141V), MC14 (S184F), MC15 (M190D), MC21 (Q307N), MC55 (T14IV, T214A, Q307N). MC56 (T141V, M190I, Q307N), MC58 (M190I, T214A) showed expression greater than AoryRut. Among the different mutants screened, AoryRut mutant MC56 having mutations at positions T141V, M190I and Q307N showed highest expression in E. coli.

TABLE 4
AoryRut mutant expression

| | | Expression Level |
|---|---|---|
| | | (mg/mL per 500 mL |
| Enzyme Name | Mutation | culture) |
| AoryRut | N/A | 0.24 |
| MC4 | Q38D + F39W + G41N | N/A |
| MC5 | G87N | 0.2 |
| MC6 | T94N | 0.11 |
| MC7 | T141I | 0.21 |
| MC8 | T141V | 0.44 |
| MC9 | T145V | 0.16 |
| MC10 | Y156F | 0.21 |
| MC11 | V168M | 0.25 |
| MC12 | S181Y | 0.14 |
| MC13 | Q183W | 0.69 |
| MC14 | S184F | 0.38 |
| MC15 | M190I | 0.69 |
| MC16 | T214A | 0.16 |
| MC17 | N270R | 0.22 |
| MC18 | L276K | 0.17 |
| MC19 | R279H | 0.44 |
| MC20 | T297V | 0.13 |
| MC21 | Q307N | 0.44 |
| MC22 | M324W | 0.17 |
| MC23 | M324W, S328T | 0.11 |
| MC24 | S328T | 0.15 |
| MC25 | A342F | 0.21 |
| MC52 | T141V, M190I | 0.31 |
| MC53 | T141V, T214A | 0.2 |
| MC54 | T141V, Q307N | 0.3 |
| MC55 | T141V, T214A, Q307N | 0.28 |
| MC56 (SEQ ID | T141V, M190I, Q307N | 1.12 |
| NO: 78) | | |
| MC57 | T144V, M190I, T214A, | 0.11 |
| | Q307N | |
| MC58 | M190I, T214A | 0.34 |
| MC59 | M190I, Q307N | 0.1 |
| MC60 | M190I, T214A, Q307N | 0.26 |

The stability of AoryRut mutant MC56 (SEQ ID NO: 78) and having mutations at positions T141V, M190I and Q307N relative to SEQ ID NO: 73 was analyzed. The results are shown in Table 5. The stability analysis showed that MC56 has greater stability than wild type AoryRut of SEQ ID NO: 73.

TABLE 5
AoryRut mutant stability

| Expression Level | |
|---|---|
| (Per 500 mL culture) | Melting Temperature (° C.) |
| MC56 (SEQ ID NO: 78) | 55.6 |
| H1 (SEQ ID NO: 73) | 54.5 |

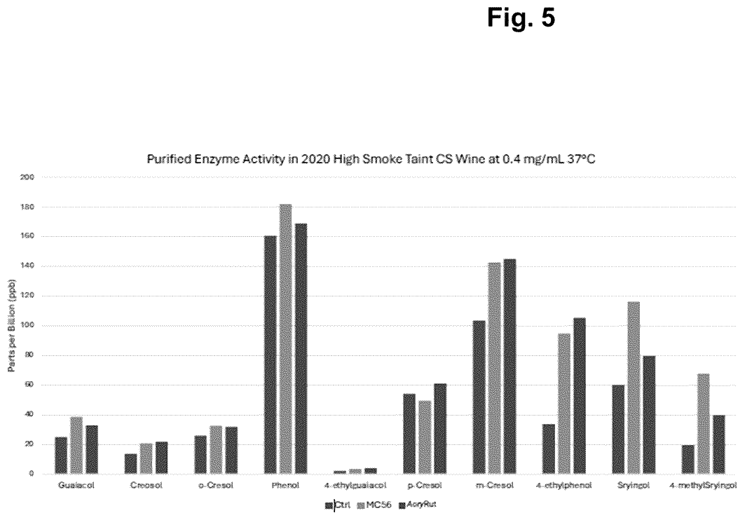

While stability and expression of AoryRut mutant MC56 were enhanced, the enzymatic activity of this mutant was maintained compared to wildtype (see FIG. 5).

Although the invention has been described with reference to the above examples, it will be understood that modifications and variations are encompassed within the spirit and scope of the invention. Accordingly, the invention is limited only by the following claims.

1-74. (canceled)

75. A method of enzymatically hydrolyzing volatile phenols from one or more phenolic glycosides in a fruit product, a fermented fruit product, a fruit fermentation apparatus, and/or a fruit fermentation container comprising:
incubating the volatile phenols in the fruit product, fermented fruit product, fruit fermentation apparatus, and/or fruit fermentation container with at least one glucosidase and at least one rutinosidase, wherein the enzymatic hydrolysis converts the one or more phenolic glycosides to volatile phenols and glycosides.

76. The method of claim 75, wherein the fruit product, fermented fruit product, fruit fermentation apparatus, and/or fruit fermentation container was smoke-exposed prior to the enzymatic hydrolysis.

77. The method of claim 75, wherein the at least one glucosidase has the amino acid sequence of SEQ ID NO. 1.

78. The method of claim 75, wherein the at least one rutinosidase has the amino acid sequence of SEQ ID NO. 78.

79. The method of claim 75, wherein the one or more phenolic glycosides are selected from glucosides, gentiobiosides, and rutinosides.

80. The method of claim 75, wherein the one or more phenolic glycosides are selected from the group consisting of guaiacol glucoside, guaiacol gentiobioside, guaiacol rutinoside, 4-methylguaiacol glucoside, 4-methylguaiacol gentiobioside, 4-methylguaiacol rutinoside, 4-ethylguaiacol glucoside, 4-ethylguaiacol gentiobioside, 4-ethylguaiacol rutinoside, cresol-p glucoside, cresol-p gentiobioside, cresol-p rutinoside, cresol-m glucoside, cresol-m gentiobioside, cresol-m rutinoside, cresol-o glucoside, cresol-o gentiobioside, cresol-o rutinoside, phenol glucoside, phenol gentiobioside, phenol rutinoside, 4-ethylphenol glucoside, 4-ethylphenol gentiobioside, 4-ethylphenol rutinoside, syringol glucoside, syringol gentiobioside, syringol rutinoside, 4-methyl syringol glucoside, 4-methyl syringol gentiobioside, and 4-methylsyringol rutinoside.

81. The method of claim 75, further comprising removing the smoke-associated volatile phenols and/or the phenolic glycoside from the fruit product and/or fermented fruit product, wherein the method comprises using one or more of filtration with activated carbon, reverse osmosis with activated carbon, yeast lees, cell walls, an enzyme, a cyclodextrin polymer and a molecularly imprinted polymer.

82. The method of claim 75, wherein the fruit product and/or fermented fruit product consists of or is derived from one or more fruits selected from the group consisting of a grape, an apple, a blueberry, a blackberry, a raspberry, a currant, a strawberry, a cherry, a pear, a peach, a nectarine, an orange, a pineapple, a mango, and a passionfruit.

83. The method of claim 75, wherein the fruit product or fermented fruit product is selected from the group consisting of a fruit homogenate, a fruit juice, a fruit pulp, a fruit skin, a fruit peel, a fruit seed, a fruit concentrate, and combinations thereof.

84. The method of claim 75, wherein the fermented fruit product is a fermented beverage selected from the group consisting of a table wine, dessert wine, fortified wine, sparkling wine, beer, spirits, cider, mead, liqueurs, sake, and brandy.

85. The method of claim 84, wherein the table wine is a red wine, a white wine, or a rosé wine.

86. The method of claim 85, wherein the red wine is selected from the group consisting of Cabernet Sauvignon, Alicante Henri Bouschet, Barbera, Bobal, Cabernet Franc, Carignan, Cinsaut, Malbec, Douce noir, Gamay, Grenache, Isabella, Merlot, Montepulciano, Mourvedre, Pinot noir, Sangiovese, Syrah, Tempranillo, Zinfandel, Aglianico, Blaufrankisch, Bordo, Carmenere, Castelão, Concord, Corvina Veronese, Criolla Grande, Croatina, Dolcetto, Dornfelder, Marufo, Mencia, Black Muscat, and Nebbiolo.

87. The method of claim 85, wherein the rose wine is selected from the group consisting of Provence Rosé Fresh, Grenache Rosé, Sangiovese Rosé, Syrah Rosé, Zinfandel Rosé, and Cabernet Sauvignon Rosé.

88. The method of claim 85, wherein the white wine is selected from the group consisting of Chardonnay, Sauvignon Blanc, Pinot Grigio, Moscato, Riesling, and Chenin Blanc.

89. The method of claim 75, wherein the fruit fermentation apparatus and/or the fruit fermentation container comprises a crusher, a destemmer, a fermentation vessel, a press, a pump, an airlock, a fermentation lock, a hydrometer, a refractometer, a thermometer, a primary fermenter, a secondary fermenter, a bottle, a barrel, a demijohn, a keg, a fermentation bucket, and/or a cork.

90. The method of claim 75, wherein 80% or more of the one or more phenolic glycosides are converted to volatile phenols and glycosides.

91. The method of claim 75, wherein the enzymatic hydrolysis converts 150% or more of the one or more phenolic glycosides to volatile phenolics and glycosides when compared to using acid hydrolysis on the same fruit product and/or fermented product thereof.

92. The method of claim 75, wherein the enzymatic hydrolysis has a mean relative hydrolysis efficiency that is about 1.35 when compared to using acid hydrolysis on the same fruit product and/or fermented product thereof.

93. A method of enzymatically hydrolyzing volatile phenols from one or more phenolic glycosides in a fruit product, a fermented fruit product, a fruit fermentation apparatus, and/or a fruit fermentation container comprising:
incubating the volatile phenols in the fruit product, fermented fruit product, fruit fermentation apparatus, and/or fruit fermentation container thereof with a composition comprising at least one glucosidase and at least one rutinosidase, wherein the composition comprises 0.001 mg/ml to 50 mg/ml of the at least one glucosidase and 0.001 mg/ml to 50 mg/ml of the at least one rutinosides.

94. A method of enzymatically hydrolyzing volatile phenols from one or more phenolic glycosides in a fruit product a fermented fruit product, a fruit fermentation apparatus, and/or a fruit fermentation container comprising:
incubating the volatile phenols in the fruit product, fermented fruit product, fruit fermentation apparatus, and/or fruit fermentation container with a composition comprising at least one glucosidase and at least one rutinosidase, wherein the composition comprises 0.01 mg/ml to 5 mg/ml of the at least one glucosidase and 0.01 mg/ml to 5 mg/ml of the at least one rutinosides.
